(19) United States
(12) Patent Application Publication
(10) Pub. No.: US 2005/0107738 A1
(43) Pub. Date: May 19, 2005

(54) Occludable intravascular catheter for drug delivery and method of using the same

(76) Inventors: Charles R. Slater, Fort Lauderdale, FL (US); Brett E. Naglreiter, Hollywood, FL (US); Scott L. Jahrmarkt, Miami Beach, FL (US); Thomas O. Bales, Coral Gables, FL (US); Banning G. Lary, Miami, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/922,221

(22) Filed: Aug. 19, 2004

Related U.S. Application Data
(63) Continuation-in-part of application No. 10/358,523, filed on Feb. 5, 2003.
Application No. 10/358,523 is a continuation-in-part of application No. 09/898,867, filed on Jul. 3, 2001.
(60) Provisional application No. 60/225,172, filed on Aug. 14, 2000.
(60) Provisional application No. 60/221,469, filed on Jul. 26, 2000.
(60) Provisional application No. 60/219,931, filed on Jul. 21, 2000.

(30) Foreign Application Priority Data
Feb. 24, 2004 (WO) PCTUS04/03249

(51) Int. Cl. A61M 29/00

(52) U.S. Cl. 604/96.01

(57) ABSTRACT

Methods and apparatus for treating the interior of a blood vessel include a variety of catheter designs, methods and apparatus for occluding a blood vessel, methods and apparatus for locating an occlusion device, methods and apparatus for locating a treating device at the site of blood vessel tributaries, and methods and apparatus for dispensing treating agent.

### RELATED U.S. APPLICATION DATA

This application 1) is a continuation-in-part of U.S. application Ser. No. 10/358,523 filed on Feb. 5, 2003, which is a continuation-in-part of U.S. application Ser. No. 09/898,867 filed on Jul. 3, 2001, which claims priority under 35 U.S.C. §119(e) to a) U.S. Provisional Application No. 60/225,172 filed on Aug. 14, 2000, b) U.S. Provisional Application No. 60/221,469 filed on Jul. 26, 2000, and c) U.S. Provisional Application No. 60/219,931 filed on Jul. 21, 2000, and 2) claims the benefit of priority under 35 U.S.C. 119(a)-(d) to PCT/US04/03249 filed Feb. 24, 2004, which are herein incorporated in their entirety by reference.

### FIELD OF THE INVENTION

The invention relates to the treatment and correction of venous insufficiency. More particularly the invention relates to a minimally invasive procedure using a catheter-based system to treat the interior of a blood vessel. The invention has particular application to varicose veins although it is not limited thereto.

### BACKGROUND OF THE INVENTION

The human venous system of the lower limbs consists essentially of the superficial venous system and the deep venous system with perforating veins connecting the two systems. The superficial system includes the long or great saphenous vein and the short saphenous vein. The deep venous system includes the anterior and posterior tibial veins which unite to form the popliteal vein, which in turn becomes the femoral vein when joined by the short saphenous vein.

The venous systems contain numerous one-way valves for directing blood flow back to the heart. Venous valves are usually bicuspid valves, with each cusp forming a sac or reservoir for blood which, under pressure, forces the free surfaces of the cusps together to prevent retrograde flow of the blood and allow antegrade flow to the heart. An incompetent valve is a valve which is unable to close because the cusps do not form a proper seal and retrograde flow of blood cannot be stopped.

Incompetence in the venous system can result from vein dilation. Separation of the cusps of the venous valve at the commissure may occur as a result. Two venous diseases which often involve vein dilation are varicose veins and chronic venous insufficiency.

The varicose vein condition includes dilatation and tortuosity of the superficial veins of the lower limb, resulting in unsightly discoloration, pain and ulceration. Varicose veins often involve incompetence of one or more venous valves, which allow reflux of blood from the deep venous system to the superficial venous system or reflux within the superficial system.

Varicose veins are compatible with long life and rarely cause fatal complications, but the condition significantly decreases the quality of life. Patients complain primarily of leg fatigue, dull, aching pains, ankle swelling and ulcerations. Occasionally, thrombosis occurs in dilated subcutaneous channels, resulting in local pain, induration, edema, inflammation, and disability. In addition to those problems, the high visibility of the unattractive rope-like swellings and reddish skin blotches causes considerable distress for both men and women. Lastly, varicose eczema, which is a local reddened swollen and itching skin condition can occur and can spread to distant parts of the body (called an “Id reaction”).

Phlebosclerosis, the destruction of venous channels by the injection of sclerosing agents, has been used to treat varicose veins since 1853, when Cassaignae and Ebout used ferric chloride. Sodium salicylate, quinine, urea, and sodium chloride have also been used, but the agent more recently favored is sodium tetradecyl sulfate. In order for phlebosclerosis to be effective, it is necessary to evenly dispense the sclerosing agent throughout the wall of the vein without using toxic levels of the sclerosing agent. This is not particularly difficult for the smaller veins. However, it is quite difficult or nearly impossible in larger veins. When a larger vein is injected with a sclerosing agent, the sclerosing agent is quickly diluted by the substantially larger volume of blood which is not present in smaller veins. The result is that the vein is sclerosed (injured) only in the vicinity of the injection. If the procedure is continued, and the injections are far apart, the vein often assumes a configuration resembling sausage links. The problem cannot be cured by injecting a more potent solution of sclerosing agent, because the sclerosing agent may become toxic at such a concentration.

U.S. Pat. No. 5,676,962 discloses an injectable micro foam containing a sclerosing agent. The microfoam is injected into a vein where it expands and, theoretically, achieves the same results as a larger quantity of sclerosing agent without the toxicity. Such foam is presently manufactured under the trademark Varisolve® by Provensis, Ltd., London, England. Recent clinical trials of the foam indicate a success rate of 81%.

Until recently, the preferred procedure for treating the great saphenous vein was surgical stripping. This highly invasive procedure involves making a 2.5 cm incision in the groin to expose the saphenofemoral junction, where the great saphenous vein and its branches are doubly ligated en masse with a heavy ligature. The distal portion of the vein is exposed through a 1-cm incision anterior to the medial malleolus, and a flat metal or plastic stripper is introduced to exit in the proximal saphenous vein. The leg is held vertically for 30 seconds to empty the venous tree before stripping the vein from the ankle to the groin. If the small saphenous vein is also incompetent, it is stripped at the same time from an incision posterior to the lateral malleolus to the popliteal space. After stripping the veins, the leg is held in the vertical position for three to four minutes to permit broken vessel ends to retract, constrict, and clot.

After the stripping procedure, collateral veins are removed by the avulsion-extraction technique. By working through small (5 to 8 mm) transverse incisions, segments of vein 10 to 20 cm long can be removed by dissecting subcutaneously along the vein with a hemostat, and then grasping, avulsing, and removing the vein. With practice, long segments of vein in all quadrants can be removed through these small incisions. No attempt is made to ligate the branches or ends of the veins, since stripping has shown it to be unnecessary. Bleeding is controlled by elevation and pressure for two to four minutes. As many as 40 incisions are made in severe cases, but their small size and transverse direction permit closure with a single suture.

Before closure of the incisions, a rolled towel is rolled repeatedly from the knee to the ankle and from the knee to the groin to express any clots that may have accumulated. The groin incision is approximated with three 5-0 nylon mattress sutures and all other incisions are closed with a single suture.

As can be readily appreciated, the stripping and avulsion-extraction procedures are relatively invasive and require significant anesthesia. It can therefore be appreciated that it would be desirable to provide an alternative, less invasive procedure which would accomplish the same results as stripping and avulsion-extraction.

Recently, a number of patents have issued disclosing the treatment of varicose veins with RF energy. Illustrative of these recent patents are: U.S. Pat. No. 6,200,312 entitled “Expandable Vein Ligator Catheter Having Multiple Electrode Leads”; U.S. Pat. No. 6,179,832 entitled “Expandable Catheter Having Two Sets of Electrodes”; U.S. Pat. No. 6,165,172 entitled “Expandable Vein Ligator Catheter and Method of Use”; U.S. Pat. No. 6,152,899 entitled “Expandable Catheter Having Improved Electrode Design, and Method for Applying Energy”; U.S. Pat. No. 6,071,277 entitled “Method and Apparatus for Reducing the Size of a Hollow Anatomical Structure”; U.S. Pat. No. 6,036,687 entitled “Method and Apparatus for Treating Venous Insufficiency”; U.S. Pat. No. 6,033,398 entitled “Method and Apparatus for Treating Venous Insufficiency Using Directionally Applied Energy”; U.S. Pat. No. 6,014,589 entitled “Catheter Having Expandable Electrodes and Adjustable Stent”; U.S. Pat. No. 5,810,847 entitled “Method and Apparatus for Minimally Invasive Treatment of Chronic Venous Insufficiency”; U.S. Pat. No. 5,730,136 entitled “Venous Pump Efficiency Test System And Method”; and U.S. Pat. No. 5,609,598 entitled “Method and Apparatus for Minimally Invasive Treatment of Chronic Venous Insufficiency”. These patents generally disclose a catheter having an electrode tip which is switchably coupled to a source of RF energy. The catheter is positioned within the vein to be treated, and the electrodes on the catheter are moved toward one side of the vein. RF energy is applied to cause localized heating and corresponding shrinkage of the adjacent venous tissue. After treating one section of the vein, the catheter can be repositioned to place the electrodes to treat different sections of the vein.

Although this procedure has gained acceptance and is less invasive than the stripping and avulsion-extraction procedures, there are several disadvantages to it. In particular, RF treatment is actually quite slow and painful and the patient must be sufficiently anaesthetized along the entire length of the veins to be treated. In addition, repositioning the catheter is time consuming thus requiring anesthesia for a prolonged period. Moreover, the RF treatment is incomplete, as only a portion of the vein wall is actually treated, i.e. the portion contacting the electrode. The partially treated vein may eventually recanalize. Furthermore, tributary veins remain unaffected and must be treated separately. In addition, for even and consistent cauterization, RF treatment requires that the practitioner be keenly aware of the procedure time. If RF energy is applied for too long, it can cause undesired burns. If RF energy is not applied long enough, the treatment is ineffective.

In addition to RF treatment, laser treatment has been used with some success. Laser treatment shares many of the disadvantages of RF treatment. In particular, as with the RF devices, the practitioner must be very careful as to the intensity and duration of the treatment to assure that the treatment is effective but without causing undesired burns.

Parent application Ser. No. 09/898,867 discloses an apparatus for delivering an intravascular drug such as a sclerosing agent (or a microfoam sclerosing agent) to a varicose vein. The apparatus includes a catheter having three concentric tubes. The innermost tube has a guide wire lumen and an inflation lumen. The distal end of the innermost tube has an integral inflatable occlusion balloon in fluid communication with the inflation lumen. The intermediate tube has a lumen through which the innermost tube extends. The distal end of the intermediate tube has a self-expanding balloon with a plurality of fluid pores in fluid communication with the intermediate tube lumen. The outer tube has a lumen through which the intermediate tube extends. Sclerosing agent is dispensed through the intermediate tube to pores located at the distal end of the intermediate tube or in the self-expanding balloon. Veins are sclerosed as the self-expanding balloon is pulled through and ultimately out of the vein.

While particular methods and apparatus were disclosed in the parent application for occluding the blood vessel, dispensing sclerosing agent, and locating tributaries, it will be appreciated that it would be desirable to have additional manners of accomplishing the same.

### SUMMARY OF THE INVENTION

In accordance with the present invention, an apparatus is provided which includes a catheter device having three concentric tubes: an inner tube, an outer tube, and an intermediate tube. Each tube has a proximal end and a distal end with a lumen extending therethrough. As used herein, the term proximal means closest to the practitioner and the term distal means farthest from the practitioner when the apparatus is in use. An inflatable balloon is located at or near the distal end of inner tube and a fluid valve is coupled to the proximal end of the inner tube. The balloon is inflated by injecting fluid through the valve and is held in an inflated condition by closing the valve. A fluid outlet is located at or near the distal end of the intermediate tube and a “plunger” or piston is coupled to the proximal end of the intermediate tube. The plunger is movable within the outer tube defining a fluid reservoir of varying size between the proximal end of the outer tube and the plunger. The plunger permits fluid communication between the fluid reservoir and the lumen of the intermediate tube. The proximal end of the outer tube is provided with a trifurcated fitting including a Touhy-Borst type connector. The proximal end of the inner tube extends through the Touhy-Borst connector which provides a fluid seal between the inner tube and the outer tube and which locks the inner tube in position relative to the outer tube. A pullwire is coupled to the plunger and extends through a central port of the trifurcated fitting which maintains a fluid seal between the pullwire and the outer tube. The third port of the trifurcated fitting is provided with a female Luer with a check valve which permits one-way fluid access into the fluid reservoir. According to one embodiment, the distal end of the inner tube is provided with a radiopaque tip and a safety wire extends within the inner tube providing the inner tube with stiffness and maneuverability for precise placement of the inflatable balloon. The wire is bonded to or captures the entire device, thereby helping to keep it together. The outer tube may be transparent and provided with a plurality of movable exterior markers which are useful in performing the methods of the invention.

According to alternate embodiments of the apparatus, other types of tracking devices may be used at the tip of the inner tube rather than the radiopaque tip. Examples of such devices include an LED or an illuminated fiber optic which is visible through the skin, or a magnet which can be detected with an electromagnetic sensor.

Methods of the invention include examining the patient and marking the patient's leg to indicate the entry site, the occlusion site and important sites (e.g. tributaries) along the blood vessel. The distal end of the outer tube is placed adjacent to the entry site and the inner tube and intermediate tube are extended outside the patient along the leg to the occlusion site. The intermediate tube is then drawn back from the occlusion site to the first important site marking proximal of the occlusion site. One of the movable exterior markers on the outer tube is then moved to the position occupied by the plunger. The intermediate tube is then moved to the next proximal important site marking on the leg and another marker on the outer tube is moved to the corresponding position of the plunger. These steps are repeated until all of the important site markings have been recorded with the movable markers on the outer tube. The catheter is then reset so that the distal ends of the inner tube and intermediate tube are adjacent to each other. A 10 cc to 20 cc syringe is loaded with sclerosing agent and is attached to the female Luer. While holding the catheter in an upward direction, 10 cc of sclerosing agent is injected into the fluid reservoir and the intermediate tube until a few drops exit the fluid outlet of the intermediate tube and the tubes are purged of air bubbles. If necessary, the syringe is reloaded with additional sclerosing agent.

The inner and intermediate tubes are then inserted through a hemostasis valve or cut-down into the blood vessel and maneuvered through the vessel until the distal end of the outer tube abuts the vessel or hemostasis valve. The balloon is then inflated using a 3 cc to 5 cc syringe coupled to the proximal end of the inner tube. Infusion of sclerosing agent is commenced by pulling the pullwire so that the plunger is moved proximally forcing fluid out of the fluid reservoir through the intermediate tube and out of the fluid outlets at the distal end of the intermediate tube. When the plunger reaches one of the markers on the outer tube, additional sclerosing agent may be injected using the 10 cc to 20 cc syringe. The plunger is then moved to the next marker and additional sclerosing agent is injected. After all of the markers have been passed by the plunger, the balloon is deflated and the catheter device is removed from the patient.

The occlusion devices of the present invention include: sponges, umbrellas, cages, chemical sealants, ligation, and a suction device. The umbrella or cage designs may incorporate elastic or superelastic struts, a tubular inflatable cuff, or a wire hoop with a basket.

The methods for locating the occlusion device according to the invention include: ultrasound, palpation, fluoroscopic and magnetic resonance imaging, placing a bright light (e.g. LED) at the end of the occlusion device, pressure monitoring, and a technique similar to the placement of a “wedge catheter”.

The methods for locating tributaries include two types: one involves pre-marking on the patient's skin, and the other does not use marking. The pre-marking methods include locating the tributaries via ultrasound, transillumination, or other type of imaging, and marking the patient's skin at the locations of the tributaries. After pre-marking several additional methods can be used. One method involves marking the treating device by placing the treating device on the patient's skin and marking it in locations that align with the marks on the patient's skin. A second method following pre-marking involves using a bright light at the tip of the drug delivery device. A third method following pre-marking involves using ultrasound to locate the tip of the drug delivery device. A fourth method following pre-marking involves using palpation to locate the tip of the drug delivery device. A fifth method following pre-marking involves using a magnet at the tip of the drug delivery device and a magnetic follower on the patient's skin. Several different types of magnetic followers are provided.

The methods for locating tributaries without pre-marking include: ultrasound imaging during the procedure, placing a light source at the tip of the drug delivery device bright enough to illuminate the tributaries through the patient's skin, external illumination with or without an image intensifying system, real time fluoroscopy or other type of imaging, and pressure gradient detection.

Further embodiments of catheter-based treating devices include: a catheter having an atraumatic floppy guide wire tip attached to the distal end of an inflatable occlusion balloon, a dual monorail catheter system, a two-way single monorail catheter system, a two-way clip-on catheter system, and a multi-perforated catheter which does not move during drug delivery.

In one embodiment of the invention a device for treating blood vessels is provided. The device comprises an elongate body having a proximal end, a distal end, and an infusion lumen extending therethrough, a plurality of elution holes in valved communication with the infusion lumen and a wall which is movable between a first position in which the wall blocks communication between the infusion lumen and the elution holes and a second position in which the infusion lumen is in communication with the elution holes. The wall may be movable in response to a change in pressure. The wall may also be movable in response to introduction of an inflation media. In one embodiment of the device, the wall is in the form of an inflatable tube. The device may further comprise a side lumen on the body and where the inflatable tube is positioned within the side lumen. The tube may also be positioned within an infusion lumen. The inflatable tube has an axial length of at least 0.5 cm. In one embodiment, the total fluid resistance of the elution holes is about equal to or greater than the total fluid resistance of the infusion lumen. In some embodiments, the total fluid resistance of the elution holes is at least about 125% of the fluid resistance of the infusion lumen. In some embodiments, the average hydraulic diameter of the elution holes is less than about 0.010 inches. In other embodiments, the average hydraulic diameter is less than about 0.004 inches. The average spacing between the elution holes is within the range of from about 1 cm to about 2 cm. The device may further comprise an inflatable occlusion balloon carried by the distal end of the body. The device may also comprise a guide wire lumen extending axially through at least a portion of the elongate body. The inflatable tube in some embodiments has a deflated diameter, side lumen has an inside diameter, and the deflated diameter is no more than about 75% of the inside diameter.

In another embodiment of the invention, a fluid delivery catheter is provided. The fluid delivery catheter comprises an elongate flexible tubular body, having a proximal end and a distal end, an infusion lumen extending through the body from the proximal end in the direction of the distal end, at least two infusion ports on the tubular body and an inflatable tube within the tubular body, wherein at least one infusion port is in communication with the infusion lumen when the inflatable tube is in a first inflation state. Then the infusion port is isolated from the infusion lumen when the inflatable tube is in a second inflation state. The catheter may further comprise a vascular occlusion balloon at the distal end of the tubular body. The catheter may also comprise a proximal manifold having an infusion port in communication with the infusion lumen and an inflation port in communication with the occlusion balloon.

In another embodiment of the invention, a method of treating a body lumen is provided. The method comprises providing a catheter with an infusion lumen and a plurality of elution holes in selective communication with the infusion lumen, the catheter having a first configuration adapted to resist flow through at least one elution hole and a second configuration adapted to allow flow through at least one elution hole, inserting the catheter into a patient, introducing a therapeutic fluid into the infusion lumen and changing the catheter from the first configuration to the second configuration to permit escape of therapeutic fluid through the at least one elution hole. The step of changing the catheter may comprise moving a movable wall from a first position in which communication between the at least one elution hole and the infusion lumen is interrupted, to a second position in which the at least one elution hole is in communication with the infusion lumen. Furthermore, the step of changing the catheter may also comprise deflating a tubular flow regulator.

In another embodiment, a method of introducing a therapeutic agent into a vein is provided, The method comprises introducing a catheter into the vein, the catheter having a plurality of infusion ports and an infusion lumen, activating an inclusion device on the catheter to include blood flow within the vein, removing a barrier from at least one of the plurality of infusion ports and infusing a therapeutic agent from the infusion lumen through the ports and into the vein. The introducing step may comprise introducing the catheter into the saphenous vein. Introducing the catheter into the saphenous vein may occur in the vicinity of the knee or the vicinity of the ankle. The activation of the inclusion device may comprise inflating an inclusion balloon and/or isolating the saphenofemoral junction from the infusion ports. The step of removing a barrier may comprise deflating an elongate tubular bladder. The method may further comprise enhancing drainage of the vein by raising the position of the vein relative to the location of the occlusion device. The method may also comprise lowering the position of the vein relative to the location of the occlusion device to facilitate migration of the therapeutic agent along the vein wherein the therapeutic agent is a foam. The method may also comprise maintaining a raised position of the vein relative to the location of the occlusion device to facilitate migration of the therapeutic agent to the saphenofemoral junction.

In another embodiment, a method of inhibiting retrograde flow of body fluid through effluent ports and into the infusion lumen of a catheter is provided. The method comprises the steps of providing a fluid delivery catheter, having an elegant body, at least one effluent port on the body and an infusion lumen extending within the body, inflating a flow regulator within the tubular body to isolate the effluent port from the infusion lumen and introducing the catheter into a patient in a location that exposes the catheter to a body fluid wherein the flow regulator inhibits retrograde flow of body fluid through the effluent port and into the infusion lumen. The step of inflating a flow regulator may comprise inflating an elongate tubular balloon. The method may additionally comprise the step of deflating the flow regulator to place the effluent port in communication with the infusion lumen.

Additional features and advantages of the invention will become apparent to those skilled in the art upon reference to the detailed description taken in conjunction with the provided figures.

### BRIEF DESCRIPTION OF THE DRAWINGS

FIG. 1 is a schematic side elevational view of a first catheter device according to the invention with the inner and intermediate tubes withdrawn.

FIG. 2 is a schematic side elevational view of the first catheter device according to the invention with the inner and intermediate tubes extended.

FIG. 3 is a schematic side elevational view of the first catheter device according to the invention in use.

FIGS. 4A to 4E are schematic illustrations of the distal ends of the inner tube and intermediate tube of the first catheter device during use.

FIG. 5 is a schematic view of a sponge occlusion device in a state of partial deployment.

FIG. 6 is a schematic view of a first umbrella occlusion device in a state of partial deployment.

FIG. 7 is a schematic view of a second umbrella occlusion device in a state of partial deployment.

FIG. 8 is a schematic view of a third umbrella occlusion device in a state of partial deployment.

FIG. 9 is a schematic view illustrating deployment of a tissue sealant at an occlusion site.

FIG. 10 is a schematic view of a compression device at an occlusion site.

FIG. 11 is a schematic view illustrating venous ligation as an occlusion method.

FIG. 12 is a schematic view of a suction apparatus for occluding a blood vessel.

FIG. 13 is a schematic view illustrating the positioning of an occlusion device with the use of ultrasound.

FIG. 14 is a schematic view illustrating the positioning of an occlusion device with the use of palpation.

FIG. 15 is a schematic view illustrating the positioning of an occlusion device with the use of fluoroscopic imaging.

FIG. 16 is a schematic view illustrating the positioning of an occlusion device with the use of a bright light coupled to the occlusion device.

FIG. 17 is a schematic view illustrating the positioning of an occlusion device with the use of a pressure monitor.

FIG. 18 is a schematic view illustrating the positioning of an occlusion device with the use of a wedge placement technique.

FIG. 19 is a schematic view illustrating the pre-marking of a patient's leg indicating the locations of tributaries.

FIG. 20 is a schematic view illustrating the marking of a treatment device using the pre-marked leg as a guide.

FIG. 21 is a schematic view illustrating the location of a treatment device at a tributary using a first embodiment of a magnetic follower and the pre-markings on the patient's leg.

FIG. 22 is a schematic view illustrating the location of a treatment device at a tributary using a second embodiment of a magnetic follower and the pre-markings on the patient's leg.

FIG. 23 is a schematic view illustrating the location of a treatment device at a tributary using a third embodiment of a magnetic follower and the pre-markings on the patient's leg.

FIG. 24 is a schematic view illustrating the location of a treatment device at a tributary using a fourth embodiment of a magnetic follower and the pre-markings on the patient's leg.

FIG. 25 is a schematic view illustrating the location of a treatment device at a tributary using external IR illumination.

FIG. 26 is an enlarged, fragmentary schematic illustration of the distal end of a catheter having an atraumatic floppy guide wire tip attached to the distal end of an inflatable occlusion balloon.

FIG. 27 is a schematic illustration the distal end of a dual monorail catheter system.

FIG. 28 is a schematic illustration of the distal end of a single monorail catheter system.

FIG. 28A is a section taken along line A-A in FIG. 28.

FIG. 29 is a schematic illustration of the distal end of a clip-on catheter system.

FIG. 29A is a section taken along line A-A in FIG. 29.

FIG. 30 is a schematic illustration of another embodiment of the invention which utilizes a multi-perforated catheter which does not change position during drug delivery.

FIG. 31 is a schematic illustration of a multi-perforated weeping catheter.

FIG. 32 is a schematic illustration of a second embodiment of a multi-perforated weeping catheter.

FIG. 33 is a schematic perspective view of a portion of a third embodiment of a multi-perforated weeping catheter.

FIG. 34 is a longitudinal cross sectional view of a fourth embodiment of a multi-perforated weeping catheter.

FIG. 35 is a perspective view of the distal end of a fifth embodiment of a multi-perforated weeping catheter.

FIG. 36 is a side elevational view of the distal end of the fifth embodiment of a multi-perforated weeping catheter with its occlusion balloon inflated.

FIG. 37 is a section taken along line 37-37 in FIG. 36.

FIG. 38A is a side elevational schematic view of one embodiment of the invention with multiple elution holes along the length of the catheter.

FIG. 38B is a transverse cross sectional view taken along the line 38B-38B of FIG. 38A.

FIG. 38C is a fragmentary longitudinal cross sectinal view taken along the line 38C-38C of FIG. 38B.

FIG. 38C is a fragmentary longitudinal cross sectional view taken along the line 38C-38C of FIG. 38B.

FIG. 39 is a schematic view showing one embodiment of non uniform elution hole spacing in a catheter.

FIG. 40 is a schematic view showing one embodiment of non uniform elution hole size in a catheter.

FIGS. 41A and 41B are side elevational fragmentary schematic views of two embodiments of a porous elution region on an infusion catheter.

FIG. 41C is a cross sectional view taken along the line 41C-41C of FIG. 41A.

FIG. 41D is a cross sectional view taken along the line 41D-41D of FIG. 41B.

FIG. 42A is a side elevational schematic cross sectional view of one embodiment of a catheter showing a movable occluder in the first position.

FIG. 42B is a side elevational cross section as in FIG. 42A, showing the movable occluder in a second position.

FIGS. 43A to 43C depict another embodiment of a catheter comprising a movable occluder in closed, partially open and open positions, respectively.

FIGS. 44A to 44D depict sequential steps in the operation of another embodiment of a catheter comprising a movable occluder.

FIG. 45 illustrates one embodiment of a catheter comprising stops in the side lumen.

FIG. 46 shows one embodiment of the invention where occlusion surfaces are centrally aligned.

FIG. 47 show one embodiment of the invention where occlusion surfaces are eccentrically aligned.

FIG. 48 is a cross sectional schematic view of one embodiment of an occluder with a polygonal cross sectional shape.

FIG. 49 is a side elevational schematic fragmentary view of the proximal manifold having an occluder position indicator.

FIGS. 50A and 50B are schematic views as in FIG. 49, of various combined occluder actuator/indicators.

FIGS. 51A to 51C are longitudinal cross sectional schematic views of one embodiment of an alternative movable occluder.

FIGS. 52A and 52B are cross sectional schematic views of one embodiment of a distally anchored elastomeric occluder.

FIGS. 53A and 53B illustrate another embodiment of a distally anchored elastomeric occluder.

FIGS. 54A and 54B are longitudinal cross sectional views of one embodiment of the invention comprising an inflatable occlusion tube in a deflated and inflated state, respectively; FIGS. 54C and 54D are transverse cross sectional views of the catheters of FIGS. 54A and 54B, respectively.

FIGS. 55A and 55B are schematic transverse cross sectional views of one embodiment of the invention with a coaxially positioned occlusion tube.

FIGS. 56A and 56B are schematic axial cross sectional views of one embodiment of the invention with a concentric, eccentrically positioned occlusion tube.

FIGS. 57A and 57B are schematic views of one embodiment of the invention comprising a catheter with slit elution holes.

FIGS. 58A and 58B are schematic views showing various embodiments of slit elutions holes.

FIGS. 59A to 59D illustrate one embodiment of the invention comprising H-shaped slits on the catheter. FIGS. 59C and 59D are cross-sectional views of the catheter depicted in FIGS. 59A and 59B in a closed and open configuration, respectively.

FIGS. 60A to 60C are schematic views of another embodiment, comprising a catheter with a slotted overtube.

FIGS. 61A to 61E are schematic views of another embodiment, comprising a catheter with segmented elastic coverings.

FIG. 62A and 62B are schematic views of another embodiment of the invention, comprising a gate-type valve-controlled elution hole.

FIG. 63 is a schematic cross sectional view of one embodiment of the invention comprising a single filter within a side lumen of a catheter.

FIG. 64 is a schematic cross sectional view of one embodiment of the invention comprising multiple discrete filters within a side lumen of a catheter.

FIG. 65 is a side elevational view of one embodiment of the invention, comprising a catheter sheath introducer and a catheter with markers for indicating catheter position.

FIGS. 66A to 66C depict another embodiment of the invention comprising a catheter with a rotatable flow control; FIGS. 66B and 66C are transverse cross sectional views of the catheter from FIG. 66A in a closed and open configuration, respectively.

FIGS. 67A and 67B are schematic illustrations of one embodiment of the invention comprising a catheter with an inflatable balloon tip and a bladder tube occluder.

FIGS. 68A and 68B are frontal elevational and longitudinal cross sectional views of the catheter in FIGS. 67A and 67B.

FIGS. 69A and 68B are schematic longitudinal and axial cross sectional view depicting the configuration of the side lumen and elution holes.

FIG. 70 is a cross sectional view of the catheter along the distal catheter body and balloon assembly.

FIGS. 71A to 71D are cross sectional views of the balloon assembly.

FIG. 72 depicts an elevational view of one embodiment of the invention with access conduits in the trifurcated fitting of the catheter.

### DETAILED DESCRIPTION OF THE PREFERRED EMBODIMENT

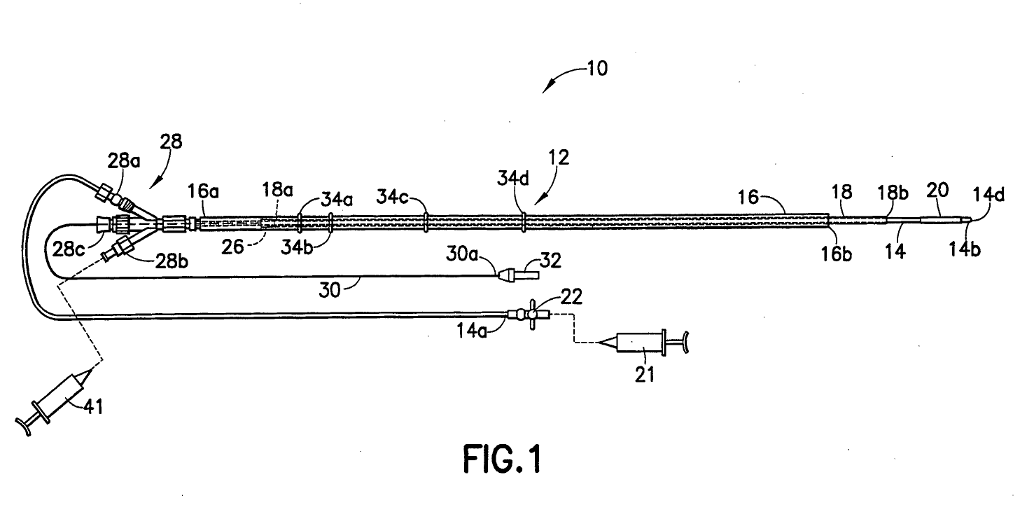

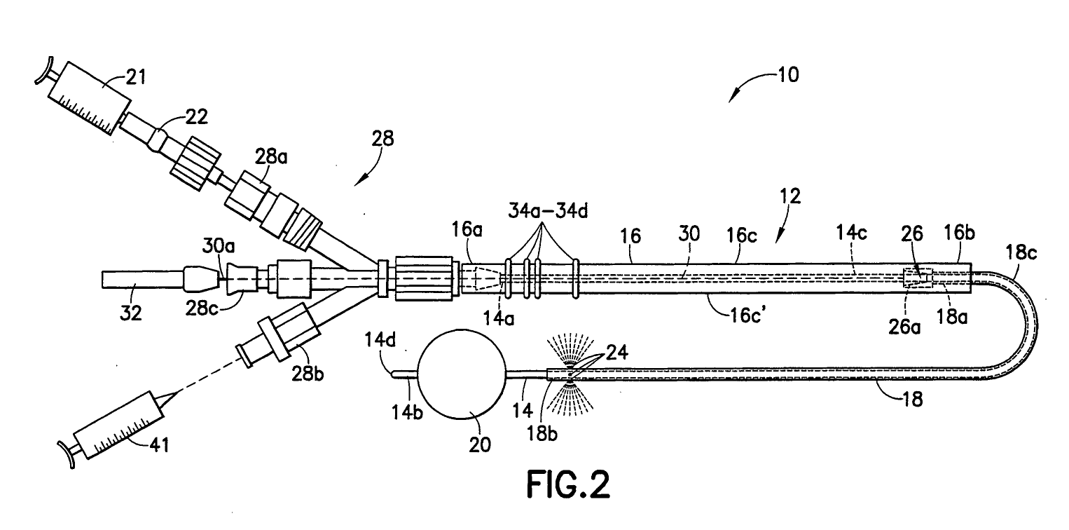

Referring now to FIGS. 1 and 2, one embodiment of the invention is an apparatus 10 comprising a catheter device 12 having three concentric tubes: an inner tube 14, an outer tube 16, and an intermediate tube 18. Each tube 14, 16, 18 has a proximal end 14a, 16a, 18a and a distal end 14b, 16b, 18b with a lumen 14c, 16c, 18c extending therethrough. As used herein, the term proximal means closest to the practitioner and the term distal means farthest from the practitioner when the apparatus is in use.

An inflatable balloon 20 is located at or near the distal end 14b of inner tube 14 and a fluid valve 22 is coupled to the proximal end 14a of the inner tube 14. The balloon 20 is inflated by injecting fluid (e.g. saline) through the valve 22 and is held in an inflated condition by closing the valve 22.

As seen best in FIG. 2, one or more fluid outlet(s) 24 are located at or near the distal end 18b of the intermediate tube 18 and a “plunger” 26 or piston is coupled to the proximal end 18a of the intermediate tube 18. According to the presently preferred embodiment, the fluid outlets 24 include a plurality of radial outlets and a fluid seal (not shown) that closes the annular space between the tube 14 and the tube 18 at a location distal of the outlets 24. The fluid seal (not shown) is heat formed and makes a sliding (dynamic) seal. The plunger 26 is movable within the outer tube 16 defining a fluid reservoir 16c′ of varying size between the proximal end 16a of the outer tube 16 and the plunger 26. For example, FIGS. 1 and 2 illustrate two extreme locations of the plunger 26, FIG. 1 showing a small reservoir and FIG. 2 showing a large reservoir. The plunger 26 permits fluid communication between the fluid reservoir 16c′ and the lumen 18c of the intermediate tube 18. According to the one preferred embodiment, the plunger 26 is provided with an indication 26a as seen best in FIG. 2. According to the presently preferred embodiment, the indication 26a is a sealing O-ring contrasting in color to that of the plunger 26.

The proximal end 16a of the outer tube 16 is provided with a trifurcated fitting 28 including a Touhy-Borst type connector 28a, a female Luer 28b with check valve (not shown) and a Luer 28c housing a seal connector (not shown).

The proximal end 14a of the inner tube 14 extends through the Touhy-Borst connector 28a which provides a fluid seal between the inner tube 14 and the outer tube 16 and which selectively locks the inner tube 14 in position relative to the outer tube 16.

The female Luer 28b with check valve permits one-way fluid access into the fluid reservoir 16c′ of the outer tube 16.

A pullwire 30 is coupled to the plunger 26 and extends through the Luer 28c of the trifurcated fitting 28 which maintains a fluid seal between the pullwire 30 and the outer tube 16. The proximal end 30a of the pullwire 30 is provided with a handle 32. According to the presently preferred embodiment, the handle is a striking color (e.g. orange) so that it can be quickly located.

According to the presently preferred embodiment, the distal end 14b of the inner tube 14 is provided with a radiopaque tip 14d and a safety wire (not shown in FIGS. 1 or 2) extends within the inner tube 14 providing the inner tube with stiffness and maneuverability for precise placement of the inflatable balloon.

Further according to the presently preferred embodiment, the outer tube 16 is transparent and provided with a plurality of movable exterior markers 34a to 34d which are used in conjunction with the indication 26a on the plunger 26 in performing the methods of the invention described in more detail below. The presently preferred markers are elastic O-rings.

According to alternate embodiments of the apparatus, other types of tracking devices may be used at the distal end of the inner tube rather than the radiopaque tip. Examples of such devices include an LED or an illuminated fiber optic which is visible through the skin, or a magnet which can be detected with an electromagnetic sensor.

In one embodiment, the apparatus 10 comprises a first syringe 21 having a volume of about 1 mL to about 10 mL for inflating the balloon, and a second syringe 41 having a volume of about 5 mL to about 25 mL for injecting sclerosing agent. In one embodiment, the apparatus comprises a first syringe 21 having a volume of about 2 mL to about 5 mL and a second syringe 41 having a volume of about 10 mL to about 50 mL. In one embodiment, the apparatus 10 is intended for use with and thus also preferably includes two syringes, an about 3 cc to about 5 cc syringe 21 for inflating the balloon and an about 10 cc to about 20 cc syringe 41 for injecting sclerosing agent.

In one embodiment of the invention, although it is not necessary to perform the procedure in an operating room, it is considered prudent for the initial examination to be performed in an out-patient suite in a hospital or in an operating room in the event that any unforeseen events occur that may require surgical intervention.

In one embodiment, the patient is first examined under ultrasound, palpation, fluoroscopy or other means for venous valve insufficiency and varicose veins. If the physician determines that the patient is a candidate for closure of the saphenous vein as a means of eliminating the varicosities, the patient will be admitted for the procedure.

In one preferred embodiment, a photograph of the patient's leg is taken both before and after the procedure so that the results of the procedure can be readily ascertained.

The patient is preferably sedated with a mild sedative and/or pain medication such as Percocet, or the like, one hour prior to the procedure. An intravenous line may be inserted in the patient's arm and vital signs monitored throughout the procedure.

In one embodiment, while the patient is standing, the saphenofemoral junction is located using Doppler or other ultrasonic techniques and the skin marked over this junction with a washable marker. Similarly, the saphenous vein and its major tributary junctions is traced using ultrasound and its path marked on the surface of the skin with a marker.

If varicosities are present above the knee only, then the length of the saphenous vein from the knee to the groin will be treated either through a cut down to the saphenous vein or by a percutaneous stick into the saphenous vein (or both) using a catheter sheath introducer (“CSI”). If the disease is prevalent below the knee, then a similar incision or percutaneous stick will be made in the saphenous vein at the level of the ankle and the vein sclerosed from the ankle to the knee. If the disease is prevalent in both the upper and lower leg, then an incision or percutaneous stick will be made in the saphenous vein at the level of the ankle and the vein sclerosed from the ankle to the groin and the entire vein sclerosed.

The patient lies down with his/her leg elevated 30 to 45 degrees to allow blood to drain from the leg. The patient's leg is scrubbed with a standard surgical preparation medium, such as betadine, and the site prepared for an aseptic procedure. Non-iodine sterilization solutions may be used for patients with iodine allergies. Lidocaine or other local anesthetic is injected into the area around the vein with a small needle. A local anesthetic with epinephrine may be used to provide hemostatic control at the entry or insertion site.

In one embodiment, the apparatus 10 is examined prior to use to determine that it is functioning properly. This should include sliding the plunger in and out through the outer tube and dilating the balloon with about 3 cc of sterile saline to check for leakage.

The following procedure assumes that the patient's skin has been previously marked with the entry site, the occlusion site and important sites (e.g. tributaries) along the vessel. It also assumes that the catheter device can be laid down on the patient's leg while maintaining sterility.

With the inner tube 14 and the intermediate tube 18 drawn into the outer tube 16 as shown in FIG. 1, the distal end 16b of the outer tube 16 is positioned externally at the entry site (just proximal to the hemostasis valve of the CSI). While the outer tube 16 is maintained in position, the inner tube 14 and the intermediate tube 18 are extended distally out of the outer tube 16, by grasping and pulling the intermediate tube 18 in a distal direction until the balloon 20 is over the mark on the skin representing the occlusion site.

The inner tube 14 is locked in position by tightening the Touhy-Borst valve 28a. Locking the Touhy-Borst valve assures that when the apparatus is inserted into the leg, the balloon will inflate at the desired occlusion site. It also assures that the balloon will not migrate backwards when the sclerosing agent is dispensed.

Starting with the distal end 18b of the intermediate tube 18 abutting the balloon 20, the pullwire 30 is pulled such that the intermediate tube 18 retracts proximally until the fluid outlet 24 is located at the next marking on the patient's leg (e.g. a tributary site). With the apparatus in this position, the closest marker (o-ring) 34d is moved over the tube 16 until it is aligned with the indicia 26a on the plunger 26. The pullwire 30 is pulled again and this step is repeated for each of the marks on the patient's leg, using the O-rings 34c, 34b, 34a to mark the corresponding location of the plunger 26. It will be appreciated that the number of markers shown in the figures is arbitrary and more or fewer markers may be provided.

After all of the desired markers 34a-34d have been placed along the tube 16, the intermediate tube 18 is pulled distally until its distal end 18b abuts the balloon 20 as shown in FIG. 2.

As mentioned above, two syringes are used to operate the apparatus, an about 3 cc to about 5 cc syringe 21 to expand the balloon and an about 10 cc to about 20 cc syringe 41 to dispense the sclerosing agent. The smaller syringe is filled with sterile saline and attached to the fluid valve 22 (a Luer with a stop cock). The larger syringe is filled with sclerosing agent and attached to the female Luer 28b. While holding the intermediate tube 18 in an upward direction, about 10 cc of the sclerosing agent is injected through the check valve 28b into the reservoir 16c′ of the tube 16, through the plunger 26, and up through the tube 18 such that a few drops of fluid emerge from the fluid outlets 24 on the distal end of the tube 18. The physician should ensure that the tubes 16, 18 are purged of air bubbles. If necessary, the larger syringe is reloaded with additional sclerosing agent before proceeding.

The inner tube 14 and the intermediate tube 18 are then inserted into a percutaneous stick 40 in the saphenous vein 42 as shown in FIG. 3. The tubes 14, 18 are maneuvered to the occlusion location 44 preferably with the aid of the tip indicator 14d of the tube 14. As mentioned above, the tip indicator 14d may be radiopaque and thus located with fluoroscopy or other radiographic methods. Alternatively, the tip 14d may be provided with an LED or an optical fiber which causes it to glow bright enough to be seen through the skin. Still alternatively, the tip 14d may be magnetic and thus located with electromagnetic equipment. Outer tube 16 may be temporarily secured to the patient's leg during the procedure by securing the tube 16 with a suture, medical tape or a Velcro® strap.

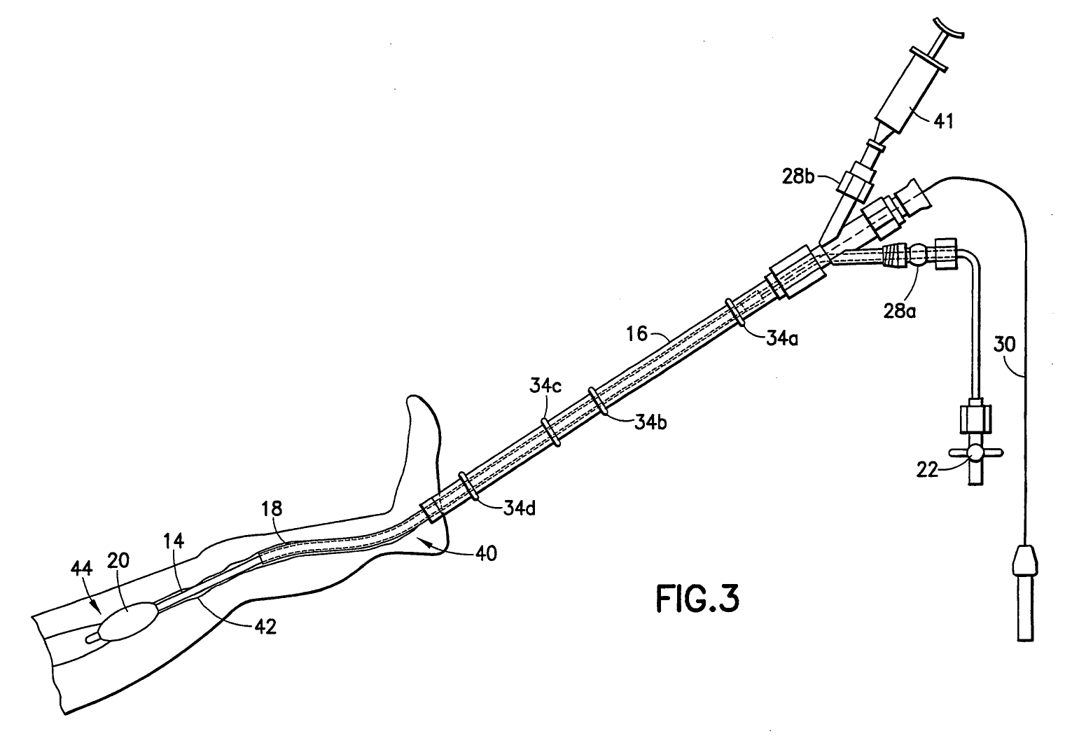

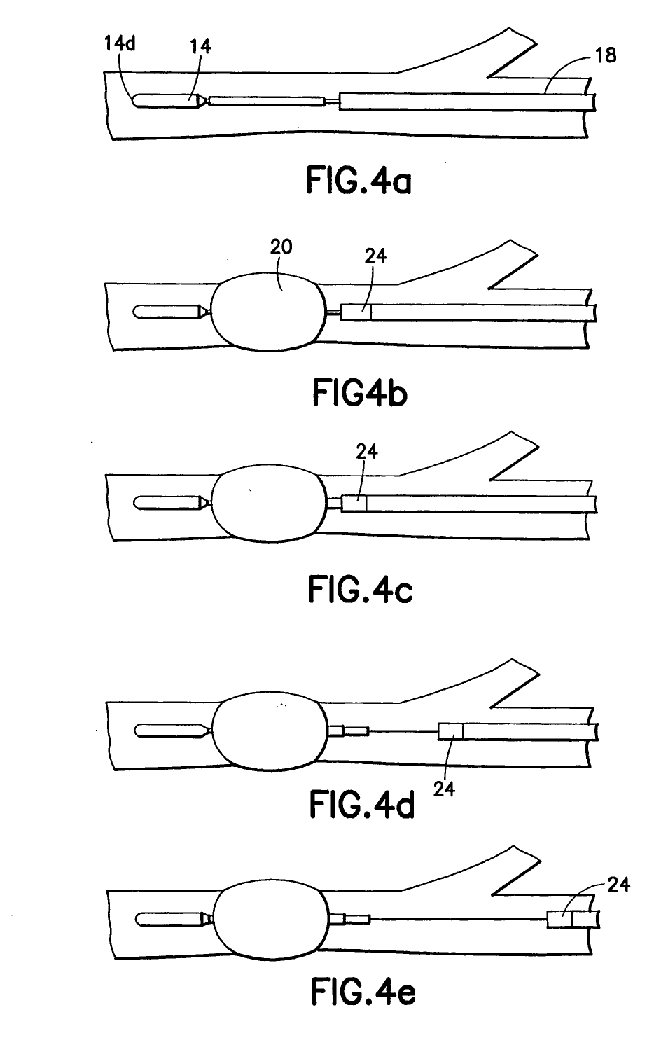

With the apparatus in position as shown in FIG. 4A, the balloon 20 is expanded with the small syringe as shown in FIG. 4B. According to the presently preferred embodiment, preferably no more than about 5 cc should be injected into the balloon which will expand to a diameter of approximately 21 mm upon injection of 5 cc. Table 1 illustrates a typical relationship between the injection volume and the balloon diameter.

TABLE 1

| Injection volume ±0.1 cc | Balloon Diameter ±1 mm |
|---|---|
| 1 | 12 |
| 2 | 15 |
| 3 | 18 |
| 4 | 19 |
| 5 | 21 |

The balloon is preferably inflated slowly with sterile saline or radiopaque media until it totally occludes the vessel. Ultrasound, fluoroscopy, palpation, tugging, etc. can be used to ensure that the balloon is adequately inflated. Once the balloon is inflated, the stopcock 22 is closed by rotating the stopcock 90°. Doppler ultrasound can also be used to check the absence of blood flow at the occlusion site.

The infusion procedure is begun by pulling the pullwire 30 back until the O-ring on the plunger 26 lines up with the first O-ring marker previously located on the tube 16. Pulling on the pullwire causes the plunger 26 to be moved toward the proximal end of the tube 16, which in turn forces the sclerosing agent out of the fluid outlets 24 in the distal end of the tube 18 which is also moved away from the balloon 20 as shown in FIG. 4C. This releases a controlled and evenly distributed amount of sclerosing agent which is well suited for sclerosing a vein with no tributaries. When the end of the tube 18 reaches a tributary, as shown in FIG. 4D and as indicated by the placement of the O-rings 34a-34d, it is desirable to release additional sclerosing agent to contract the tributary as well as the vein. This may be accomplished by injecting additional sclerosing agent with the large syringe which remains attached to the injection port 28b. After the additional sclerosing agent is released, movement of the tube 18 is resumed as shown in FIG. 4E.

Injection of this bolus of sclerosing agent may be directed and facilitated with a fork-like device (not shown) that compresses the outside of the leg on either side of the fluid outlets 24. A roller may also be used to force the sclerosing agent up the tributary. This process is repeated for other large tributaries. In one embodiment, a total of about 5 cc to about 100 cc of sclerosing agent is used during the procedure. In one embodiment, a total of about 10 cc to 75 cc of sclerosing agent is used. In one embodiment, preferably no more than about 20 cc of 0.5% sclerosing agent should be used in this procedure.

When the tube 18 is fully withdrawn, the balloon 20 is deflated by aspiration and the tube 14 is removed from the vein. The entry site may be sutured before dressing. However, according to the presently preferred embodiment, the size of the introducer is only 6-French which may produce a sufficiently small wound so as not to require suturing. However, the leg is preferably immediately wrapped in a gauze-type dressing (e.g., KERLIX® available from Kendall Co., Walpole, Mass.). A length of foam rubber padding is preferably placed over the gauze and over the saphenous vein that was sclerosed. An elastic bandage (e.g., COACH® or ACE®) is preferably placed over the foam rubber to keep it in place. An additional elastic bandage may be placed over the first elastic bandage to ensure that the vein remains compressed and that blood does not flow back into the treated veins.

The patient should be advised to rest with his/her leg elevated for approximately 30 minutes. The patient can then walk to the car, elevate the leg in the car and then keep the leg elevated in bed overnight. Occasional flexure of the foot, ankle and leg should be encouraged. It is preferred that the patient be re-examined the following day. The dressings should then be replaced and the patient instructed on how to self apply new dressings and bandages. The dressings, foam pads and bandages may be kept in place for five to seven days. After five to seven days, the patient should be re-examined and, if indicated, the dressings and foam removed. The compression bandage should be worn for an additional week.

The patient should be asked to return for follow-up at one month and three months if indicated. The patient may also be asked to return at one year to evaluate the long-term effectiveness of the procedure.

The benefits of the methods and apparatus of the invention include:

Sclerosing agents are painless in the vascular system as compared to laser or RF ablation that can be extremely painful.

The occlusion balloon prevents the sclerosing agent from entering the deep venous system via the saphenofemoral or saphenopopliteal junctions.

The catheter is 6-Fr in diameter and is easily maneuvered through the vein.

Only one injection of anesthesia is required at the puncture site, resulting in less pain and toxicity to the patient.

Venous access via a small cut down or by use of a catheter sheath introducer produces a very minimal scar, resulting in a better cosmetic impact.

The recovery time is faster with fewer cosmetic complications as compared to stripping.

Tributaries can be treated as well as the main veins resulting in a better cosmetic impact.

Veins below the knee can be treated.

The total procedural time is greatly reduced.

The apparatus is less expensive than laser and RF apparatus.

The procedure is performed in an outpatient setting.

The apparatus automatically assures that the correct amount of sclerosing agent is evenly distributed without requiring the practitioner to carefully monitor the duration of treatment.

FIGS. 5 to 12 illustrate additional vessel flow blocking or occlusion methods and devices according to the present invention.

FIG. 5 illustrates one embodiment of the invention comprising a catheter 110 located within a blood vessel 1. A sponge 112, coupled to a guide wire 114 extending through the catheter 110, is released from the distal end of the catheter 110 by pushing the guide wire distally or by withdrawing the catheter proximally. When the procedure is complete, pulling the guide wire 114 (or pushing the catheter) retrieves the sponge into the catheter whereupon the catheter may be withdrawn. Alternatively, the sponge may comprise an absorbable material, such that it can be left within the vessel following withdrawal of the catheter 110.

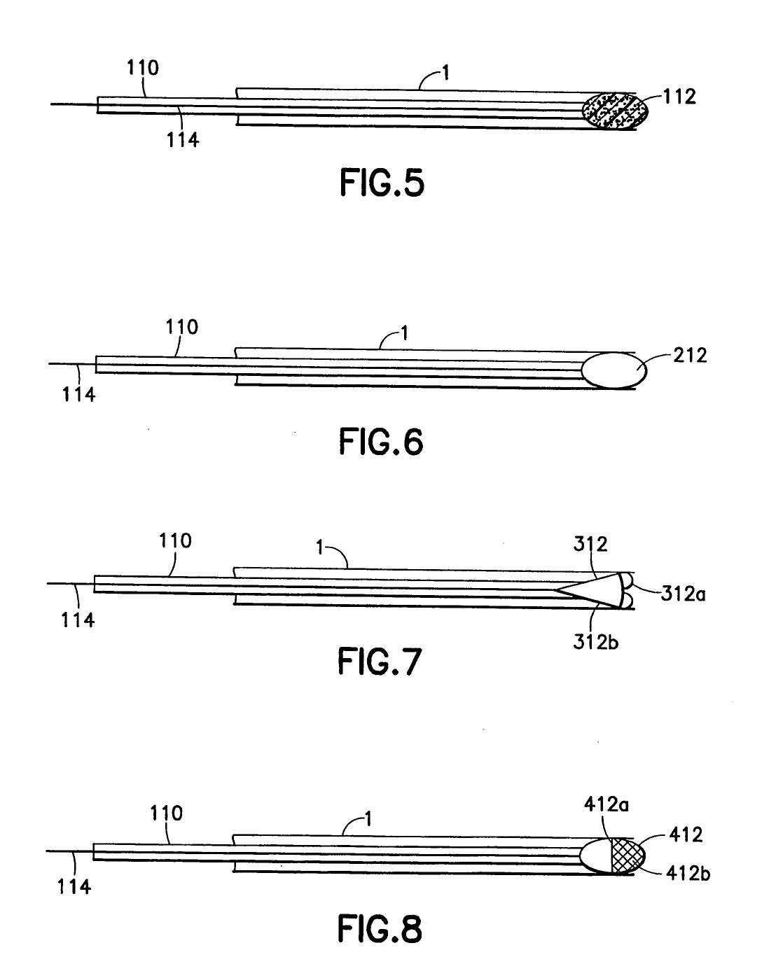

FIG. 6 illustrates one embodiment of the invention comprising a catheter 110 located within a blood vessel 1. A first umbrella occlusion device 212, coupled to a guide wire 114 extending through the catheter 110, is released from the distal end of the catheter 110 by pushing the guide wire distally (or by withdrawing the catheter proximally). When the procedure is complete, pulling the guide wire 114 (or pushing the catheter) retrieves the umbrella 212 into the catheter whereupon the catheter may be withdrawn.

In one embodiment, the umbrella 212 is a structure made of elastic or superelastic wires or struts which are biased to be in an “open,” larger-diameter configuration when there is no external restraint on them, e.g. when released from the catheter. These struts or wires are covered with a membrane or very fine mesh which effectively occludes the flow of blood. Alternatively, the struts can be biased to the closed position, and the structure may be expanded by applying a force to compress the structure axially (by means of two push-pull wires) so as to expand it. (See the previously incorporated co-pending application Ser. No. 10/328,085).

FIG. 7 illustrates one embodiment comprising a catheter 110 located within a blood vessel 1. A second umbrella occlusion device 312, coupled to a guide wire 114 extending through the catheter 110, is released from the distal end of the catheter 110 by pushing the guide wire distally or by pulling the catheter proximally. When the procedure is complete, pulling the guide wire 114 or pushing the catheter distally retrieves the umbrella 312 into the catheter whereupon the catheter may be withdrawn.

In one embodiment, the umbrella 312 includes a tubular inflatable cuff 312a at the distal end of a funnel-shaped membrane 312b. When inflated, the tubular cuff assumes a toroidal shape which expands the membrane to the form of a funnel, contacts the inside wall of the blood vessel and occludes fluid flow. U.S. Pat. No. 5,908,435 describes a catheter device with an inflatable cuff, which when inflated forms a similar funnel-like structure.

FIG. 8 illustrates one embodiment comprising a catheter 110 located within a blood vessel 1. A third umbrella occlusion device 412, coupled to a guide wire 114 extending through the catheter 110, is released from the distal end of the catheter 110 by pushing the guide wire distally or pulling the catheter proximally. When the procedure is complete, pulling the guide wire 114 or pushing the catheter retrieves the umbrella 412 into the catheter whereupon the catheter may be withdrawn.

The umbrella 412 includes an expandable loop of wire 412a coupled to an impervious membrane or film bag 412b. Once extended, the loop and bag expand to fill the lumen of the blood vessel, blocking the flow of fluid.

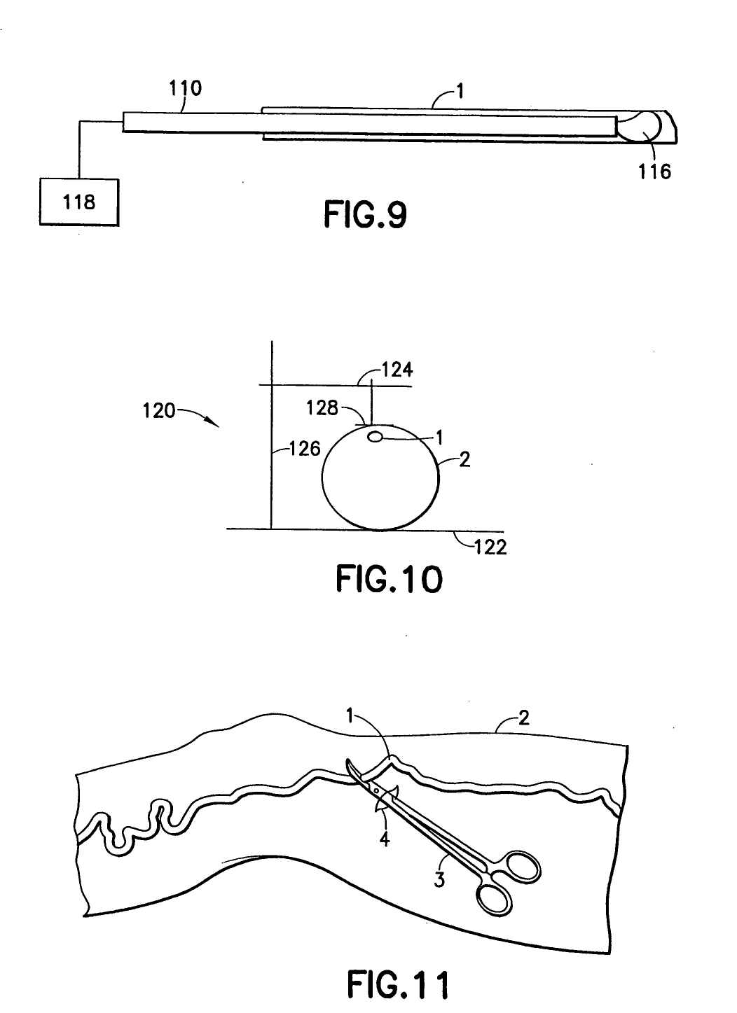

FIG. 9 illustrates one method of occluding a blood vessel 1 by delivering a glue/sealant 116 from a source 118 via a catheter 110 to the site of occlusion. Suitable sealants include butyl-cyanoacrylate, fibrin solution, and other tissue-sealing materials. The sealant is used in a liquid or semi-liquid form to prevent it from embolizing. Once the sealant is applied, the closing of the vein may be assisted by externally applied pressure.

FIG. 10 illustrates in schematic form one embodiment of an apparatus for applying external pressure to a blood vessel 1 in a patient's leg 2. The apparatus 120 generally includes a lower member 122 which is located beneath the patient's leg 2, an upper member 124 located above the patient's leg and coupled to the lower member by a vertical post 126. The upper member 124 may be provided with a pressure pad 128 located directly above the blood vessel 1. Similar apparatus are known for use in closing arterial puncture sites at the groin following arterial access in angioplasty procedures, for example.

In one embodiment, it is possible to occlude the superficial saphenous vein solely by the application of external compression, either by hand or by means of a mechanical assistive device. Examples of compression devices include: inflatable cuffs, inflatable cuffs with means for localizing compression (for example, a rubber bougie or ball), and a mechanical clamping device with a padded “foot.”

FIG. 11 illustrates another embodiment of the invention comprising a method of occluding a blood vessel 1 in a patient's leg 2 with the use of a surgical clamp 3 delivered to the occlusion site via an incision 4. In lieu of a clamp, the practitioner may occlude the blood vessel with a suture (not shown).

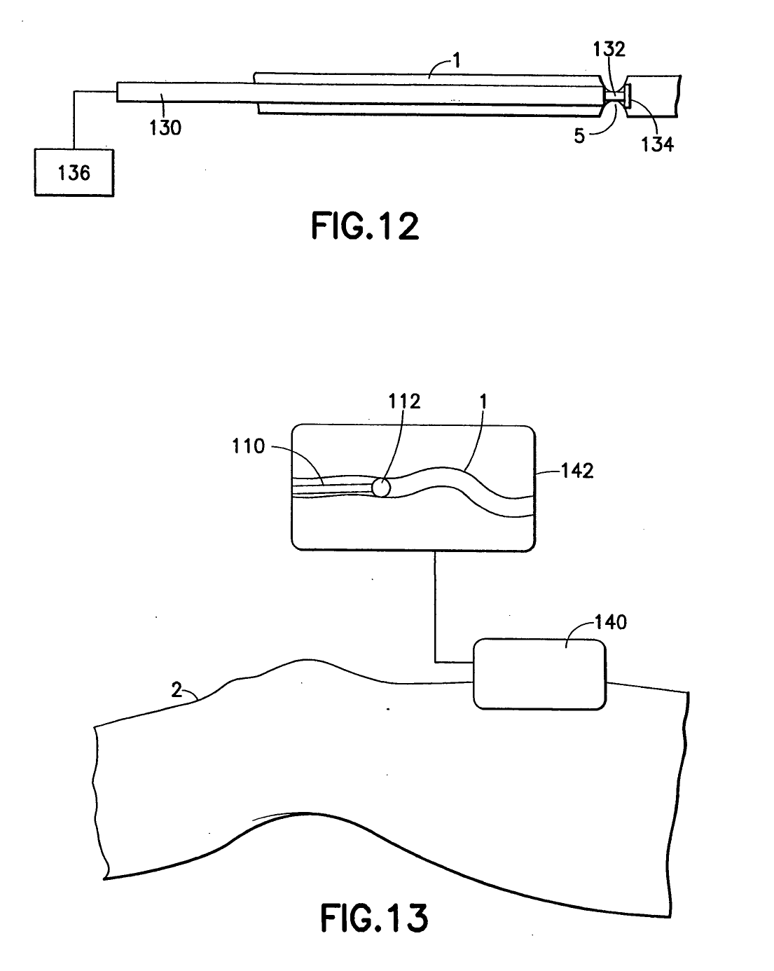

FIG. 12 illustrates a suction device for occluding a blood vessel 1. The suction device includes a catheter 130 having a coaxial extension 132 and a disk 134 which define an annulus at the end of the catheter. The catheter 130 is coupled to a vacuum source 136 and the wall of the blood vessel 1 is drawn into the annulus as illustrated at 5 in FIG. 12.

FIGS. 13 to 18 illustrate methods and apparatus for locating an occlusion device in a blood vessel. Turning now to FIG. 13, an ultrasound device 140 having a display 142 is used to locate a vein 1 in a patient's leg 2. The ultrasound device will also display the location of a catheter 110 and occlusion device 112 within the vein 1.

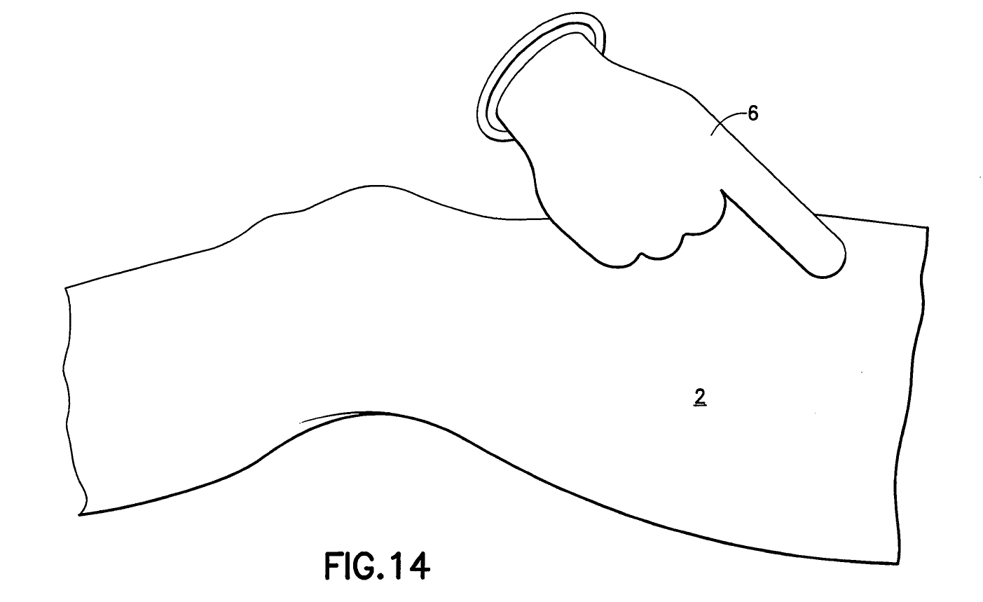

FIG. 14 illustrates a method of locating an occlusion device via palpation. A skilled practitioner can determine the desired location of the occlusion balloon by examination of the leg. The distal end of the occlusion catheter can then be located by palpation, especially if there is a distal bulb or other feature on the occlusion catheter. FIG. 14 shows the practitioner's hand 6 palpating the patient's leg 2.

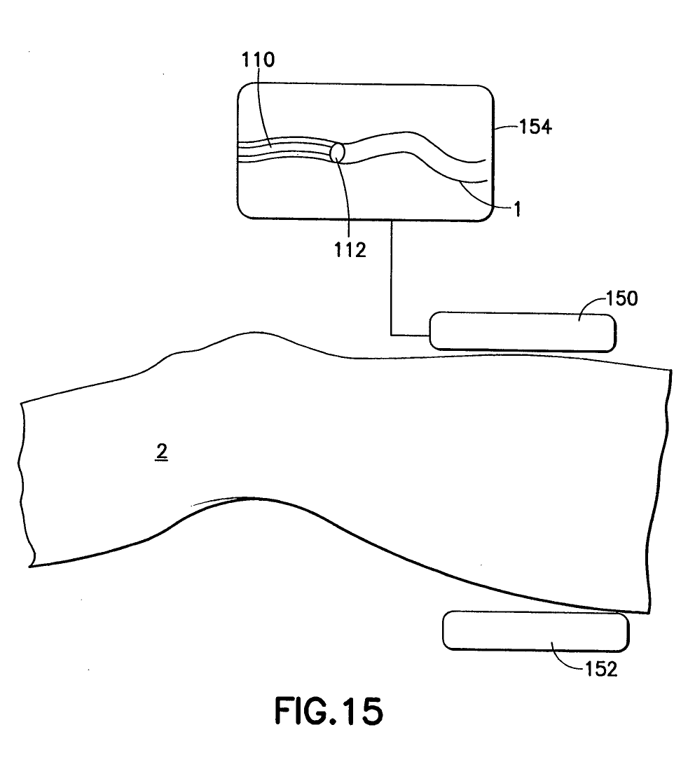

FIG. 15 illustrates a method of locating an occlusion device using radiation imaging such as fluoroscopy or magnetic resonance imaging. A detector 150 is placed over the patient's leg 2 and a source of radiation 152 is placed beneath the leg 2. The detector 150 is coupled to a display 154 which illustrates the patient's blood vessel 1, the catheter 110 and the occlusion device 112.

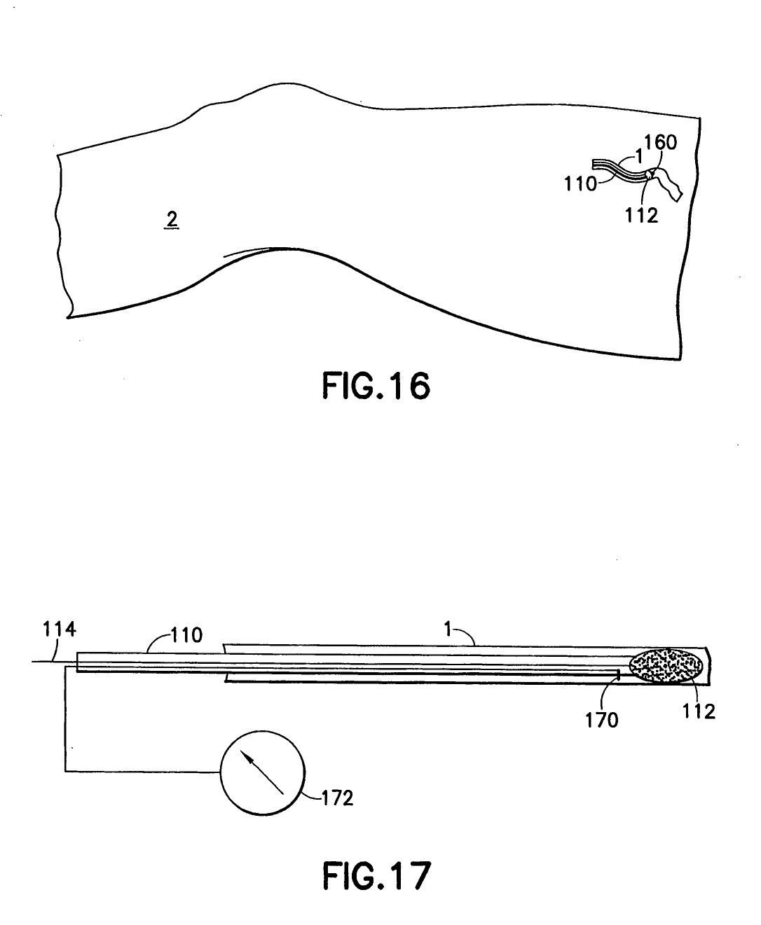

FIG. 16 illustrates a catheter 110 having an occlusion device 112 and a light source 160 (e.g., an LED or fiber optic tip) adjacent the occlusion device 112. Once the desired location of the occlusion balloon in vein 1 has been determined by examining the leg 2, the occlusion device is easily located by the light emitted from the light source 160 which is bright enough to be seen through the patient's skin.

FIG. 17 illustrates a catheter 110 with an occlusion device 112 located within a blood vessel 1. The catheter 110 is provided with a pressure sensor 170 which is coupled to a pressure gauge 172. The pressure in the femoral vein is lower than the pressure in the saphenous vein. Therefore, by monitoring the fluid pressure at the distal end of the occlusion catheter 110, it is possible to determine when the pressure sensor moves from the saphenous vein into the junction of the saphenous vein and the femoral vein. If actuation of the occlusion device in the saphenous vein is desired, the pressure sensor can then be withdrawn proximally into the saphenous vein proximal of the femoral vein (as indicated by a resulting increase in blood pressure), and the occlusion device actuated therein.

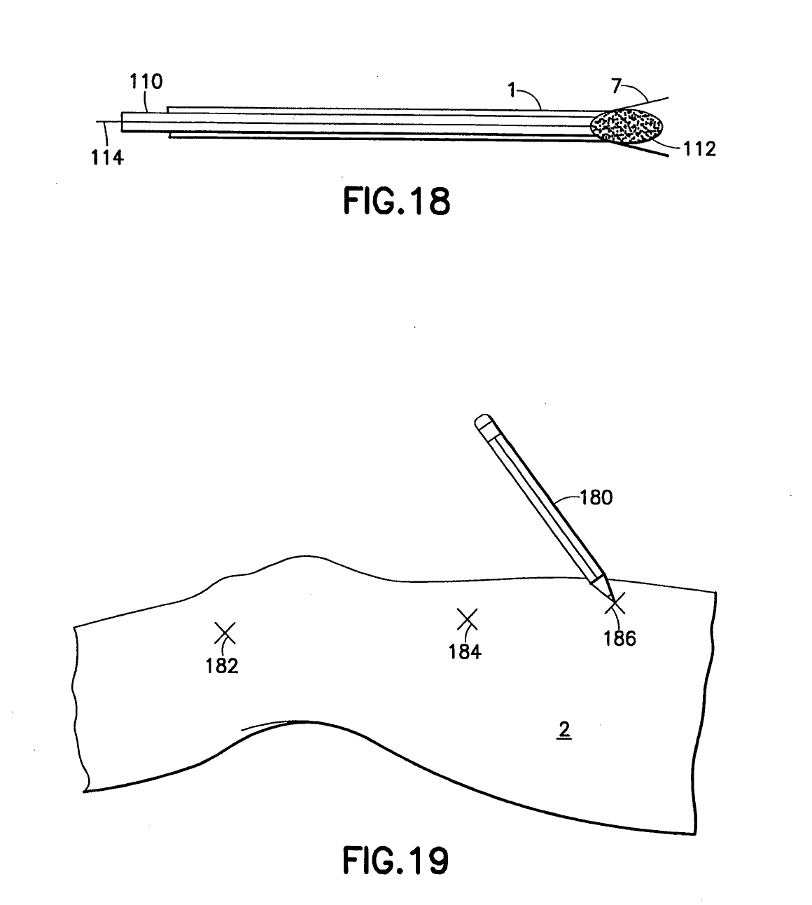

FIG. 18 shows a catheter 10 having an occlusion device 112 coupled to deployment means 114. According to a method of the invention, the occlusion device is located at the desired site by first passing it into the femoral vein 7, then deploying it, then pulling it back until it “wedges” against the junction of the saphenous vein 1. In the case of an occlusion balloon, by deflating the balloon, withdrawing it a short distance (1-2 cm), and re-inflating it, the occlusion balloon can be correctly located at the desired location in the saphenous vein.

According to the methods of the invention, an additional bolus of treating agent is optionally dispensed when the treating catheter passes a tributary blood vessel. FIGS. 19 to 24 illustrate methods for locating tributary blood vessels which include pre-marking the patient's skin. FIG. 19 shows the first step in which a marker 180 is used to make fiducial marks 182, 184, 186 on the surface of the leg 2 in registration with the side branches of the saphenous vein. These marks are made prior to the procedure of treating the blood vessels with the aid of ultrasound or other imaging (e.g., x-ray, MRI, or trans-illumination). Once these marks have been placed, the position of the catheter can be controlled by any of the following methods.

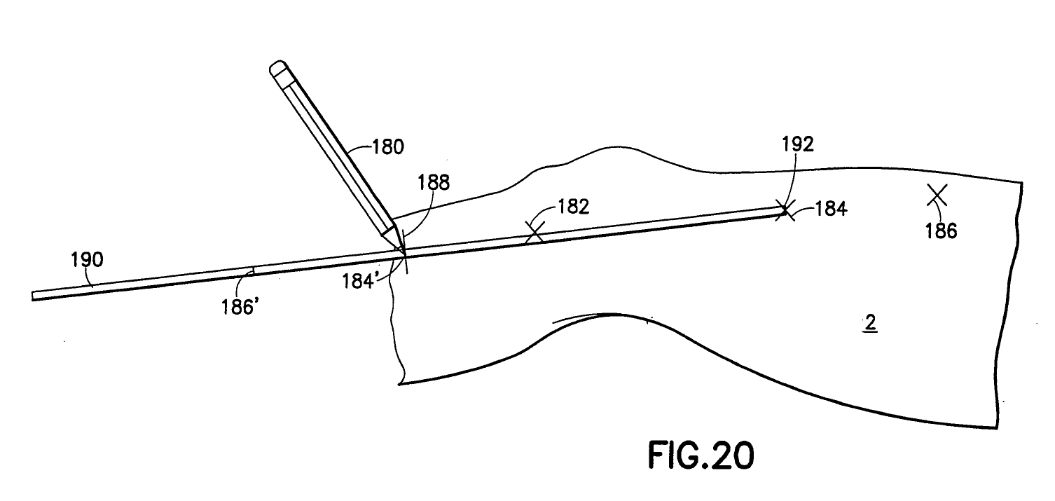

FIG. 20 shows a treating catheter 190 placed on top of the patient's leg 2. Fiducial marks are placed on the catheter by aligning the catheter on the outside of the leg along the path of the saphenous vein. The treating end 192 of the catheter 190 is positioned at each of the side-branch marks 186, 184, 182, etc. A corresponding fiducial mark 186′, 184′, etc. is placed on the catheter where the catheter will exit the venipuncture 188. In this way, the practitioner creates on the outside of the catheter an array of fiducial marks such that during the procedure whenever one of these marks is coincident with the venipuncture (or any other convenient index mark), the distal end 192 of the treating catheter 190 is adjacent to one of the side branches. Alternatively, in embodiments where a “pull wire” is used to retract the catheter, these marks can be applied to the pull wire.

Another (unillustrated) method of utilizing the pre-markings on the patient's leg is to use a catheter with a light source at its treating end such as the light source shown in FIG. 16. When the light source is seen under the side branch mark, additional treating agent is optionally dispensed. Still another (unillustrated) method of utilizing the pre-markings on the patient's leg is to palpate the location of the treating end of the catheter such as shown in FIG. 14. In this method, the treating catheter is preferably provided with a bulb or bougie which can be felt through the patient's skin. Thus, when palpation at or adjacent to pre-markings indicates location of the distal end of the catheter thereat, additional treating agent is optionally dispensed to treat the tributary blood vessel(s).

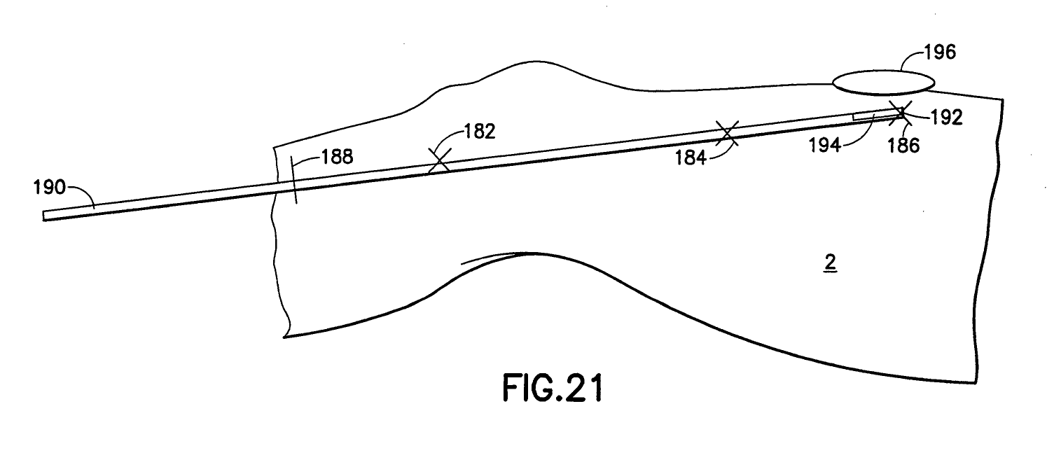

FIG. 21 illustrates a first method of utilizing the pre-markings on the patient's leg with a magnetic follower. Here the treating catheter 190 is provided with a magnet 194 at its treating end 192. A magnetic follower 196 is placed on the patient's leg 2. The follower rolls or slides along the surface of the leg showing the location of the treating end of the catheter. Whenever the follower passes over a pre-marking, additional treating agent is optionally dispensed to treat the tributary blood vessel(s).

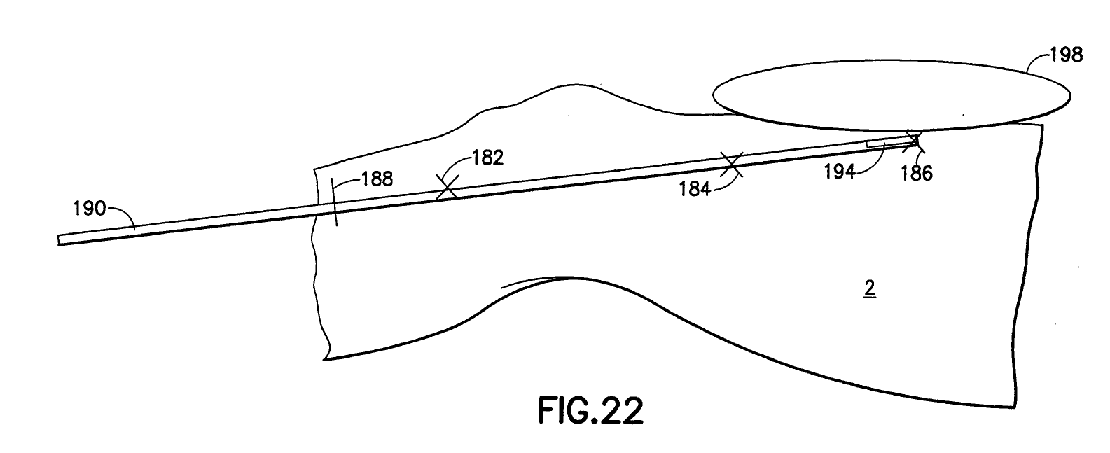

FIG. 22 illustrates another method of utilizing the pre-markings on the patient's leg with a magnet located on the treating end of a catheter. This method uses a transparent magnetic visualization screen 198 which contains iron filings. The screen is held over the markings on the patient's leg and when the magnet 194 on the catheter 190 passes under the screen, the iron filings show its movement. Whenever the screen indicates that the treating end of the catheter is located at a pre-marking, additional treating agent is optionally dispensed to treat the tributary blood vessel(s).

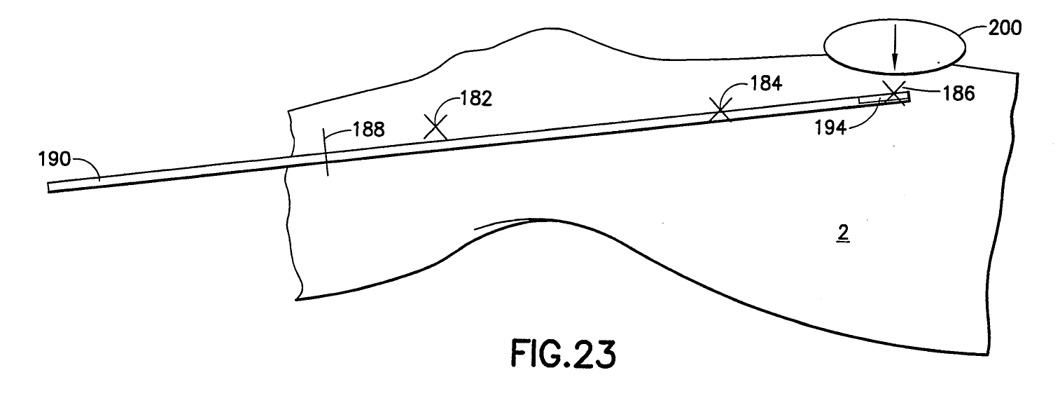

FIG. 23 illustrates another method of utilizing the pre-markings on the patient's leg with a magnet located on the treating end of a catheter. This method uses a hand held magnet detector such as a compass 200. The compass 200 is placed by the markings on the patient's leg and the compass needle indicates the passage of the magnet 194 on the treating end of the catheter 190. Whenever the magnet passes under a pre-marking, additional treating agent is optionally dispensed to treat the tributary blood vessel(s).

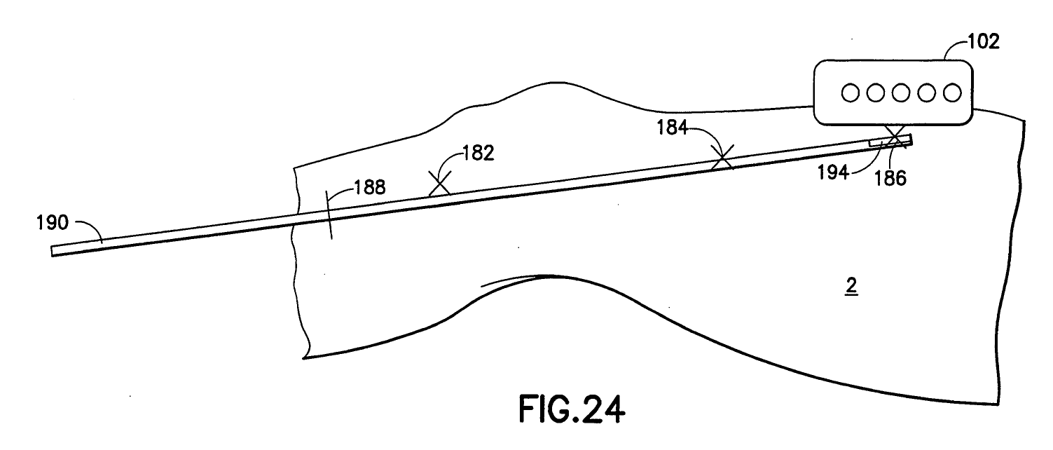

FIG. 24 illustrates another method of utilizing the pre-markings on the patient's leg with a magnet located on the treating end of a catheter. This method uses a hand held magnet detector such as an electronic device 202 having a plurality of LEDs which light as a magnet passes. The device 202 is placed by the markings on the patient's leg and the LEDs indicate the passage of the magnet 194 on the treating end of the catheter 190. Whenever the LED device indicates that the magnet is located under a pre-marking, additional treating agent is optionally dispensed to treat the tributary blood vessel(s).

The invention also contemplates methods of locating the treating end of a catheter at tributaries without pre-marking via different types of imaging such as ultrasound such as described above with reference to FIG. 13, fluoroscopic imaging such as described above with reference to FIG. 15, and a bright light coupled to the treating end of the catheter such as described above with reference to FIG. 16.

FIG. 25 illustrates the use of an external light source 300 which is used to direct light onto a region 302 of the patient's leg 2. The light source 300 is preferably an infrared (IR) light source, and an IR viewing device 304 (such as IR goggles) is used to determine the location of the treating end 192 of the catheter 90 at tributaries 1 a in vein 1.

FIGS. 26 to 32 illustrate various catheter devices according to the invention.

Turning now to FIG. 26, an occlusion catheter 400 has an inflatable balloon 402 coupled to its distal end and an atraumatic floppy guide wire tip 404 coupled to the distal end of the balloon.

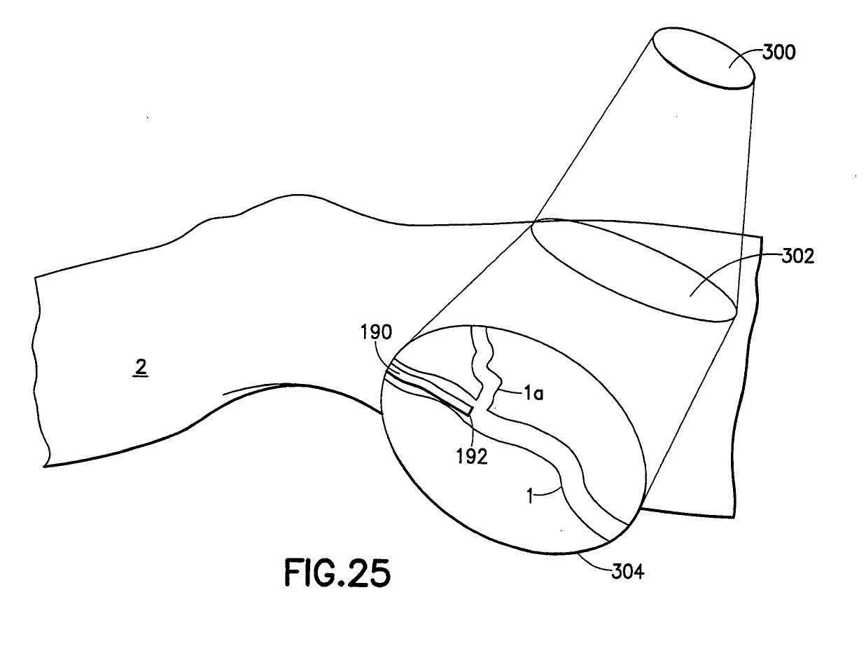

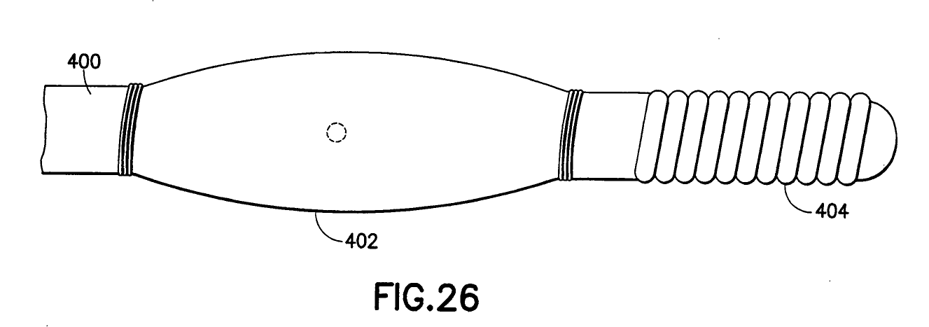

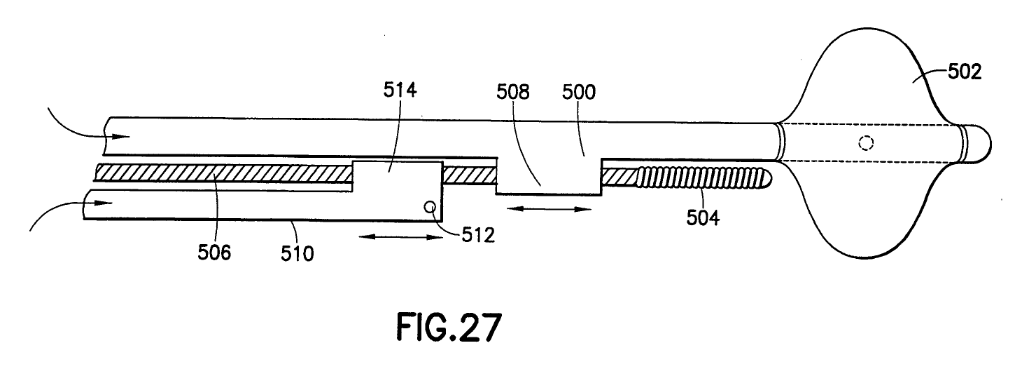

FIG. 27 illustrates a dual monorail system which includes an occlusion catheter 500 having an inflatable balloon 502 and a first monorail coupling 508. A guide wire 506 having an atraumatic tip 504 is arranged to pass through the monorail coupling 508. A treating catheter 510 having a distal fluid outlet 512 is also provided with a monorail coupling 514 through which the guide wire 506 also passes. From the foregoing, those skilled in the art will appreciate that the assembly is configured as shown but with the balloon 502 deflated. The guide wire is delivered to the site where the occlusion balloon is to be inflated. The occlusion catheter and treating catheter are delivered over the guide wire until the balloon is at the desired location. The balloon is then inflated. Treating fluid is then dispensed as the catheter 510 is withdrawn over the guide wire. At the locations of tributaries, additional treating fluid is optionally dispensed.

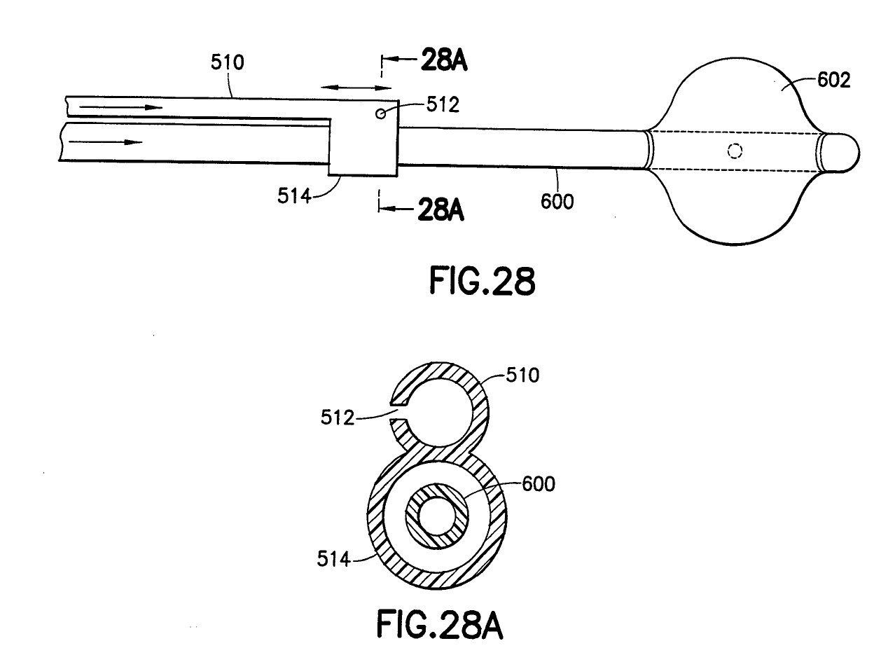

FIGS. 28 and 28A illustrate a single monorail system which includes an occlusion catheter 600 having an inflatable balloon 602 at its distal end and a treating catheter 510. The treating catheter 510 has a drug dispensing port 512 and a monorail coupling 514 through which the occlusion catheter 600 extends. From the foregoing, those skilled in the art will appreciate that the assembly is configured as shown but with the balloon 602 deflated. The occlusion catheter 600 and the treating catheter 510 are delivered through the blood vessel until the balloon is at the desired location. The balloon is then inflated. Treating fluid is then dispensed as the catheter 510 is withdrawn over the catheter 600. At the locations of tributaries, additional treating fluid is optionally dispensed.

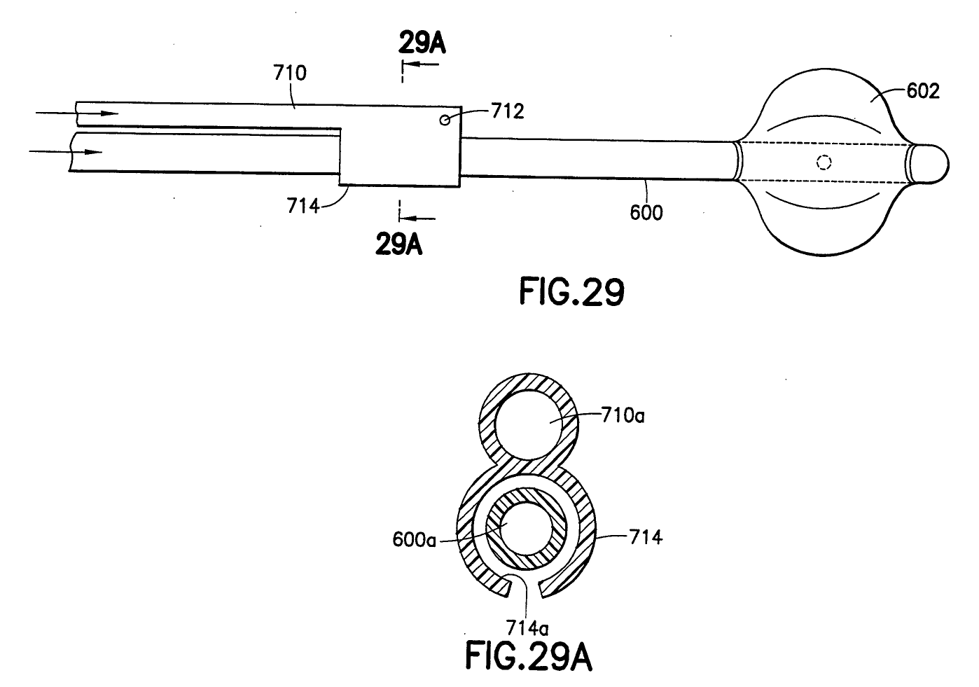

FIGS. 29 and 29A illustrate a clip-on monorail system which includes an occlusion catheter 600 having an inflatable balloon 602 at its distal end and a treating catheter 710. The treating catheter 710 has a drug dispensing port 712 and a clip-on monorail coupling 714 through which the occlusion catheter 600 extends. From the foregoing, those skilled in the art will appreciate that the assembly is configured as shown but with the balloon 602 deflated. The occlusion catheter 600 and the treating catheter 810 are delivered through the blood vessel until the balloon is at the desired location. The balloon is then inflated. Treating fluid is then dispensed as the catheter 710 is withdrawn over the catheter 600. At the locations of tributaries, additional treating fluid is optionally dispensed. FIG. 29A illustrates the inflation lumen 600a of the occlusion catheter 600, and the drug delivery lumen 710a of the treating catheter 700. The inner surface 714a of the clip-on monorail coupling 714 is preferably a lubricous contact surface.

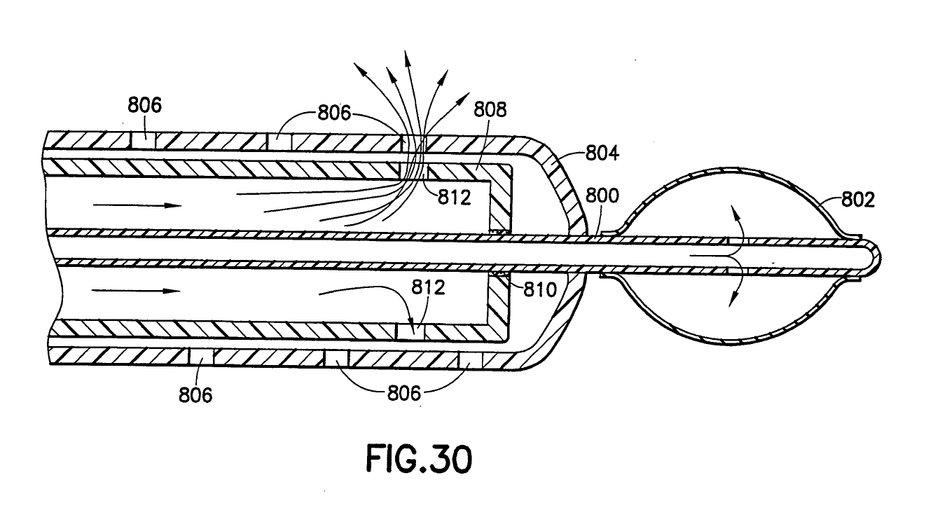

FIG. 30 illustrates an occlusion and drug delivery system which includes an occlusion catheter 800 having an inflatable balloon 802 at its end. A first coaxial outer catheter 804 extends over the occlusion catheter 800 and is preferably coupled to it. The catheter 804 has a plurality of perforations 806 along its length. A second coaxial inner catheter 808 extends over and is movable along the occlusion catheter 800 within the first coaxial outer catheter 804. The second coaxial inner catheter 808 is preferably provided with an annular fluid seal 810. The second coaxial inner catheter 808 is provided with at least one radial fluid outlet 812 which aligns with the perforations 806 in the first coaxial outer catheter 804 as the catheter 808 is moved along the catheter 800. From the foregoing, those skilled in the art will appreciate that the assembly is configured as shown but with the balloon 802 deflated. The three catheters are delivered through the blood vessel until the balloon is at the desired location. The balloon is then inflated. Treating fluid is then dispensed as the coaxial inner catheter 808 is withdrawn over the catheter 800 but with the coaxial catheter 804 in place.

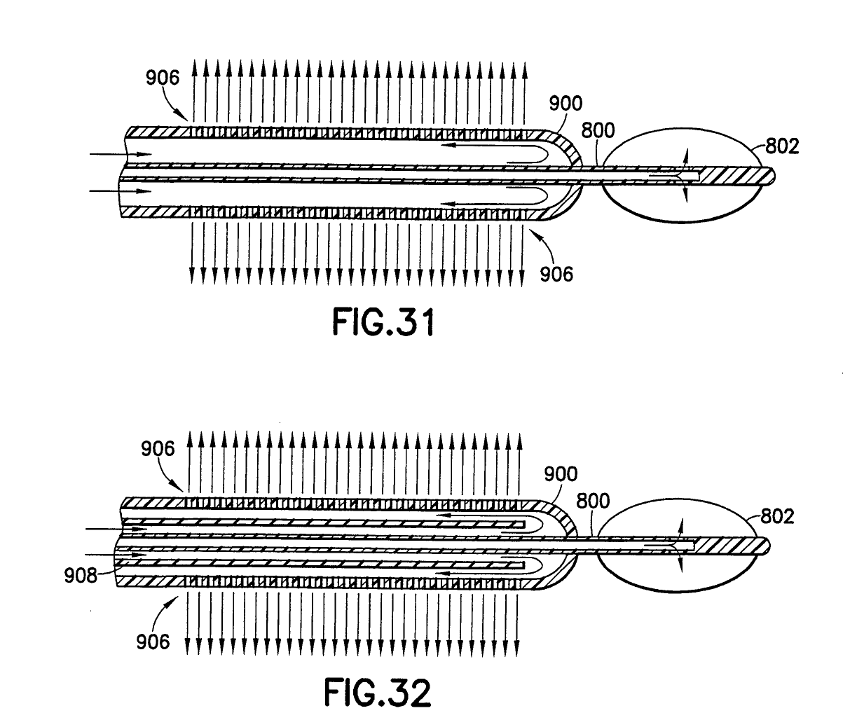

FIG. 31 illustrates an occlusion and drug delivery system which includes an occlusion catheter 800 having an inflatable balloon 802 at its end. A coaxial outer catheter 900 extends over the occlusion catheter 800 and is preferably coupled to it. The catheter 900 has a plurality of very small perforations 906 along its length. In use, the catheters are delivered through the blood vessel until the balloon is at the desired location. The balloon is then inflated. Treating fluid is then dispensed into the annular space between the catheters as shown by the arrows in FIG. 31. As the annular space fills, sufficient pressure is reached so that the fluid weeps out of the small perforations 906 along the length of the catheter 900.

FIG. 32 illustrates a second embodiment of a weeping catheter system. This arrangement is similar to the arrangement shown in FIG. 31 but for the addition of an annular baffle 908 between the catheter 800 and the catheter 900. The baffle prevents release of treating fluid through the perforations 906 until the fluid has first reached the distal end of the catheter system and then is redirected proximally in an annular space defined by the baffle 1008 and the weeping catheter 900.

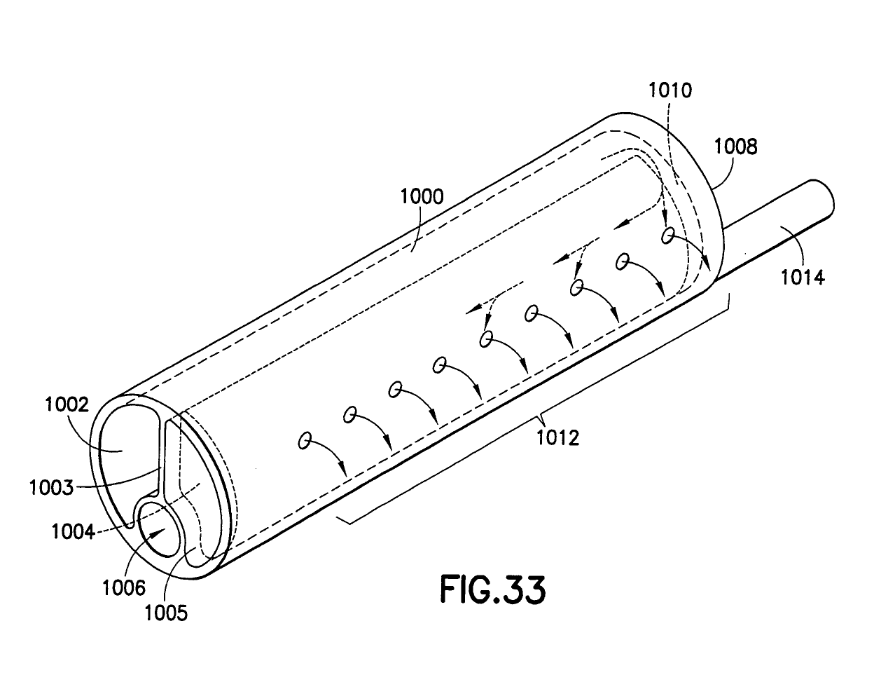

FIG. 33 illustrates a portion of a third embodiment of a weeping catheter 1000. The catheter has three lumena 1002, 1004, and 106. The lumena 1002 and 1004 are larger than the lumen 1006 and are separated by a wall 103. The ends of the lumena 1002 and 1004 are closed at 1008, but wall 1003 is stopped proximal of wall 1008 such that a fluid passage 1010 is formed to couple distal portions of the lumena 1002 and 1004. A plurality of perforations 1012 are provided along the length of the catheter 1000 in fluid communication with the lumen 1004. The proximal portion 105 of the lumen 104 is sealed. As shown in FIG. 33, a tubular extension 1014 is provided at the distal end of the catheter. This extension 1014 is in fluid communication with the lumen 1006 and is used to inflate a balloon not shown in this Figure. From the foregoing, those skilled in the art will appreciate that treating fluid delivered through lumen 1002 will travel to the end of the catheter and pass through the passage 1010 into the lumen 1004 where it will travel proximally past all of the perforations 1012 weeping out of the catheter.

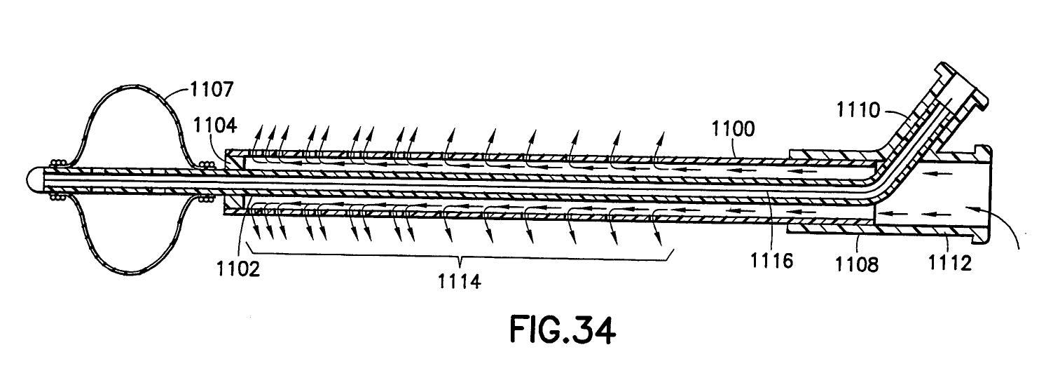

FIG. 34 shows a weeping catheter 1100 with a coaxial balloon inflation catheter 1102. The distal end of the catheter 1100 is provided with an annular seal 1104 between it and the inflation catheter 1102. The distal end of the inflation catheter 1102 is provided with an inflatable balloon 1107. The proximal end of the weeping catheter 1100 is coupled to a fluid coupling port 1108 having a side port 1110 and a main port 1112. The proximal end of the inflation catheter 1102 is coupled to the side port 1110. The weeping catheter 1100 has a plurality of perforations 1114 along at least a portion of its length. Preferably, a support wire 1116 is disposed inside the inflation catheter 1102 from its proximal end to its distal end to provide desired stiffness. From the foregoing, those skilled in the art will appreciate that fluid dispensed through the side port 1110 will inflate the balloon 1107 and treating fluid dispensed through the main port 1112 will weep through the perforations 1114.

According to the invention, the weeping catheters described above with reference to FIGS. 30 to 34 may be provided with different perforation configurations. The diameters of the perforations may be constant or variable. The spacing of the perforations may be constant or variable. Perforations may be provided in groups which are evenly spaced or variably spaced. The number of perforations per group may be constant or variable. These different configurations are chosen so as to provide either equal or biased infusion along the treating length of the weeping catheter.

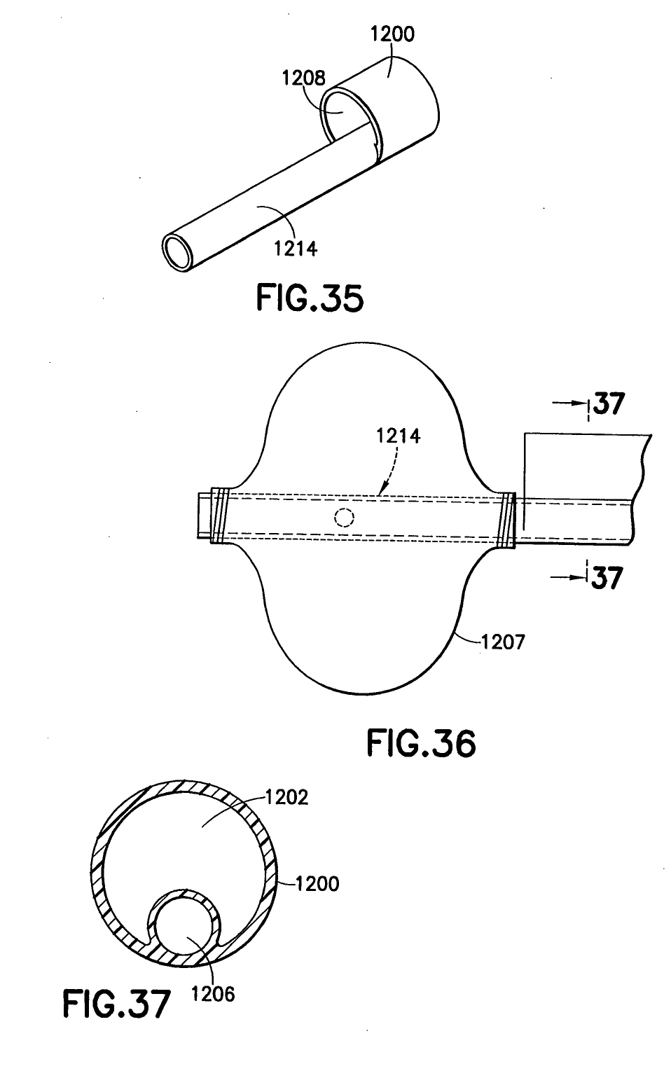

FIGS. 35 to 37 illustrate a portion of a weeping catheter 1200 which can be considered to be a combination of the catheters 1000 and 1100. In this embodiment, the inflation catheter (or lumen) 1206 is not coaxial with the weeping catheter 1200 and the infusion space (or lumen) 1202 is not annular as in the catheter 1100. However, the distal end 1214 of the inflation catheter is provided with an inflatable balloon 1207 which is substantially similar to the arrangement shown in FIG. 34. The distal end 1208 of the infusion space 1202 is sealed and a plurality of perforations into the infusion space are provided along the treating length of the catheter 1200 as described above with reference to the other weeping catheter embodiments, but not shown in FIGS. 35 to 37.

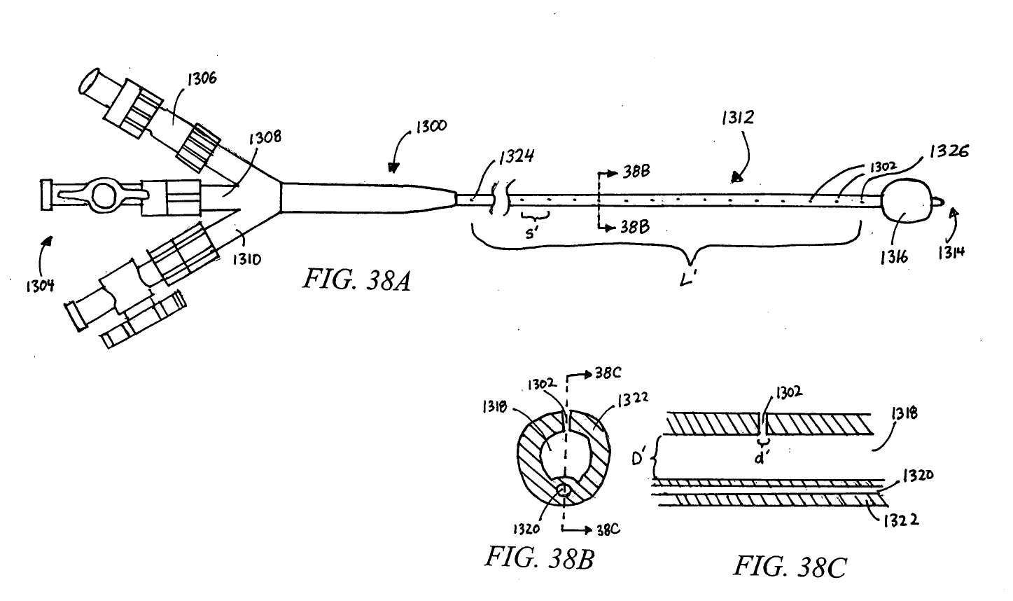

FIGS. 38A to 38C depicts one embodiment of the invention comprising an infusion catheter 1300 capable of generally simultaneous infusion of the treatment agent through a plurality of holes 1302 located along the length of the catheter 1300. The catheter 1300 comprises a proximal end 1304 with at least one access port 1306, 1308, 1310, a catheter body 1312, and a distal end 1314 with a blood vessel occluder 1316.

In one embodiment, each access port 1306, 1308, 1310 is in fluid communication with a lumen running generally along the length of the catheter body. In some embodiments, a lumen may be in fluid communication with multiple access ports. In one embodiment, at least one access port 1306 is in fluid communication with an infusion lumen allow infusion of a treatment agent into the catheter 1300 and out through the holes 1302 of the catheter body 1312. In one embodiment, one access port 1310 and lumen 1320 is provided to allow manipulation of the blood vessel occluder 1316 from the proximal end 1304 of the catheter 1300. The inflation lumen 1320 may be integral with the outer catheter wall 1322 or be defined within a separate tubular wall (not shown) within the infusion lumen 1318.

In one embodiment, the catheter 1300 is configured so that the fluid elution from the holes 1302 generally occurs in a particular predetermined pattern when the fluid is injected through the catheter 1300 at a specific viscosity and pressure or pressure range. In one embodiment of the invention, the pattern of fluid elution is determined by at least one of several factors, including but not limited to: 1) the hydraulic diameter D′ of the infusion lumen of the catheter; 2) the hydraulic diameter d′ of each elution hole; 3) the spacing s′ between each elution hole; 4) the overall treatment length L′ of the catheter; 5) the viscosity of the agent used for treatment; and 6) the compressibility of the treatment agent. The term “hydraulic diameter”, as used herein, shall be given its ordinary meaning and shall also include the equivalent diameter of a structure when estimating pressure loss or head loss in non-circular lumena using data made for circular lumena. The term “treatment length” as used herein shall mean the portion of the catheter generally from about the most proximal elution hole 1324 to about the most distal elution hole 1326.

In one embodiment, the fluid distribution from the catheter 1300 is generally even along the treatment length of the catheter 1300. In another embodiment, the pattern of fluid distribution from the catheter 1300 provides for increased elution of agent at the distal end 1314 of the treatment length. The change in elution along the treatment length may be a gradual ramp or stepped. In another embodiment, the fluid distribution pattern provides greater elution at the proximal end 1304 of the treatment length. In another embodiment, the catheter 1300 provides a customized distribution pattern adapted to provide increased flow at one or more locations along the treatment length which is adapted to correspond to the location of the venous tributaries when the occluder has been positioned as described herein. In another embodiment, the catheter 1300 provides a customized distribution pattern adapted to provide increased flow at the venous tributaries and about the saphenofemoral junction. One skilled in the art will understand that the catheter may be configured for any of a variety of elution or distribution patterns.

The diameter D′ of the infusion lumen 1318 of the catheter 1300 generally ranges from about 0.03″ to about 0.20″. In certain embodiments, the diameter d′ ranges from about 0.05″ to about 0.09″. In one embodiment, the diameter d′ is about 0.072″.

The overall treatment length L′ of the catheter generally ranges from about 10 cm to about 175 cm. In certain embodiments, the treatment length L′ is within the range of from about 20 cm to about 100 cm. In another embodiment, the treatment length L′ is within the range of from about 20 cm to about 44 cm.

The viscosity at body temperature of the treatment agent is generally within the range of from about 1.00E-04 (lb*s/in\hat{ }2) to about 1.00E-08 (lb*s/in\hat{ }2). In certain embodiments, the viscosity of the treatment agent is within the range of from about 1.00E-06 (lb*s/in\hat{.}2) to about 1.00E-08 (lb*s/in\hat{ }2). In one embodiment, the viscosity is about 1.74E-07 (lb*s/in\hat{ }2). Viscosities outside of the foregoing ranges may also be used, taking into account the pore sizes, infusion lumen length and diameter, as long as the desired delivery performance (e.g. delivery rate) is achieved. Sclerosing agents used for treating veins are generally incompressible, but compressible agents may also be used.

In one embodiment, the spacing s′ between the elution holes 1302 ranges from about 0.01 cm to about 10 cm. The spacing s′ between the elution holes 1302 may range from about 0.50 cm to about 5 cm. In other embodiments, the spacing s′ between the elution holes 1302 is about 0.50 cm to about 3 cm. In another embodiment, the spacing s′ between the elution holes 1302 is about 0.50 cm to about 2 cm.

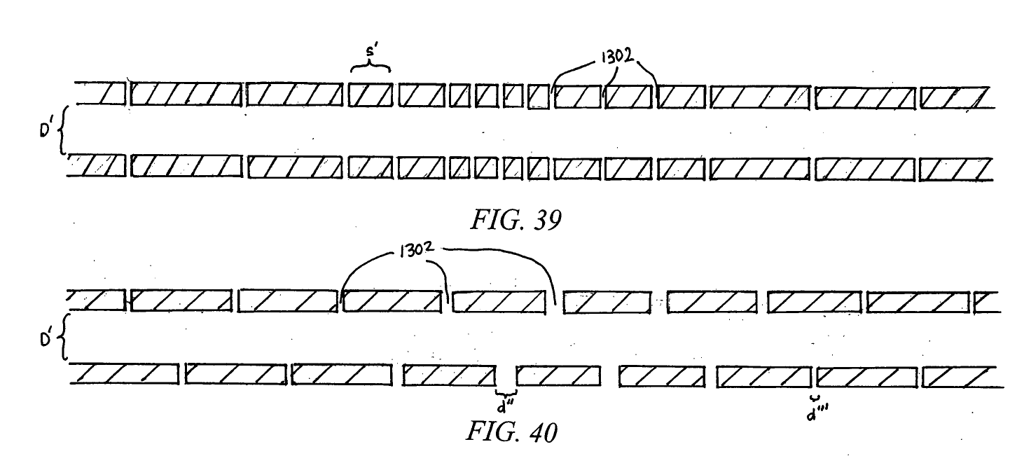

FIG. 39 shows that the spacing between the elution holes 1032 may vary along the length of the catheter. Portions of the catheter with increased spacing s″ may exhibit a reduced elution rate compared to portions of the catheter with decreased spacing s″′, for a given hole diameter. Variations in the spacing of elution holes may be used to achieve variations in the elution patterns of the catheter. The elution pattern is defined by the elution rates at different segments of the infusion catheter. For example, an even elution pattern generally has similar elution rates along the all the segments catheter, while a distal elution pattern provides increased elution rate in at least one segment of the catheter located distally. Increased elution in a particular zone or region of the catheter may be provided by increasing the total cross sectional area of the elution holes in that region, such as by either increasing the elution hole density or the elution hole diameters or both in that region.

The diameter d′ of the elution holes 1032 may be selected for the desired elution pattern by considering the catheter and sclerosing agent characteristics described previously and the pressure drop-off along the catheter length. In one embodiment of the invention, the elution hole diameter is about 0.001″ to about 0.015″. In another embodiment, the elution hole diameter is about 0.002″ to about 0.010″. In one embodiment, based upon a 6-French catheter with a length greater than 40 cm, elution hole spacing between 1 cm and 2 cm and sclerosing agent characteristics described previously, an elution hole diameter of about 0.004″ or less is capable of providing a generally uniform fluid elution along the length of the infusion catheter 1300. Other elution hole diameters may also be used, depending on the desired elution pattern for the infusion catheter and the catheter and sclerosing agent characteristics used.

FIG. 40 shows that the diameters of the elution holes 1300 need not be uniform. Larger elution hole diameters d″ will generally have a higher elution rate than smaller elution hole diameters d″′, but other factors, such as the pressure drop-off along the catheter, will also effect the relative elution rates between the elution holes. In one embodiment of the invention, elution holes located in the distal portion of the catheter generally have a greater diameter than elution holes in the more proximal portions of the catheter to compensate for the pressure drop along the length of the delivery zone and produce a relatively constant delivery profile. The cross sectional shape of the elution holes can be circular, oval, square, triangular or any polygonal or closed shape. The cross sectional shape of the elution holes need not be uniform throughout the longitudinal length of the elution hole. In one embodiment, variations in elution hole diameter and elution hole spacing are used to alter the elution pattern.

In one embodiment of the invention, the diameters d′ of the elution holes 1302 each have an effective hydraulic diameter less than the fluid distribution lumen D′ that connects the elution holes 1302. In a further embodiment, the total fluid resistance of the plurality of elution holes 1302 is generally equal or greater than fluid resistance of the infusion lumen 1318 or lumena of the catheter. In still a further embodiment of the invention, the total fluid resistance of the plurality of elution holes 1302 is substantially greater than the fluid resistance of the catheter infusion lumen 1318. By providing elution holes 1032 with a total fluid resistance substantially greater than the infusion lumen 1318, uniform elution along the catheter 1300 may be achieved. The total fluid resistance of the infusion lumen should generally be less than about 80 percent of the total fluid resistance of the elution holes, and in certain devices less than about 50 percent of the total fluid resistance of the elution holes. The hydraulic diameters of the elution holes 1302, however, are not limited to consideration of the factors described above.

The wall thickness of the infusion catheter 1300 may also contribute to the total fluid resistance of the plurality of elution holes 1032. The wall thickness essentially corresponds to the length of a capillary tube, creating resistance to flow which may at least theoretically be determined by well known relationships such as Poiseuille's law. For example, a 6-French catheter made of Versamid® polyamide resin may have a wall thickness within the range of about 0.006″ to 0.015″. Where the elution holes have a hydraulic diameter of about 0.004″ or less, the wall thickness, which defines the length of the elution holes 1302, may contribute to the fluid resistance of the elution hole 1302. In one embodiment of the invention, the catheter has a wall thickness of about 0.003″ to about 0.100″. In another embodiment, the catheter has a wall thickness of about 0.004″ to about 0.060″. In another embodiment, the catheter has a wall thickness of about 0.005″ to about 0.030″. In still another embodiment, the catheter has a wall thickness of about 0.004″ to about 0.020″.

The elution rate at a given segment of the catheter is affected by spacing s′ and hole diameter d″ of elution holes 1302, the distance of the segment from the proximal end of the catheter, as well as the spacing s′ and diameter d′ of the other catheter segments. One skilled in the art will understand that these characteristics, and other characteristics described previously, can be altered to achieve a different elution pattern.

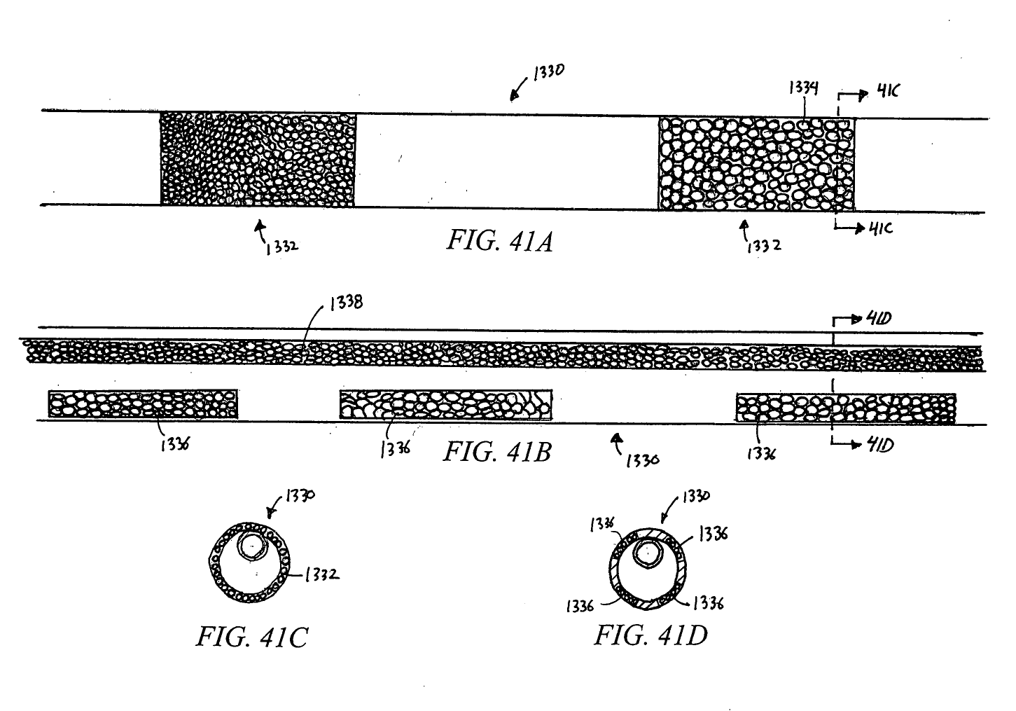

FIGS. 41A to 41D illustrates one embodiment of the invention, where the medicament is eluted from the catheter 1330 through at least one catheter portion comprising a porous or permeable region 1332. The porous region comprises a plurality of small openings 1334 through which the medicament may elute. In one embodiment, the region has a porosity of about 2 microns to about 40 microns. In another embodiment, the region has a porosity of about 4 microns to about 20 microns. In another embodiment, the region has a porosity of about 6 microns to about 12 microns. In one embodiment, the region has a porosity of about 8 microns which is preferably capable of resisting clogging from blood constituents. The porosity of the porous or permeable regions need not be uniformly porous between regions or within the same region.

A porous portion 1332 may comprise a full circumference of catheter, as shown in FIGS. 41A and 41C, or a portion of the circumference, as shown by segments 1336, 1338 in FIGS. 41B and 41D. The infusion catheter may comprise a single porous portion, multiple contiguous porous portions or multiple porous portions separated by non-porous portions. Multiple porous portions may be arranged serially along the longitudinal length of the catheter as shown by segments 1336, in parallel where the porous portions are longitudinal strips 1338 along the length of the catheter, or any combination thereof. In another embodiment, a combination of porous regions and elution holes may be used to provide the desired elution pattern for the catheter. The porous material may include, but is not limited to, a ceramic, ultrahigh molecular weight polyolefin, a perforated polymer film, porous or microporous membranes, polyethersulfone, TYVEK (spun-bonded polyethylene), GORTEX (expanded PTFE), woven or knit mesh or fabric, and other porous materials.

In one embodiment of the invention, a system for controlling or altering the flow of medicament at an elution hole, a series of elution holes, or a porous region is provided. Multiple elution control systems may be used in the same catheter to provide control over multiple portions of the catheter. A control system may also be capable of protecting the elution hole from clogging with blood components by exposing the elution hole only during periods of desired elution and protecting the elution holes at other times. Several embodiments of the control system are described below.

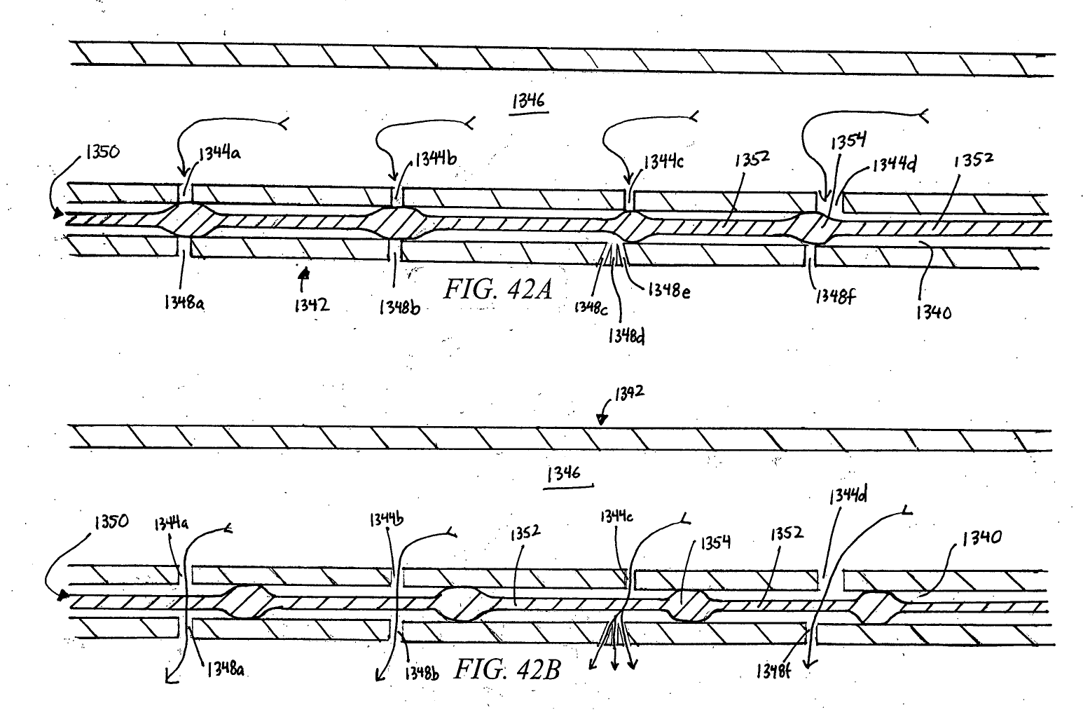

FIGS. 42A and 42B show one embodiment of the invention, where the fluid control system comprises a separate or side lumen 1340 generally along the length of the infusion catheter 1342. At least one inner hole 1344a-1344d is provided between the infusion lumen 1346 and side lumen 1340, and at least one outer hole 1348a-1348f or porous segment from the side lumen 1340 to the exterior of the catheter is also provided. An elution hole occluder 1350 capable of resisting flow through the inner hole 1344, outer hole 1348 or both.

Medicament from the infusion lumen 1346 is capable of flowing through the inner holes 1344a-1344d, intersecting the side lumen 1340, and passing through the outer holes 1348a-1348f to exit from the catheter 1342 when the occluder 1350 is in a first, open position or has been withdrawn from the catheter. The inner holes 1344a-1344d and outer holes 1340a-1340f need not be aligned, and the number of inner 1344 and outer holes 1348 need not be equal. Inner hole 1344a and outer hole 1348a depict aligned holes whiles inner hole 1344d and 1348f depict non-aligned holes.

Any inner hole 1344 and outer hole 1348 capable of providing flow out of the catheter 1342 defines an elution hole or pathway. Any inner hole 1344 or outer hole 1348 may define more than one elution hole or pathway. For example, inner hole 1344c is capable of flow to outer holes 1348c-1348e. The cross-sectional areas of the inner holes and outer holes need not be equal and may vary within the same hole. In one embodiment, an inner hole 1344d has a greater diameter than outer hole 1348f. In one embodiment, a greater number of outer holes may be desired to create a more uniform elution pattern. In one embodiment, increased elution from outer holes that are closer to the inner holes can be reduced by decreasing the alignment between the inner holes and the outer holes to increase the tortuosity of the flow path and provide a more even distribution pattern from the outer holes.

The cross sectional shape of the elution holes can be circular, oval, square, triangular or any polygonal or closed shape. The cross sectional shape of the elution holes need not be uniform throughout the longitudinal length of the elution hole. In certain embodiments, the inner holes have a circular diameter of about 0.002″ and the outer holes have a rectangular shape, with a length of about 0.022″ as measured along the longitudinal axis of the catheter, and a width of about 0.007″. In one embodiment, a rectangular outer hole configuration where the width of the hole is about equal to the diameter of the occluder is used to provide better flow around some occluder configurations.

In one embodiment, the movable occluder 1350 is located generally along the length of the side lumen 1340, such as coaxially within the side lumen 1340. In one embodiment, the movable occluder 1350 comprises at least one narrow connector portion 1352 with a narrow diameter and at least one blocking portion 1354 which, in the illustrated embodiment, comprises an enlarged diameter or width that is capable of forming a seal with the side lumen. Movable occluders with a uniform diameter may also be used, but such occluders may exhibit increased resistance to sliding compared to occluders with variable diameters.

In sealing with the side lumen 1340, the enlarged portion 1354 may block an inner hole, an outer hole or both. FIG. 42A illustrates an occluder 1350 blocking inner hole 1344c and outer hole 1348f but not inner hole 1344d or outer holes 1348c to 1348e. By axially advancing the occluder 1350 either proximally or distally in the side lumen 1340, the relative position of the blocking portions 1354 and the corresponding elution holes may be changed and the effluent flow path may be selectively opened or closed. Not every hole needs to be blockable by the elution hole occluder. In one embodiment, the enlarged portions have longitudinal lengths that are at least as long as the diameter of the holes to resist medicament flow through the hole. The enlarged portions of the occluder may also be provided with longer lengths to decrease the precision with which the occluder is positioned within the side lumen in order to resist or occlude flow through the holes. The occluder and/or side lumen may also be provided with a lubricious coating or treatment to facilitate sliding of the occluder within the side lumen. Such coatings may include PTFE, paralene, or others known in the art. The occluder and/or side lumen may also be coated or treated to alter the sealing characteristic between the occluder and the side lumen.

In one embodiment, the side lumen has an internal diameter of about 0.025″ and the occluder comprises a valving wire with narrow portions having a primary diameter of 0.015″ and at least one enlarged portion with a diameter of about 0.022″ to about 0.024″ by about 0.200″ length. When the enlarged portion of the occluder is positioned next to an inner hole or outer hole, the elution hole or pathway defined by the inner hole and outer hole is “closed” and flow from the infusion lumen out of the catheter is blocked or resisted. When the enlarged portion of the valving wire is positioned away from a pair of inner and outer holes, the pair of holes is “open” and medicament is able to flow through the holes and out of the catheter.

In another embodiment, the occluder comprises a movable ribbon having narrow portions and wider portions that is capable of reversibly occluding the elution holes. Alternatively, the occluder may comprise a rotatable element, such as an elongate tubular body having side wall apertures aligned to permit or block fluid communication between the central lumen 1346 and one or more ports on the exterior wall of the catheter.

In one embodiment, the occluder is configured to generally open all of the elution holes or porous segment simultaneously. This allows the user to quickly initiate the fluid elution along the entire length of catheter, so that the dilution of the medicament by flowing blood is reduced. The risk of plugging or blocking the elution holes with clotted blood components may also be reduced by quickly opening generally all the elution holes.

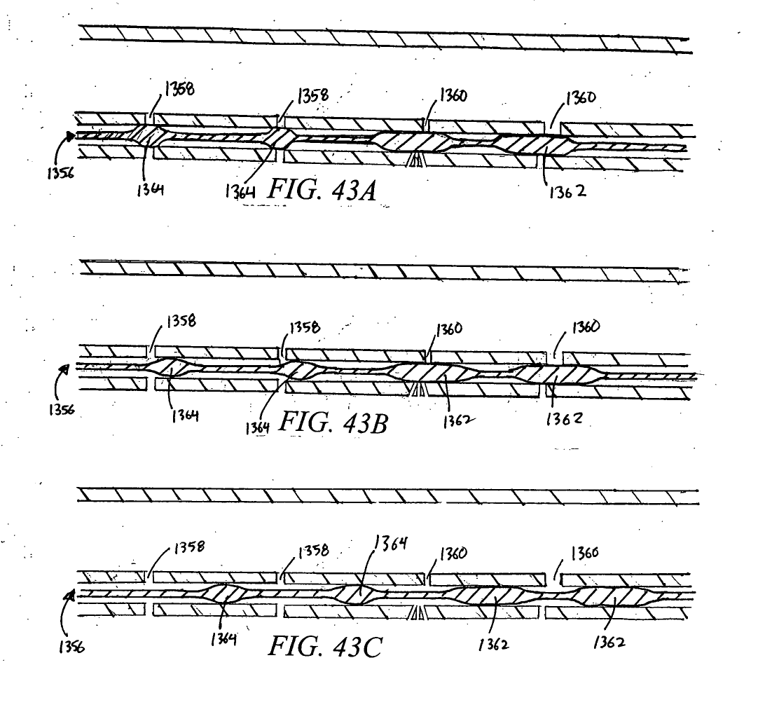

In certain embodiments of the invention, illustrated in FIGS. 43A to 43C, the length and number of the narrow portions and enlarged portions of the occluder are configured or arranged such that the occluder 1356 is capable of opening individual or a first group of the elution holes 1358 while a second group of elution holes 1360 remain closed. By providing the ability to open a limited number of elution holes while maintaining closure of other elution holes, the user can control the location of the effective elution zone and further customize the treatment procedure.

In one embodiment, the first position-of the occluder 1356, depicted in FIG. 43A, keeps all elution holes 1358, 1360 closed. In the second position illustrated in FIG. 43B, the increased length of the enlarged portions 1362 allows the occluder to keep holes 1360 in a first zone closed while the shorter length of enlarged portions 1364 allow the opening of holes 1358 in a second zone. In the third occluder position in FIG. 43C, all the holes 1358, 1360 in both the first and second zones are open. The spacing of the elution holes on the catheter may affect the additional number of occlusion patterns available.

In certain embodiments, the elution holes can be opened sequentially along the length of the delivery zone to provide and then closed, a moving elution zone without repositioning the catheter, or to allow a single catheter length to be used for treating patients requiring different delivery zone lengths. One example of the latter configuration comprises a catheter having a 44 cm delivery zone that is only partially inserted into a patient's leg because only a 24 cm delivery zone was required. The catheter will not leak sclerosant from the proximal 20 cm that lies external to the patient where the occluder is configured and positioned to only open the elution holes in the distal 24 cm of the catheter. In another embodiment, the occluder is configured so that the elution holes are opened in groups rather than individually, by either arranging the elution holes circumferentially in the same longitudinal region of the catheter, or by provide the enlarged portions of the occluder with sufficient length or particular spacing to simultaneously block multiple holes.

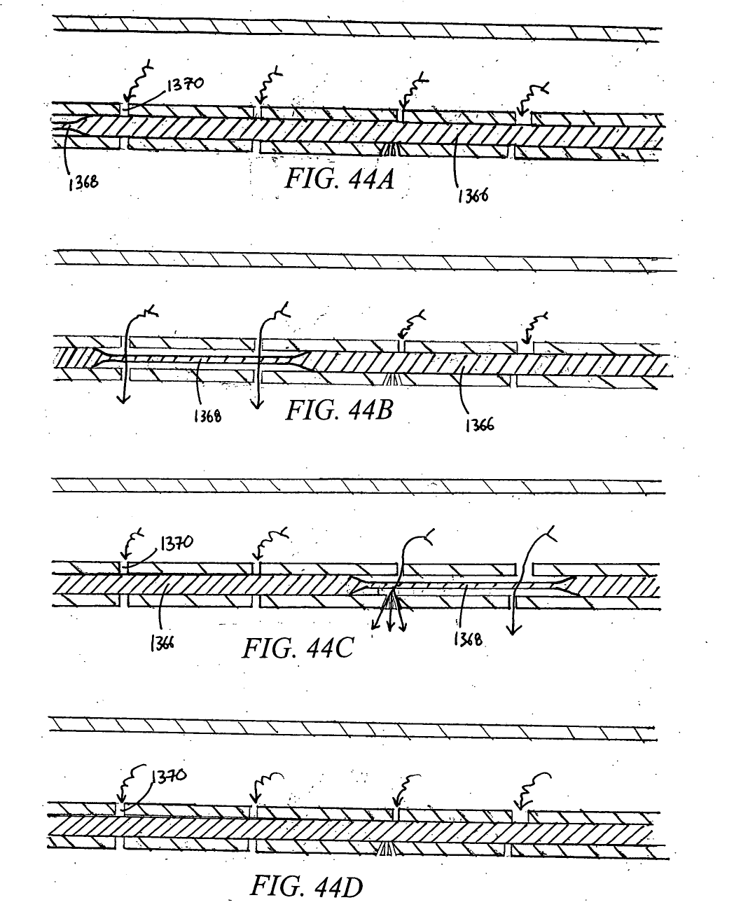

FIGS. 44A to 44D depict one embodiment, where the occluder is further configured to open an elution hole or group of holes and then close the elution holes prior to, during or after opening another group of elution holes. The occluder 1366 comprises a narrow segment 1368 that allows medicament flow through the elution holes 1370 adjacent to it. In one embodiment, the narrow segment 1368 is movable along the treatment length of the catheter to open the elution holes, two at a time. This particular embodiment may require a longer catheter length that extends beyond the occlusion balloon of the catheter to accommodate the distal end of the occluder. One skilled in the art will understand that the occluder may be configured to provide any of a variety of opening and closing patterns in the catheter by altering the length, position and number of narrow and enlarged portions on the occluder.

In one embodiment, an infusion catheter with an occluder capable of sequentially opening the elution holes may also be advantageous when infusing foam-based medicaments, including but not limited to sodium tetradecyl sulfate. The inventors have found that when elution holes with cross-section areas comprising a significant fraction of the infusion lumen cross-sectional area are used, it is common for liquid and foam-based medicaments to preferentially elute from the first hole that the foam encounters as it enters the catheter. In simple catheter constructions, this is typically the most proximal elution hole. Foam is typically disposed to elution in this manner because of its compressibility. During elution, the pressure of injection causes the foam to be compressed until it encounters an opening in the catheter, where it expands into the lower-pressure environment outside the catheter. To compensate for the increased elution of medicament at the proximal end of the catheter treatment zone, a catheter with a sequentially opening elution hole controller may be used. In one embodiment, to provide infusion of medicament along the entire length of the treatment zone, the most distal elution holes or elution zones are opened first, so that the medicament will elute from these distal areas. The adjacent proximal elution holes and/or elution zones are then sequentially opened to allow elution in a more proximal fashion. By using a sequentially opening catheter, a medicament that elutes primarily from the first-encountered elution hole may be dispensed evenly across the entire length of the catheter treatment zone. In one embodiment, elution control may be accomplished by proximally retracting a valving wire, but other control structures can also be used.

It may be advantageous for the catheter user to be able to elute a bolus of medicament at a specific location in the body, in addition to the even elution across the treatment zone of the catheter. Bolus treatment may be accomplished with a catheter comprising two elution systems: a) an “even-elution” system as previously described using a series of elution holes or pores which simultaneously or sequentially elute over a prescribed portion of the infusion catheter, and b) one or a series of sequentially-openable larger openings that will elute medicament (either foam or liquid) at a bolus delivery zone. Before, during or after performing an even elution, the operator may use the second system of larger holes to deliver a single or multiple boluses to specific areas in the blood vessel.

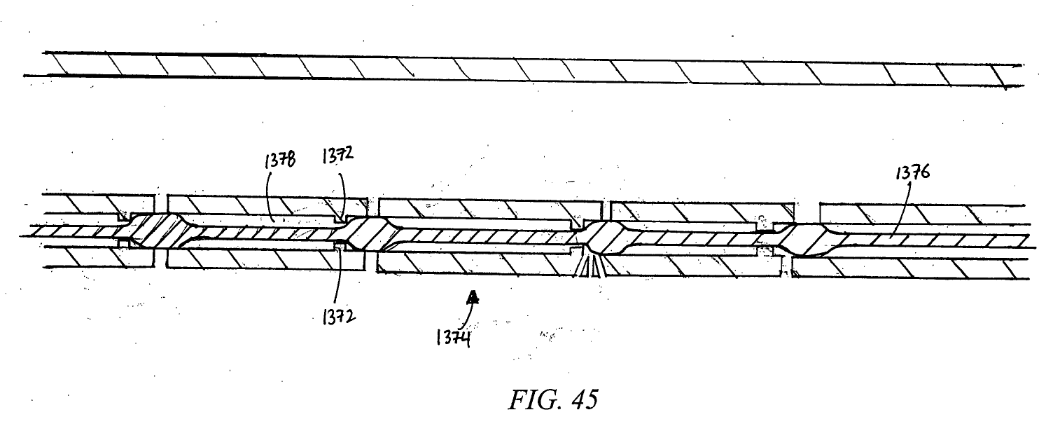

FIG. 45 shows one embodiment comprising one or more stops 1372 and/or detents in the infusion catheter 1374 to facilitate alignment of the valving wire 1376 within the side lumen 1378. The stops may restrict the sliding range of the wire 1376 and can prevent accidental removal of the wire 1376 from the side lumen 1378. The stops 1372 and/or detents may be located within the side lumen 1378 and/or in the proximal portion of the catheter 1374 at or about the infusion ports. Alternatively, the stop may be provided within or in the vicinity of a proximal manifold on the catheter to simplify manufacturing as will be appreciated by those of skill in the art. In one embodiment of the invention, the infusion catheter is supplied with a set of different valving wires that are insertable into the side lumen before or during the procedure, to allow further adjustment to the elution pattern of the catheter.

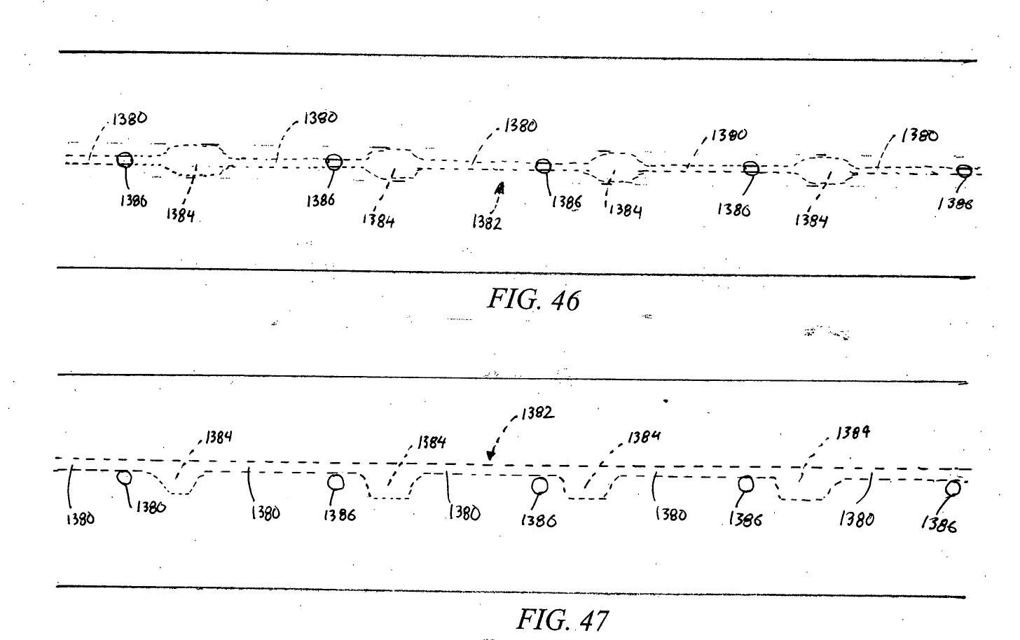

FIG. 46 illustrates one embodiment of the invention, where the narrow portions 1380 of the occluder 1382 are generally aligned with the enlarged portions 1384 along the same longitudinal axis such that when an elution hole is open, fluid from the inner hole must pass around at least a portion of the occluder with the narrow diameter to flow into the outer holes 1386. In another embodiment, depicted in FIG. 47, the primary portions 1380 of the occluder 1382 are joined eccentrically with the enlarged portions 1384, so that the primary portions 1380 offer less resistance to flow through the outer elution holes 1386.

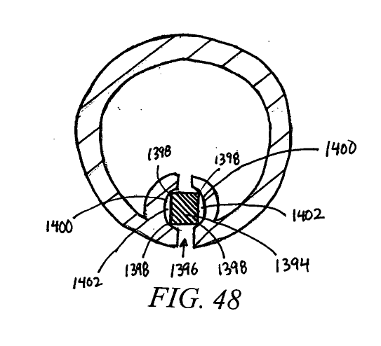

In one embodiment, shows in FIG. 48, the cross sectional shape of the occluder 1394 does not match the shape of the side lumen 1396. In one embodiment, by providing an occluder 1394 with a non-circular or oval cross-sectional shape, surface friction between the occluder 1394 and the side lumen 1396 may be reduced. In one embodiment, an occluder 1394 with a polygonal cross section is provided, where the edges 1398 of each polygon face are capable of providing sealing contact with the side lumen wall 1400, but the overall reduced friction allows the user to quickly move or remove the occluder 1394. In the illustrated embodiment, a four-cornered (square) wire 1394 is used in a circular side lumen 1396 as an occluder. At least one sealing line at one of the wire corners 1398 is capable of forming sealing contact with the side lumen 1396. Although potential leakage paths 1402 may exist along the longitudinal length of side lumen 1396 because of the lack of complete surface-to-surface contact between the wire and the side lumen walls, the length of the leakage paths are likely to be of sufficient length so as to substantially reduce or prevent elution of medicament or intrusion of blood components at the side lumen 1396.

In one example, an infusion catheter comprising a side lumen and an array of ten elution holes, with one hole per centimeter over a nine centimeter length, is provided. The side lumen contains a single square wire of at least about 9 cm length. In one embodiment, a smaller-diameter pull wire is engaged the proximal end of the square wire, to allow manipulation of the square wire from the proximal end of the catheter. In an alternate embodiment, to simplify manufacture of the square wire occluder, a square wire with a length at least sufficient to extend from through the proximal end of the catheter to the distal end of the catheter treatment segment is used as an occluder. In one embodiment, short segments of the wire may have cross-sections closer to or matching that of the side lumen to limit the extent of lengthwise leakage, without significantly increasing the net sliding friction of moving or withdrawing the wire from the catheter.

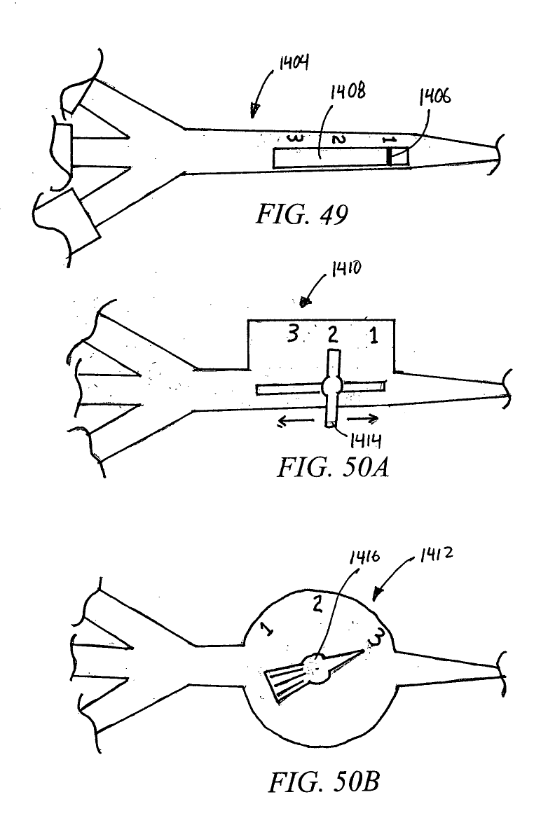

FIGS. 49, 50A and 50B depict optional indicators on the catheter to provide information regarding the position of the occluder, the open/close status of the elution holes, or both. In one embodiment, shown in FIG. 49, the indicator 1404 is a marker such as a colored bank carried by the occluder 1406 another that is capable of moving within a window 1408. In another embodiment, schematically illustrated in FIG. 50B the indicator comprises a dial turned relative to an index mark by a rack-and-pinion or friction drive. One skilled in the art will understand that other mechanisms for indicating the position of the occluder or status of the elution holes may be used. In one embodiment of the invention, shown in FIGS. 50A and 50B, the indicator 1410, 1412 is incorporated or combined with an occluder actuator 1414, 1416 for manipulating the position of the occluder. The occluder actuator may comprise a slider 1414, lever, or turning knob 1416 attached to the occluder. The occluder actuator may also comprise a servo motor that is electronically controllable by the user. One skilled in the art will understand that other mechanisms for moving the occluder may also be used.

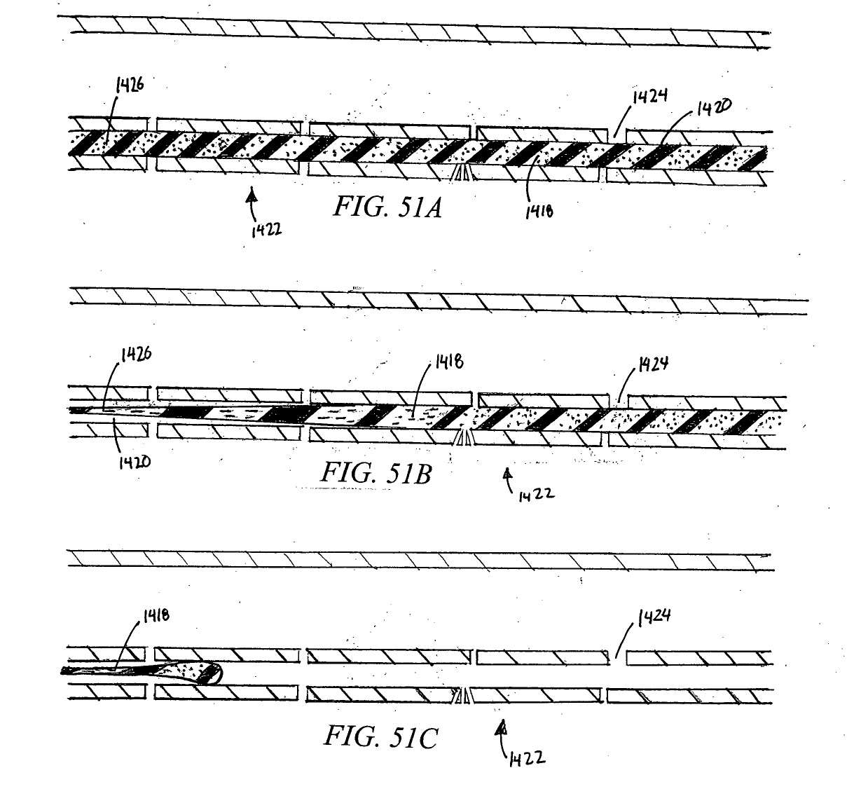

FIGS. 51A to 51C depict one embodiment of the invention, the movable occluder comprises an elastomeric cord 1418 within the side lumen 1420 of the catheter 1422. Such a cord may comprise latex, silicone rubber, natural rubber, neoprene and other chloroprene variants, polyurethane, ethylene-propylene, polyvinyl chloride, polyamide, polyamide elastomer, copolymer of ethylene and vinyl acetate, polyethylene, polyimide, polyethylene terephthalate, fluorine resin, polyisobutylenes or other thermoset elastomers, polyisoprene, or any of a variety of resilient materials known in the art. The cord may have a cross-sectional shape that is square, rectangular, oval, circular, polygonal or any of a variety of other shapes that are capable of forming a seal with the side lumen. The cord may be solid, hollow or have a core comprising the same or different material. In one embodiment, at least one portion or segment of the elastomeric cord has a native diameter that is larger than the inside diameter of the side lumen 1420, to provide enhanced occlusion of the elution holes 1424. As shown in FIG. 51B and 51C, by pulling on the proximal end 1426 of the cord 1418 and causing longitudinal lengthening, the cord 1418 is capable of deforming and reducing its cross-sectional area, as shown in the proximal end 1426 in FIGS. 51B. This reduction in diameter allows the cord to be removed from the side lumen and opens the elution holes 1424.

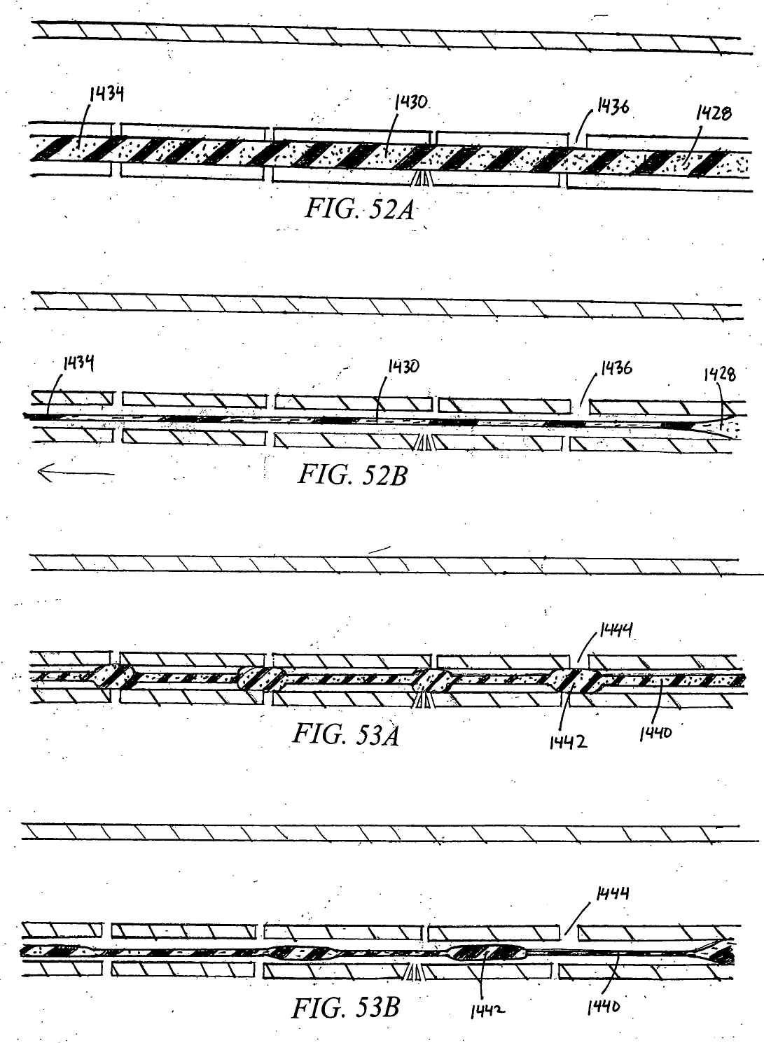

In another embodiment shown in FIGS. 52A and 52B, the distal end 1428 of the cord 1430 is anchored in the side lumen 1432 so that the cord 1430 resists removal from the side lumen 1432 when a pulling force is applied to its proximal end 1434, but is capable of decreasing in diameter or cross sectional area sufficiently to allow flow through the elution holes 1436. Anchoring may be accomplished using any of a variety of techniques, such as adhesives, solvent or thermal bonding, mechanical interfit, cross pins or others known in the art. In one embodiment, upon cessation of the proximal pulling force, the cord 1430 is generally able to revert back to its previous length and diameter and reversibly re-close the elution holes. In another embodiment, upon pulling the cord 1430, the cord plastically deforms and some or all of the elution holes 1436 remain at least partially open after cessation of the pulling force. In one embodiment, illustrated in FIG. 53, the elastomeric cord 1438 comprises narrow segments 1440 and enlarged segments 1442 or increasing the sealing characteristics of the cord 1438 at the elution holes 1444 and/or to reduce the tensile force needed to move or remove the cord 1438 in the side lumen 1446. In one embodiment, the elastomeric cord and/or side lumen is coated or treated to alter the friction between the cord and lumen.

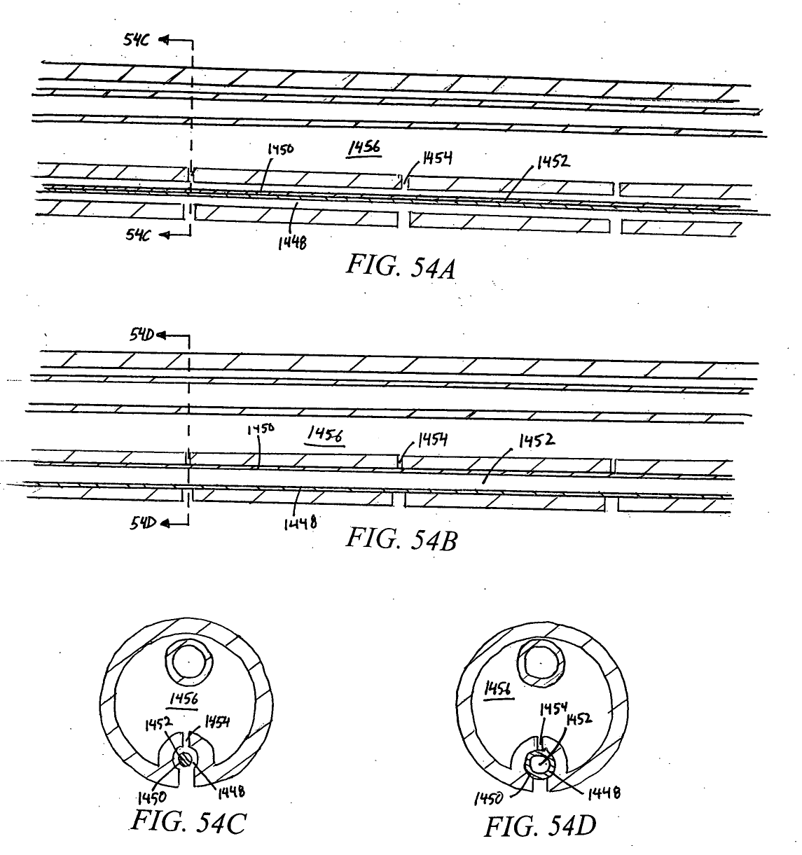

FIGS. 54A to 54D depict another embodiment of the invention, in which a hollow flow regulating tube 1450, having a central lumen 1452 is positioned within the side lumen 1448. The tube 1450 has an open proximal end and a closed distal end. The proximal end may be provided with a releasable connector such as a Luer fitting for connection to a source of inflation media. Alternatively, the central lumen may be in direct communication with a variable volume chamber in the proximal manifold or hand piece for the catheter.

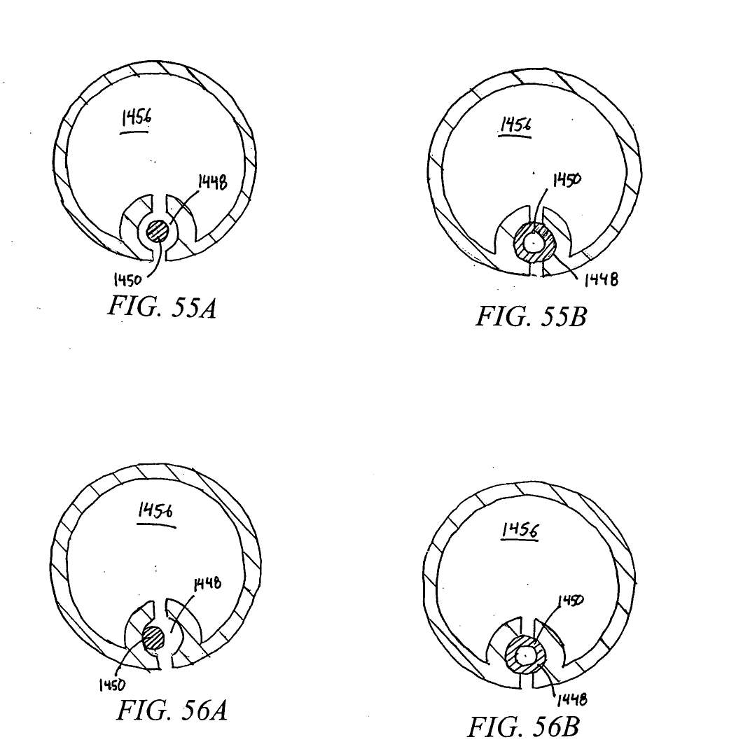

The outside diameter of the flow regulating tube 1450 is moveable from a first, reduced diameter to a second enlarged diameter upon introduction of inflation media into the central lumen 1452. The outside diameter of the tube 1450 in the first, relaxed configuration is less than the inside diameter of the lumen within which it resides, such as side lumen 1448. In this configuration, a medicament or other agent in the infusion lumen 1456 is capable of flowing past or around the hollow tube 1450 to exit out of the elution hole 1454. See FIG. 55A. Introduction of inflation media into central lumen 1452 causes an enlargement of the outside diameter of the tube 1450 such that it occludes the flow path between the infusion lumen 1456 and the exterior of the catheter body. See FIG. 55B.

The flow regulating tube 1450 thus provides a movable wall which may be advanced between a first orientation in which flow is permitted to occur and a second orientation in which flow is inhibited. Introduction of intermediate pressures into the central lumen 1452 may be utilized to regulate flow at intermediate flow rates, or permit flow only to occur when the driving pressure within the infusion lumen 1456 exceeds a predetermined threshold.

Although the flow regulating tube 1450 is described as located within the side lumen 1448, valves or flow regulators which are responsive to changes in pressure may be incorporated into the catheter of the present invention in any of a variety of ways. For example, the inflatable tube 1450 may be positioned within the inflation lumen 1456, and the side lumen 1448 may be eliminated or utilized for another purpose. The inflatable tube 1450 may be configured to have an axial length less than the length of the infusion zone, such that, for example, it occludes only a relatively proximal portion of the catheter body. In one implementation, the flow regulating tube 1450 has an axial length of no greater than 2 or 3 or 4 times the inflated diameter, such that it operates as an inflatable valve positioned in-between the proximal most elution hole and the source of infusion media. In general, however, it appears desirable for the axial length of the flow regulating tube 1450 to be at least as long as the infusion zone, such that in the inflated configuration, the flow regulating tube 1450 physically occludes each elution hole 1454.

The escape of material from the infusion lumen 1456 through each elution hole 1454 may be accomplished by providing an inflatable tube 1450 at any point between that elution hole 1454 and the source of infusion media. However, it also appears desirable to block each elution hole 1454 to prevent blood or other body fluid from entering the catheter in a retrograde flow direction, prior to the time that the sclerosant or other infusion media is infused from the catheter into the patient. Thus, in accordance with the present invention, there is provided a method and related device for introducing a catheter into a patient, the catheter having a plurality of elution holes 1454, and preventing the introduction of body fluid into the catheter through the elution holes. The introduction of body fluid into the catheter is inhibited by the positioning of a movable wall across the elution hole. The moveable wall is moveable between a first position in which it occludes the elution hole 1454, and a second position in which the infusion lumen 1456 is in communication with the exterior of the catheter through the elution hole 1454. In the illustrated embodiment, the moveable wall is the surface of an inflatable tube, although other structures for moving a wall between a first position and a second position may also be utilized.

Although the present embodiment has been described primarily in terms of a hollow flow regulating tube 1450 having a reduced outside diameter in its relaxed configuration, the device may alternatively be constructed such that the hollow flow regulating tube 1450 resides in an enlarged cross sectional diameter in it relaxed configuration. This configuration would provide a “normally closed” valve system, in which the outside diameter of the flow regulating tube 1450 would normally occlude the elution hole 1454. In this construction, drawing a negative pressure on the central lumen 1452 could be utilized to reduce the cross sectional area of the flow regulating tube 1450, thereby placing the elution hole 1454 into communication with the infusion lumen 1456.

The tube 1450 may comprise any of a variety of materials that may be expanded under pressure, such as latex, silicone rubber, natural rubber, neoprene and other chloroprene variants, polyurethane, ethylene-propylene, polyvinyl chloride, polyamide, polyamide elastomer, copolymer of ethylene and vinyl acetate, polyethylene, polyimide, polyethylene terephthalate, fluorocarbon resin, polyisobutylenes and other thermoset elastomers, polyisoprene, or any of a variety of materials known in the art that is capable of radial expansion when fluid in the hollow portion 1452 of the tube 1450 is pressurized.

In one embodiment, depicted in FIGS. 55A and 55B, the elastomeric tube 1450 is positioned concentrically within, or is allowed to “float” within the side lumen 1448 in both the inflated and deflated states. In another embodiment, shown in FIGS. 56A and 56B, the elastomeric tube 1450 in the deflated state is positioned eccentrically in the side lumen 1448 using a sealant, adhesive, thermal welding or other bonding technique known in the art. FIG. 56B shows that when tube 1450 is fully expanded, it can assume a more concentric position in the side lumen 1448. In one embodiment, an eccentric position may provide a larger or more predictable effective flow path past the elastomeric tube 1450 compared to a concentrically positioned or free floating tube 1450.

The ratio of the first, reduced diameter of the flow regulating tube 1450 to the inside diameter of the lumen within which it resides can be varied widely, depending upon the desired performance characteristics, taking into account the viscosity and desired flow rate of the infused media. In general, the deflated diameter of the tube 1450 will be no greater than about 75% of the inside diameter of the side lumen 1448. In certain constructions, the deflated outside diameter of the flow regulating tube will be no more than about 65%, and, in certain implementations, no greater than about 60% of the inside diameter of the lumen within which it is contained.

In certain constructions, the hollow elastomeric tube 1450 has a deflated outside diameter ranging from about 0.008″ to about 0.100″. In certain embodiments, the tube 1450 has a deflated outside diameter ranging from about 0.010″ to about 0.050″. The elastomeric tube has a deflated internal diameter generally within the range of from about 0.003″ to about 0.080″. In a preferred embodiment, the elastomeric tube has an outer diameter of about 0.015″ and an inner diameter of about 0.006″, for use in a lumen having an inside diameter of about 0.025″.

The inflation pressure sufficient to occlude the elution holes may range from about 10 pounds per square inch (psi) to about 1000 psi. In certain embodiments, the occlusion pressure is about 50 psi to about 500 psi. In another embodiment, the occlusion pressure is about 100 psi to about 600 psi. In one embodiment, where the occluder comprises an elastomeric tube with an outer diameter of about 0.015″ and an inner diameter of about 0.006″ in a 0.025″ side lumen, the tube has an occlusion pressure at about 100 psi to about 200 psi.

The tube diameter, wall thickness, wall compliance, and other tube characteristics may be varied along the length of the bladder tube. One skilled in the art may alter these characteristics to provide different occlusion characteristics across a pressure range. In one example, a bladder tube may be designed to sequentially deflate from distal to proximal over a pressure range from 200 psi to 100 psi. Distal to proximal deflation may be accomplished, for example, by providing a first wall thickness for the elastomeric tube 1450 in the proximal end and a second, greater wall thickness for the elastomeric tube 1450 near the distal end. Wall thickness may be graduated continuously from the proximal end to the distal end. Alternatively, deflation may be accomplished initially at the proximal end by providing the greater wall thickness at the proximal end. As will be apparent to those of skill in the art in view of the disclosure herein, the inflation characteristics of the foregoing constructions will be the reverse of the deflation characteristics, such that portions of the flow regulating tube with a relatively lesser wall thickness will inflate at a lower pressure than portions of the flow regulating tube with a greater wall thickness. The sequential expansion during inflation may occur smoothly across the length of the flow regulating tube, or in a segmented fashion. In another example, the bladder tube may comprise dimples in the bladder tube that evert and occlude elution holes at a particular pressure threshold.

In one embodiment of the invention utilizing an inflatable flow regulator form of occluder, the occluder comprises an inflatable tube in a catheter with outer hole diameters of about 150 microns or greater and inner holes diameters of about 200 microns or less. In another embodiment, the catheter comprises outer hole diameters of about 400 microns or less and inner hole diameters of about 5 thousandths of an inch (200 microns) or more. In one embodiment, the outer holes have diameters of about 200 microns or more and inner holes of about 20 microns to about 250 microns. In another embodiment, the outer holes have diameters of about 20 microns to about 250 microns and the inner holes have diameters of about 200 microns or more. In one embodiment, at least either the outer holes or inner holes have a diameter of about 8 microns to about 175 microns. In a preferred embodiment, the catheter comprises outer holes with diameters of about 300 microns or greater and inner holes with diameters of about 50 microns to about 175 microns. The inner holes may have the same, a smaller, or a larger diameter than the corresponding outer hole.

The elastomeric tube may be pressurized with a pressure controller comprising variable volume container such as a syringe. The syringe may have a capacity of about 0.25 cc to about 25 cc, and may be is attachable such as by a Luer connector to the proximal end of the inflatable tube. In certain embodiments, the syringe has a capacity of about 1 cc to about 5 cc. In a preferred embodiment, the syringe has a capacity of about 1 cc to about 2 cc.

The plunger of the syringe may be controlled directly by the operator or through a lever or knob with detent. In another embodiment, the pressure controller comprises an electronically controlled pump and pressure release valve. One skilled in the art will understand that any of a variety of pressure controllers may be used. In one embodiment, the syringe or catheter further comprises a stopcock for maintaining pressure in the elastomeric tube without further effort by the user. In another embodiment, the plunger or tube controller further comprises a latch for maintaining the position of the plunger. In a preferred embodiment, the tube controller provides a two-position control of the tube where the tube is either inflated or deflated. In another embodiment, the pressure controller is capable of providing multiple degrees of tube pressurization. A controller providing multiple degrees of tube pressurization may be useful to provide variable flow patterns or varying degrees of flow through the elution holes to further control the flow rate of medicament out of the catheter.

In one embodiment of the invention, the hollow elastomeric tube is pressurized with a gaseous medium. In one embodiment, the tube is pressurized with a liquid medium. A liquid medium may be preferred to decrease the risk of an air embolus in the venous system that may travel to the lungs or other sites and block tissue perfusion.

In one embodiment of the invention, the elastomeric or bladder tube comprises silicone or other porous material that is sufficiently permeable so that any trapped gas in the tube can be expelled by inflating the tube with a liquid to at least about 100 psi. Under such a pressure, the gases diffuse out through the permeable tube and/or into the liquid medium. In another embodiment, the bladder tube comprises a material such as neoprene that is generally permeable to gas but not to a liquid, such that when pressurized with a liquid, gases are allowed to escape through the pores of the material but liquid is retained. In another embodiment, any trapped gas in the tube is expelled by inflating the tube with a liquid to at least about 40 psi. In another embodiment, any trapped gas in the tube is expelled by inflating the tube with a liquid to at least about 200 psi.

In one embodiment, the catheter and/or syringe further comprises an indicator of elution hole occlusion by the bladder tube, or pressure in the bladder tube. In one embodiment, the indicator comprises markings on the pressure controller, such as the syringe or syringe plunger. In one embodiment, a pressure indicator independent of the pressure controller or pressure actuator is provided in the catheter. An independent pressure indicator may be advantageous over other mechanisms of pressure status in situations where leakage or failure of the bladder tube has occurred. For example, in a catheter where the bladder tube has ruptured, a plunger position marker on a syringe will indicate that a leaking bladder tube is fully pressurized, while an independent pressure indicator may accurately show that the bladder tube is unpressurized even though the plunger is fully depressed. In one embodiment, a poppet-type pressure indicator is attached to the catheter to indicate pressurization of the bladder tube. In another embodiment, a MEMS type pressure sensor is provided on the catheter to indicate the pressure status of the bladder tube. One skilled in the art will understand that any of a variety of pressure detection mechanisms may be used for a pressure indicator for the bladder tube.

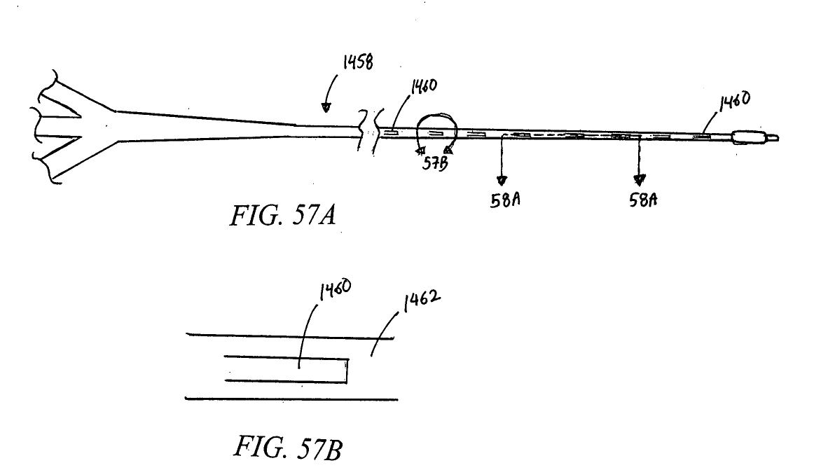

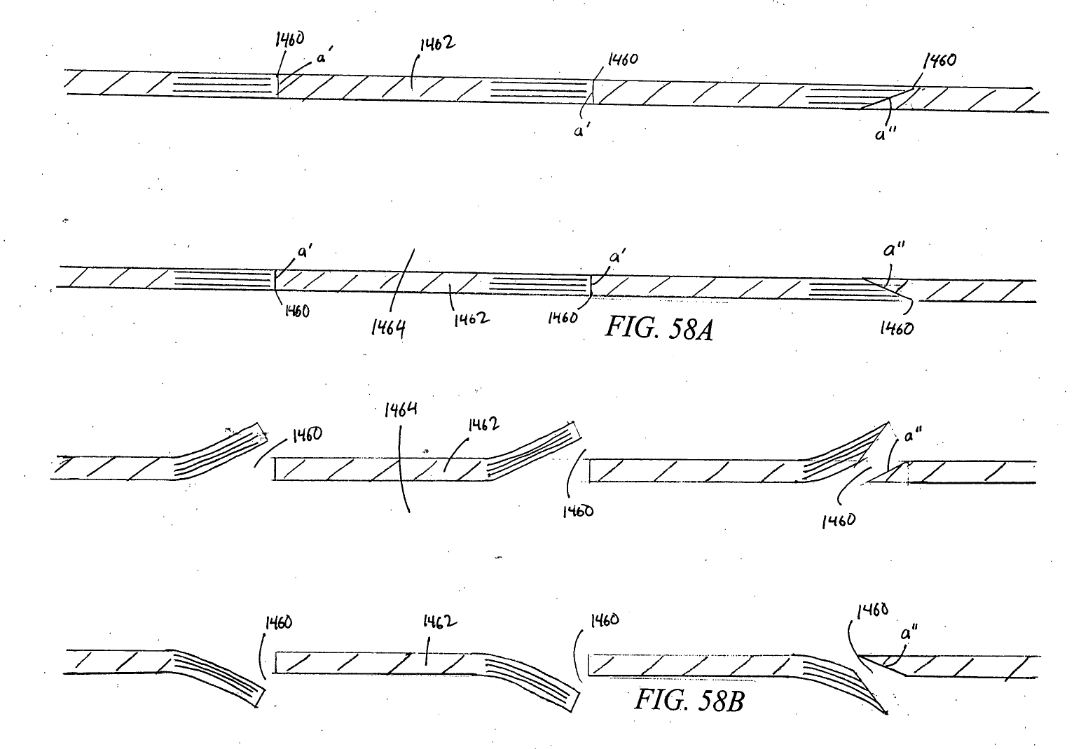

In accordance with another embodiment of the invention, the elution holes of the catheter 1458 comprise a plurality of slits in the outer catheter wall 1462 through which medicament is able to pass. FIGS. 57A and 58B show embodiments where the slits are provided in a “u” configuration, to produce an aperture with a hinged cover. The cover is normally closed and capable of resisting entry of blood components into the aperture to prevent clogging. When sufficient pressure is placed on the medicament within the infusion lumen 1464 of the catheter 1458, the cover 1460 will deform and open to allow the medicament to exit the catheter 1458.

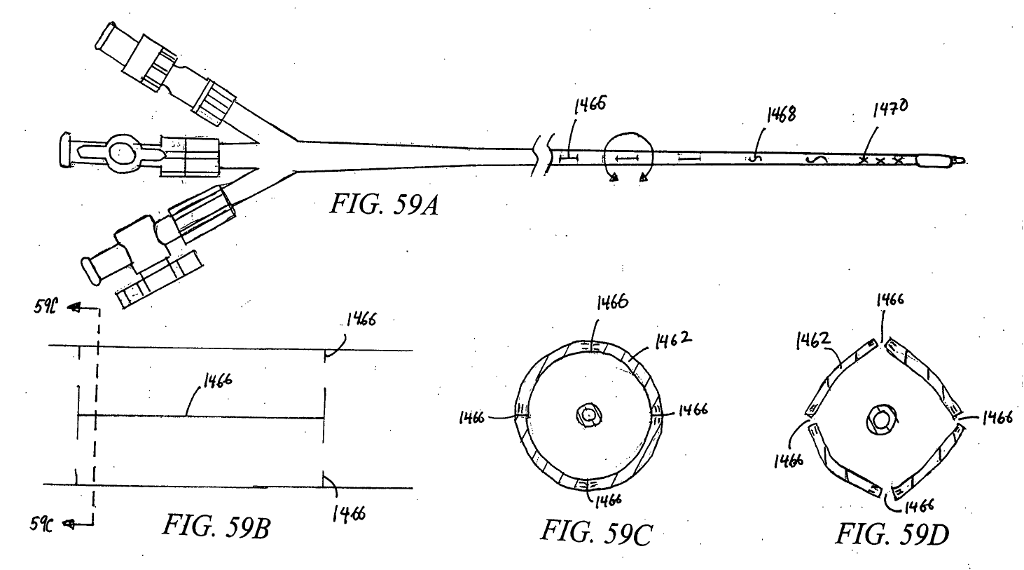

In one embodiment, the angle a′ of the slit between the external surface of the catheter to the inner surface of the catheter to form the cover 1460 is at a 90 degree angle to the surface of the catheter. In another embodiment, the slit angle a″ may be anywhere from about 1 degree to about 179 degrees to the catheter surface. FIGS. 59A to 59D shows that the slits may comprise any of a variety of configurations, including but not limited to simple lines, H-shapes 1466, S-shapes 1468, X-shapes 1470, star-shapes or U-shapes. One skilled in the art will understand that any of a variety of slit shapes may be used. Each slit on the catheter need not have the same shape, size or angular orientation. By changing the size or shape of the slits and/or by selecting the catheter wall thickness and material at the slit location, among other factors, one skilled in the art may configure the slit to open at a desired pressure or range of pressures.

One advantage of slit-based elution holes is the higher pressure required to open the slit valves. The higher opening pressure reduces the influence that the infusion pressure may have on the elution or flow pattern along the length of the catheter, due to the pressure drop along the length of the catheter. For example, in a catheter where there is a viscous pressure drop from the most proximal elution hole to the most distal elution hole of 20 psi and the slits open at a pressure of about 80 psi, if the pressure at the most proximal hole is 100 psi, the flow rate out of the most distal elution whole will be approximately 80/100ths or 80% of the flow rate out of the most proximal elution hole, because the pressure at the most distal hole will be about 80 psi. Where the catheter slits are configured to open at 100 psi (and making a simplifying assumption that flow is proportional to pressure once the slit is opened), if the pressure at the most proximal elution slit is 200 psi, the pressure at the most distal slit is 180 psi. The resulting flow from the most distal slit would be about 180/200ths or 90% of that at the most proximal slit. By altering the configuration of the slits, a catheter may be configured to provide an even elution pattern, or any other elution pattern, independent of the location of the slits along the catheter.

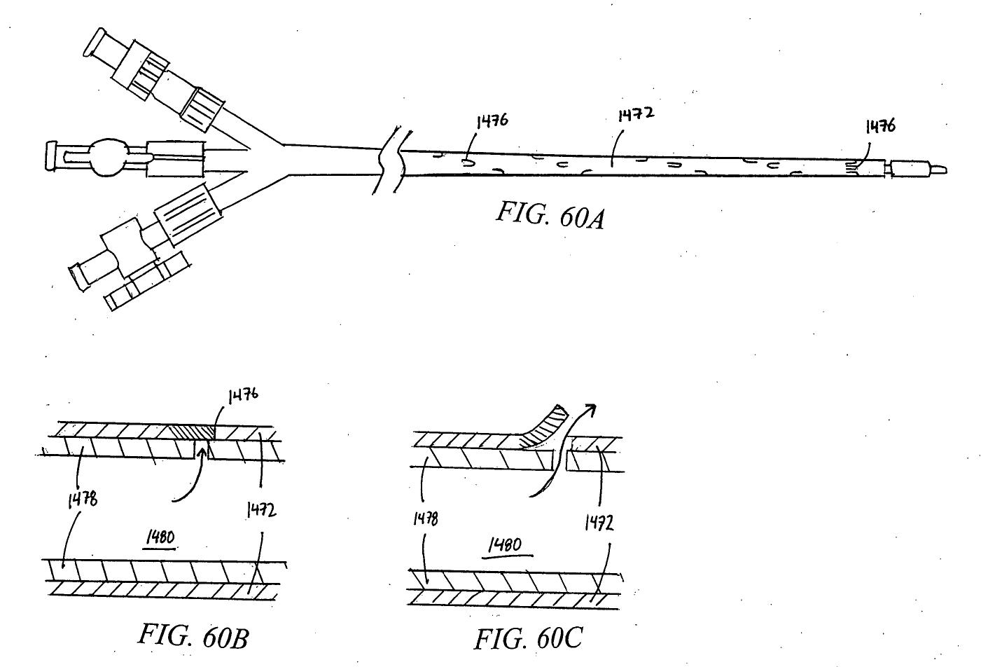

FIGS. 60A to 60C depict one embodiment of the invention with an elastic covering 1472 over the elution holes 1474 to prevent blood components from entering and clogging the holes. In one embodiment, the elastic covering comprises flaps or slits 1476 that form normally closed valves overlying the outer catheter wall 1478. When medicament in the infusion lumen 1480 is eluted from the catheter under pressure, the slit valves 1476 open to allow the fluid to egress, but close when the elution flow stops. In one embodiment, shown in FIGS. 60B and 60C, the slits 1476 in the elastic covering 1472 are positioned directly over the elution holes 1474 to provide a short path for the medicament to exit the catheter. In another embodiment, the slits in the elastic covering are not located directly over the elution holes so that the medicament takes a longer path from the elution hole to reach a slit. A longer path may be advantageous to further reduce blood ingress into the elution holes. In one embodiment, the number of slits does not match the number of elution holes on the catheter and allows for a distribution of the medicament that differs from that provided by the elution holes of the catheter. In one embodiment, the elastic covering is integral with the other portions of the catheter. In another embodiment, the elastic covering is attachable to the catheter just prior to insertion of the catheter into the patient. The user may be provided with a variety of elastic coverings each configured to provide a different elution pattern. The user can select and attach the desired elastic covering best suited to the anatomy of the patient.

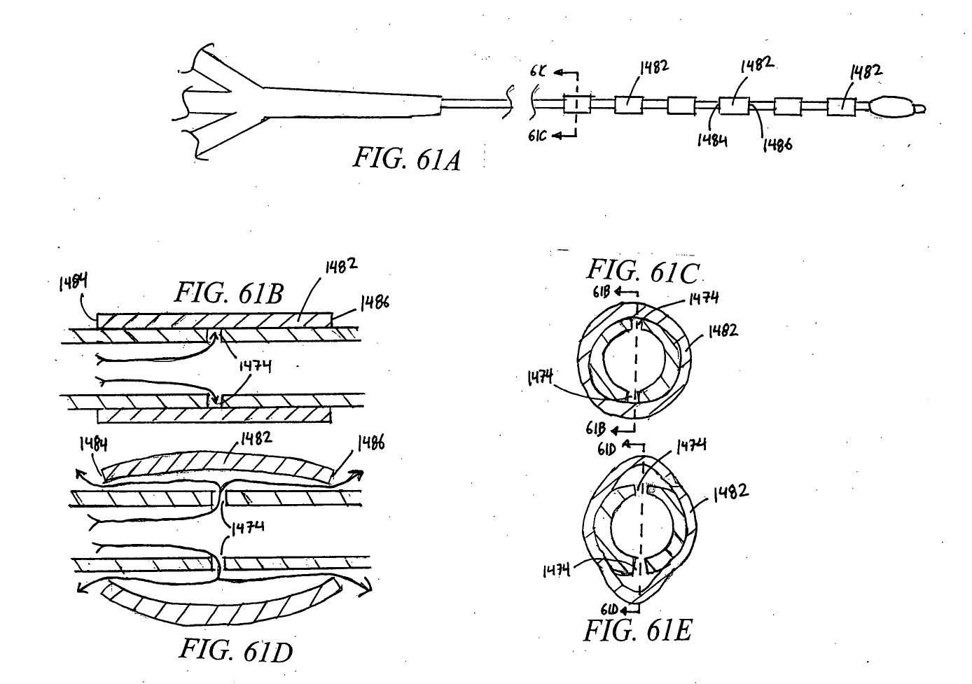

In one embodiment of the invention, as shown in FIG. 60A, a single contiguous elastic covering 1472 is located over the treatment portion of the catheter. In another embodiment, multiple short lengths of elastic covering, such as elastic rings, are used over the elution holes. FIGS. 61A to 61E shows still another embodiment of the invention, comprising multiple short lengths 1482 of elastic covering over the elution holes 1474, but where the elastic coverings lack slits so that the medicament flows out of the edges 1484, 1486 of the elastic coverings 1482. In FIGS. 61D and 61E, where multiple short circumferential bands 1482 of elastic coverings are engaged to the catheter, the medicament can flow out of the proximal 1484 and distal ends 1486 of each elastic band 1482.

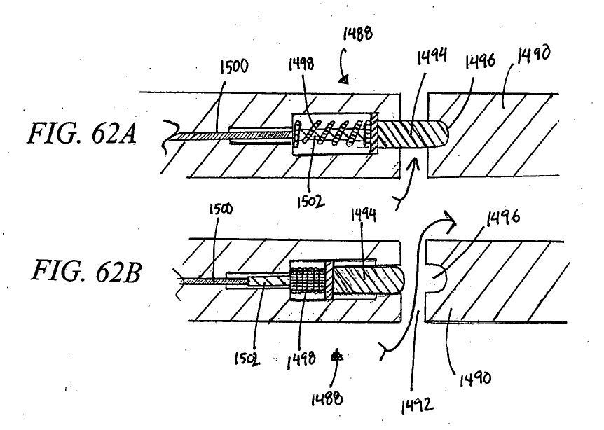

FIGS. 62A and 62B illustrate one embodiment of the invention comprising miniature gate-type valves 1488 incorporated into the catheter wall 1490 so that the flow through the elution holes 1492 can be individually changed or adjusted under active control by the clinician to achieve a variety of elution patterns and to maintain a closed configuration when elution is not taking place to prevent clogging from ingress of blood components into the elution holes 1492. In one embodiment such valves 1488 may be created using micro-machining techniques. In one embodiment, the valve head comprises a ball or pin with a diameter of about 0.002″ to about 0.080″. In a preferred embodiment, a 0.020″ diameter ball or pin 1494 may be positioned against a valve seat 1496 to close the elution hole 1492 with a small compression spring 1498 made from stainless steel wire. In one embodiment, the gate-type valve is contained within a machine or molded housing incorporating a valve seat 1496. The balls or pins 1494 may be made from tungsten carbide, stainless steel, glass or sapphire. In one embodiment, the springs 1498 may be made from 0.002″ wire wound to a 0.018″ outside diameter spring with a 0.02′ length. The valve is opened by exerting a pulling force on a control wire 1500 attached to the proximal end 1502 of the valve head 1494. The control wire extends proximally to a control such as a slider switch, trigger or rotatable know which may be carried by the proximal manifold. The spring will close the valve when insufficient pulling force is exerted. One skilled in the art will understand that a variety of gate-type valve configurations and sizes may be used to achieve the desired catheter characteristics.

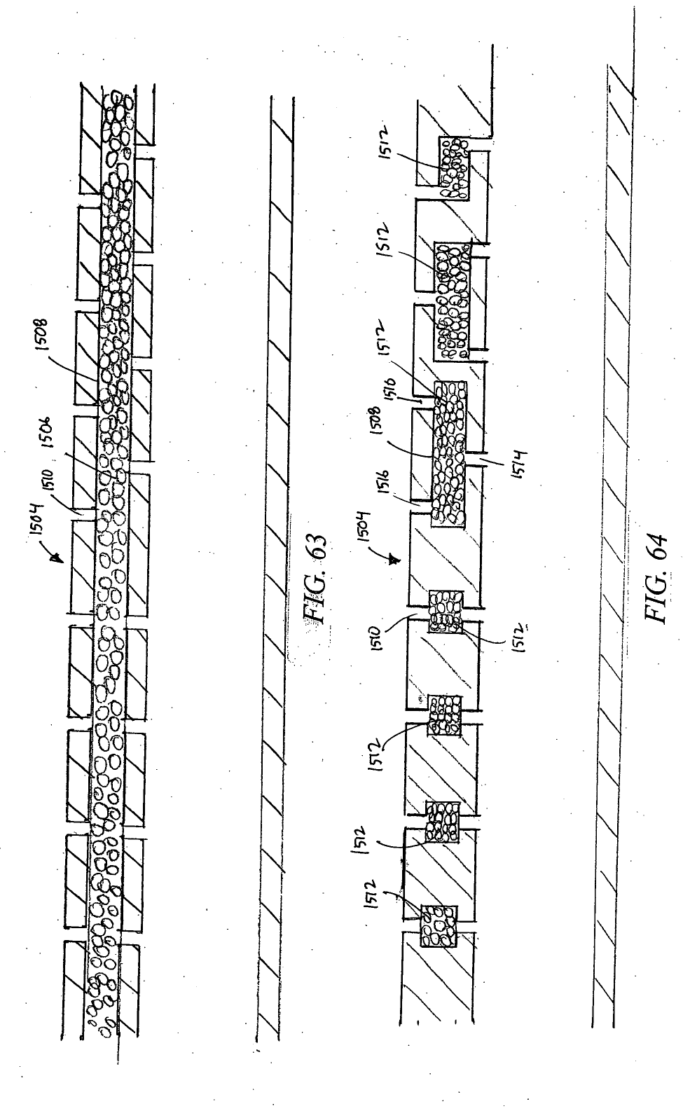

In one embodiment of the invention, shown in FIGS. 63 and 64, the elution holes 1510 of catheter 1504 are protected from clogging by blood components by a filter 1506 located within the side lumen 1508 of the catheter 1504. The filter comprises a permeable rod or string with a porosity of about 8 microns or less that is capable of excluding blood components. Such materials include but are not limited to Gore-tex® ePTFE, DuPont Tyvek® spun-bonded polyolefin or Millipore® microporous filter media, or any of a variety of porous organic or inorganic filter media known in the art. In one embodiment, a filter substrate with hydrophobic properties may be used to enhance exclusion of the aqueous blood components from the elution holes. In another embodiment, a filter substrate with hydrophilic properties may be used. Hydrophilic filters may be advantageous because they preserve foam-based medicaments as the foam passes through the filter, rather than break down the foam into fluid and gaseous components.

FIG. 63 depicts one embodiment of the invention, where a single filter substrate 1506 is provided generally along the entire length of the side lumen 1508. In another embodiment, multiple discreet filter units 1512 are provided for the elution holes 1510. The number of inner holes 1514 and outer holes 1516 served by a single filter unit 1512 need not be equal, as shown by the holes 1514, 1516 in FIG. 64. Discreet filter units may decrease the amount of lateral flow of treatment agent in the side lumen, thereby providing greater control of elution rate at any given catheter segment. One with skill in the art will understand that a catheter side lumen may be configured with both the filter and an elution hole controller.

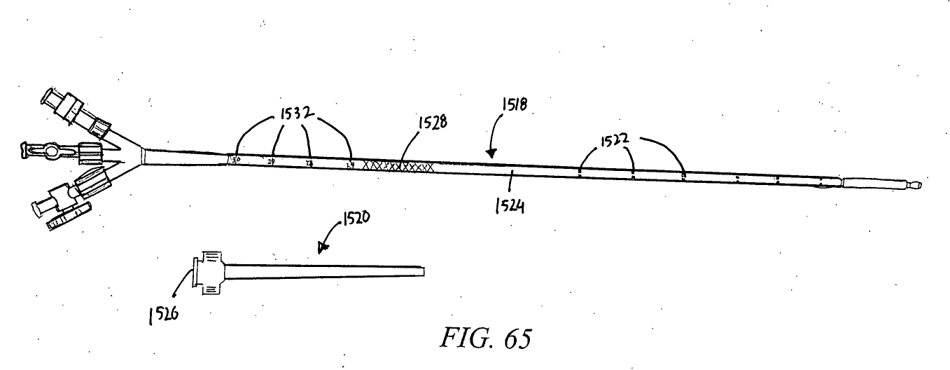

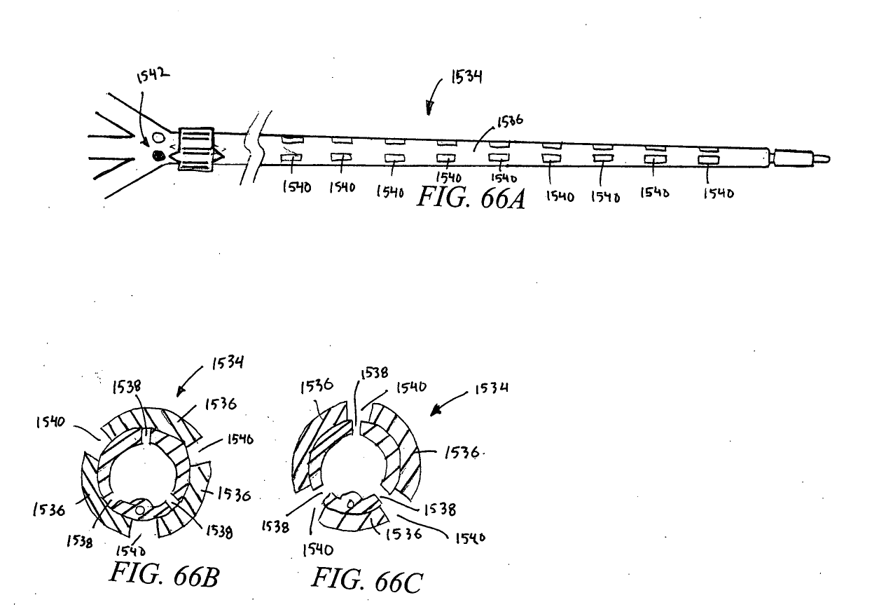

In one embodiment of the invention, shown in FIG. 65, one or more visualization markers are provided, such as on the exterior surface of the catheter 1518. Used in conjunction with the catheter sheath introducer 1520, the user is able to determine the location of the treatment zone relative to external fiducial markers on the body and whether any elution holes 1522 of a partially inserted catheter 1518 are being blocked by the catheter sheath introducer 1520. In one embodiment, the user is able to view the exposed markers located proximally on the catheter body 1524, relative to another landmark on the introducer 1520, such as the most proximal end 1526 of the introducer 1520. One marker region 1528 on the catheter body 1524 informs the user that the proximal elution holes of the catheter 1518 are within the introducer 1520. Interval markers 1532 convey to the user the distance from the introducer to some defined position on the catheter. This defined position may be the most proximal elution hole, the most distal elution hole, the blood vessel occluder position, or any of a variety of sites on the catheter. Knowledge of the catheter position relative to the introducer allows the user to properly position the infusion catheter to the patient's anatomy and to provide the desired elution pattern.

FIG. 66 depicts another embodiment of the invention, comprising a catheter 1534 with a rotatable control tube 1536 overlying the elution holes 1538 of the catheter. In one embodiment, the control tube 1536 has a plurality of windows 1540 arranged along the length of the tube 1536 and is rotatable to at least two positions, as indicated by proximal markers 1542. In a first position, shown in FIG. 66B, at least one elution hole 1538 is occluded by the control tube 1536 as the windows 1540 are not in alignment with the elution holes 1538. In a second position in FIGS. 66C, at least one of the elution holes 1538 that were occluded in the first position is exposed as a window 1540 in the control tube 1536 is rotated to a location overlying the elution hole 1538 to allow elution of treatment agent through the elution hole 1538. Depending on the sizes and locations of the elution holes and the control tube windows, the control tube of the catheter may provide multiple positions that each allow a different elution pattern. Not every elution hole requires a corresponding window, as some holes may be open in all control tube positions. The proximal end of the control tube 1536 may have a resistance lock capable of reversibly securing the relative position of the control tube and the catheter.

In another embodiment of the invention, comprising a catheter with a slidable control tube overlying the elution holes of the catheter and is slidable in a direction along the longitudinal axis of the catheter. The control tube has an extended position whereby the control tube is positioned over the elution holes to protect the elution holes from clogging and other damage, and a withdrawn position that provides for elution of medicament out of the elution holes. The control tube is also capable of intermediate positioning between the the extended and withdrawn positions. Intermediate positioning between the extended and withdrawn positions may be configured for smooth sliding or segmented sliding. With segmented sliding, slight resistance to movement is created along regular or desired intermediate positions to provide predictable positioning of the control tube. The resistance may be created by spaced protrusions and indentations between the control tube and catheter that are capable of forming a friction fit. The proximal end of the control tube may have a resistance lock capable of reversibly securing the relative position of the control tube and the catheter.

In one embodiment of the invention, the catheter system further comprises a sterilizing filter in the flow path between the medicament source and the elution holes that is capable of filtering particles size as small as about 0.2 microns. A sterilizing filter may be particularly advantageous when the medicament comprises a foam. Techniques for producing foam-based medicaments often require the user to generate the foam at the time of the procedure by mixing the medicament with ambient air, which may contain particulates and biologically active materials. A sterilizing filter may be an integrally formed part of the catheter, or it may be attachable to the catheter, which is then attached to the medicament source for infusion into the catheter.

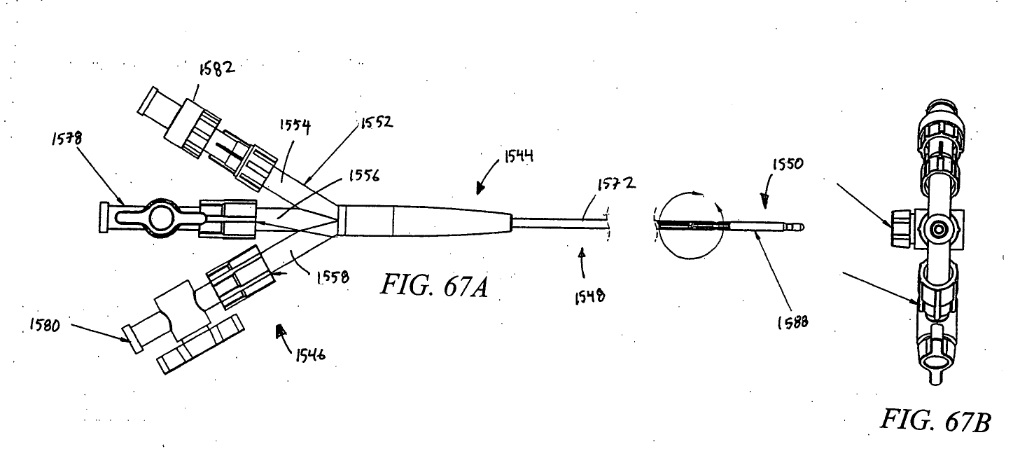

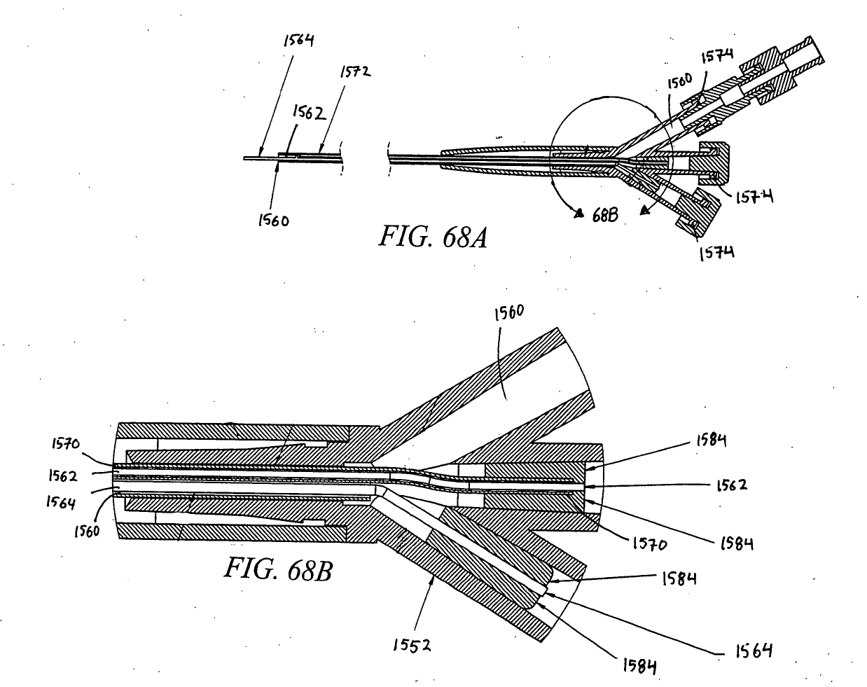

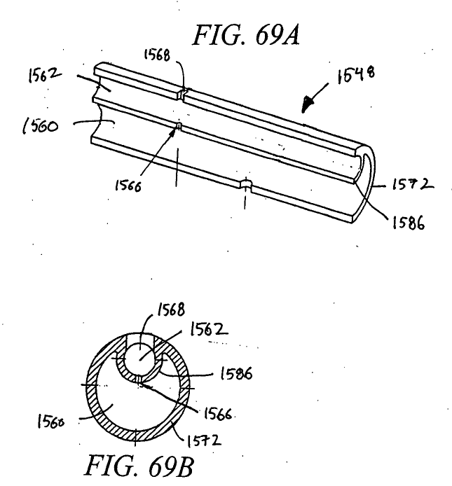

FIGS. 67A and 67B depict a preferred embodiment of the invention, with an infusion catheter 1544 comprising a proximal end 1546, a catheter body 1548 and a distal end 1550. The proximal end 1546 of the catheter 1544 comprises a trifurcated fitting 1552 with three access ports 1554, 1556, 1558, each port providing access to a lumen in the body 1548 of the catheter 1544. As shown in FIGS. 68A and 68B, the fitting 1552 and body 1548 of the catheter comprises an infusion lumen 1560, a side lumen 1562 and an inflation lumen 1564. As shown in FIGS. 69A and 69B, the catheter body 1548 comprises at least one inner elution hole 1566 and outer elution hole 1568 that allow fluid from the infusion lumen 1560 to exit the catheter. The side lumen 1562 is integral with the outer catheter wall 1572 and is positioned between at least some of the inner and outer elution holes. FIGS. 68B depicts the side lumen 1562 containing a bladder tube 1570 that is capable of blocking flow through the elution holes 1566, 1568 when the bladder tube 1570 is in an inflated state. The proximal end of the access ports 1554, 1556, 1558 may comprise a mechanical coupling 1574 for attaching other medical devices to the infusion catheter. Such devices include but are not limited to syringes, needles, stopcocks, mechanical actuators, pressure sensors, fluid samplers, intravascular ultrasound devices and other devices known in the art. In one example, shown in FIGS. 67A and 67B, a high pressure stopcock 1578 is attached to the access port 1556 contiguous with the bladder tube and a low pressure stopcock 1580 is attached to the access port contiguous with the inflation lumen. A high-pressure stopcock typically used in vascular interventions is capable of operating at up to 1000 psi; low-pressure stopcocks are typically rated at 200 psi or less. In some embodiments of the invention, the devices described above may be integrally formed with the proximal end of the catheter in any of a variety of combinations. The mechanical coupling may comprise any of a variety of mechanical couplings known in the art, including but not limited to Luer adapters. The components comprising the proximal end of the catheter may be joined or engaged using a UV-cure adhesive or sealant as is known in the art. In one embodiment, a stopcock is integrally formed in the catheter between the access port and the lumen of the catheter body to restrict fluid movement in and/or out of a catheter lumen through the access port. As shown in FIGS. 67A and 67B, a proximal end of an access port may further comprise a hemostasis valve or fluid seal 1582 for preventing leakage of bodily fluids out of the access port

FIGS. 68A and 68B depict one preferred embodiment of the invention (but without any attached stopcocks). Proximally, the bladder tube 1570 and balloon inflation lumen 1564 are surrounded by lumen seals 1584 that resist retrograde leakage of fluid from the infusion lumen 1560 around the bladder tube 1570 and inflation tube 1564. The bladder tube courses distally and enters the side lumen of the catheter body.

FIGS. 69A and 69B depict a portion of the catheter body 1548 comprising a side lumen 1562 for housing the bladder tube (not shown), the infusion lumen 1560 and the elution holes 1566, 1568. The inner hole 1566 lies within an inner wall 1586 of the catheter and the outer hole 1568 that lies in the outer wall 1572 of the catheter, adjacent to the side lumen 1562. The elution holes 1566, 1568 are capable of being blocked by a bladder tube located in the side lumen 1562. In the preferred embodiment, the inner hole 1566 has a circular cross section and a diameter of about 0.0020″. Each inner hole 1566 is aligned with an outer hole 1568, each outer hole 1568 having a length of about 0.0070″ as measured along the longitudinal length of the catheter 1544, and a width of about 0.0220″. Each pair of holes 1566, 1568 is spaced about 2 cm apart along the length of the catheter 1544. In one embodiment, the most proximal pair of holes is located about 32 cm distal from where the distal end of the trifurcated fitting is engaged to the proximal end of the catheter body. The catheter body generally comprises from about ten to about twenty-two pairs of elution hole, depending on the length of the catheter.

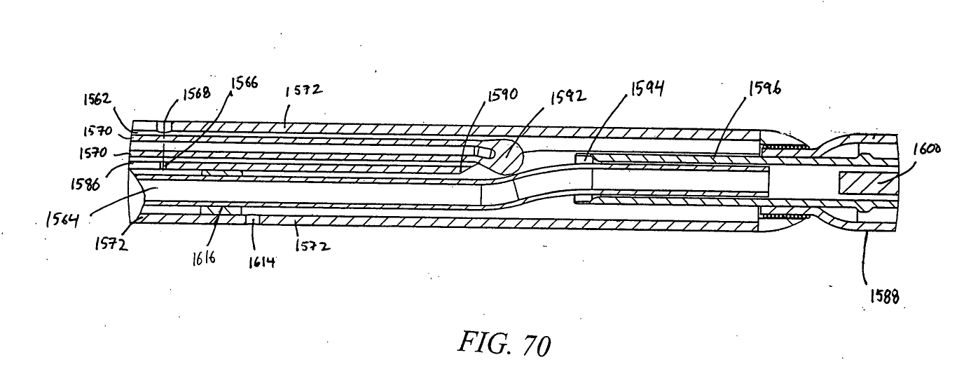

FIG. 70 depicts a preferred embodiment of the distal end of the catheter body 1572 and its attachment to the proximal end of the inflatable balloon blood vessel occluder 1588. The inflatable tube 1570 terminates just distal to the end 1590 of the side lumen 1562, the distal end of the tube 1570 comprising an enlarged bulb 1592 that seals off the end 1590 of the side lumen from the rest the distal end of the catheter body. In other embodiments of the invention, a sealant, adhesive or melting process known in the art is used to seal off the end of the inflatable tube 1570 and side lumen 1562. The balloon inflation lumen inserts into a conduit 1594 of a coupling joint 1596 that attaches the inflatable balloon 1588 to the distal end of the catheter body.

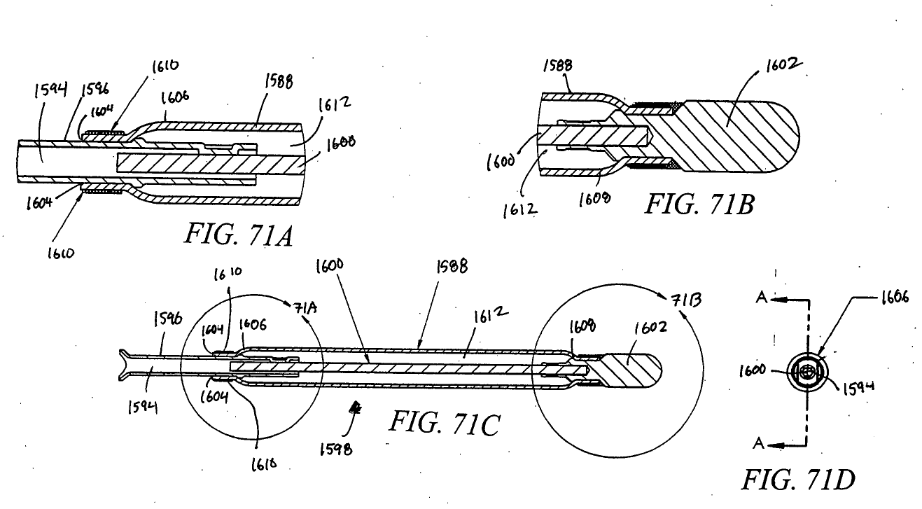

FIGS. 71A to 71D depict a preferred embodiment of the balloon assembly 1598 attached to the distal end of the catheter body. The balloon assembly 1598 comprises a proximal coupler 1596 or sleeve, a balloon support 1600, a tubular balloon material 1588 and a distal tip 1602. The coupler 1596 engages the inflation tube 1570 from the catheter body 1548 and provides a bonding surface 1604 to circumferentially bond the tubular balloon material 1588 between the coupler bonding surface 1604 and the distal end of the catheter body lumen. In one embodiment, the proximal 1606 and distal ends 1608 of the tubular balloon material 1588 are further reinforced by silk thread 1610 or a ferrule. A hermetic seal is provided between the catheter body, tubular balloon material 1588 and coupler 1594 using a sealant or adhesive known in the art, preferably a UV-bondable compound. A hermetic seal is also provided with the balloon inflation tube 1584 such that increased pressure in the inflation tube 1584 is transmittable to the inflation space 1612 within the tubular balloon material 1588. Distally, the coupler 1594 engages the balloon support 1600, which provides a stiffened core for anchoring the balloon 1588, and provides for symmetrical inflation of the balloon 1588 and to resist buckling and folding of the balloon 1588 as it is introduced into a body lumen or a introducer. In the preferred embodiment, the stiffened core 1600 comprises a cut wire, where the proximal end of the wire is engaged to the sleeve by crimping. The distal end of the wire 1600 is crimped to the proximal end of the catheter tip 1602. The tip 1602 comprises an elongate member that provides a blunt, atraumatic tip to the infusion catheter that minimizes vessel trauma as the infusion catheter is inserted into the body. The elongate member is also used to seal the distal end of the tubular balloon material 1588 to form the inflation space of the balloon assembly. In one embodiment, distal tip 1602 comprises an LED, illuminated fiber-optic line, radio-opaque material, magnetized material or other positioning identification markers to provide the in-situ localization of the distal tip during the procedure by methods previously described.

In one embodiment of the invention, a method for using a longitudinal infusion catheter is provided. The patient is placed on a flat surface and prepped and draped in the usual sterile fashion. The venous anatomy is evaluated and the insertion site is marked and selected. Tributary sites and other sites that may require additional therapy are identified and the distance measured relative to the insertion site or other similar site. Catheter integrity and function is verified by checking balloon inflation and infusion of saline, heparinized saline or other sterile fluid into the infusion lumen of the catheter. In one embodiment, the balloon is pressurized to at least about 100 psi with a syringe to purge the gaseous fluid in the distal balloon. Functionality of the elution hole controller, if provided, is checked. Local or general anesthesia is achieved as needed. Local anesthesia may be achieved with the injection of 1% lidocaine at the insertion site using a syringe with a 20 gauge to 25 gauge needle. An 18 gauge needle on a 5 mL syringe is then inserted into the anesthetized skin while aspirating. When venous blood return is confirmed, the needle is held in place as the syringe is removed. In one embodiment, a “J” wire is inserted through the needle. Resistance is checked during the wire insertion. If resistance is encountered, the needle is repositioned and wire insertion is repeated. If no resistance is encountered, wire position is maintained as the needle is removed over the wire. A vessel dilator and catheter introducer sheath is passed over the wire and optionally secured to the skin or the limb by a strap, suture or other anchoring mechanism known in the art. The wire and vessel dilator are removed from the catheter introducer sheath and replaced with the infusion catheter. In one embodiment, a catheter lock on the introducer secures the position of the catheter relative to the introducer. The limb to be treated may be raised to facilitate drainage of blood out of the vein. The position of the catheter distal tip is verified and the distal balloon is inflated, or alternatively, the distal vein occluder is activated. A 5 mL syringe with isotonic saline is attached to the balloon inflation lumen of the catheter and the plunger is fully depressed. Balloon inflation and/or blood flow across the balloon is evaluated by radiographic or other means. In one embodiment, a bolus of heparin is injected into the catheter through the infusion lumen access port while the elution holes are open to verify and maintain patency of the elution holes. In one embodiment, radio-contrast agent is injected into the blood vessel under radiographic visualization to confirm the vessel anatomy. Radio-opaque interval markers may be positioned about the leg to facilitate localization of any areas of interest visualized by the radio-contrast agent.

The sclerosing agent is prepared as needed and a 20 mL syringe filled with the agent is attached to the infusion lumen access port. A pressure dressing may be applied to the treatment area to enhance vessel wall contact during the infusion of treatment agent. In one embodiment, the infusion catheter is configured for a first elution pattern or location and an amount of agent is dispensed from the syringe and into the vessel. The treated limb may be optionally lowered to a horizontal position to facilitate even distribution of the agent during injection. The position of the limb may also be altered with respect to the level of the heart to facilitate movement of the injected migration to areas requiring enhanced sclerosing effect. In instances where a foam-based sclerosing agent is used, the treated limb may be placed in initially in an elevated position to enhance drainage of venous blood from the limb, then placed below the heart during injection to facilitate migration of the foam-based sclerosant to the saphenofemoral junction to provide increased sclerosing effect. In one embodiment, the catheter is reconfigured for another elution pattern or location and additional agent is injected into the vessel. The reconfiguration of the catheter and dispensing of agent is repeated as needed. In one embodiment, treatment effect is evaluated between injections and additional treatment sites may be identified. The catheter is reconfigured to elute agent at the additional sites and additional treatment agent is injected. In one embodiment, heparin boluses or other anti-coagulation agent are infused through the infusion lumen and elution holes of the catheter between injections of the sclerosing agent or radio-contrast agent to maintain patency of the infusion catheter. The distal balloon of the catheter is deflated and the catheter is withdrawn from the patient. The introducer is removed from the insertion site and hemostasis is achieved by placing one or more non-absorbable sutures to close the insertion site. The insertion site is cleaned with alcohol and dressed. A pressure dressing or wrap is applied around treated limb as needed.

In one embodiment of the invention, a method for using an infusion catheter with an occludable bladder tube is provided. The patient is placed on a flat surface and prepped and draped in the usual sterile fashion. The venous anatomy is evaluated and the insertion site is marked and selected. Tributary sites and other sites that may require additional therapy are identified and the distance measured relative to the insertion site or other similar site. Catheter integrity and function is verified by checking balloon inflation and infusion of saline, heparinized saline or other sterile fluid into the infusion lumen of the catheter. In one embodiment, the balloon is pressurized to at least about 100 psi with a syringe to purge the gaseous fluid in the distal balloon. Integrity of the bladder tube is assessed by inflating the bladder tube and verifying occlusion of the elution holes by the bladder tube. The bladder tube is deflated and reopening of the elution holes is rechecked. Local or general anesthesia is achieved as needed. Local anesthesia may be achieved with the injection of 1% lidocaine at the insertion site using a syringe with a 20 gauge to 25 gauge needle. An 18 gauge needle on a 5 mL syringe is then inserted into the anesthetized skin while aspirating. When venous blood return is confirmed, the needle is held in place as the syringe is removed. In one embodiment, a “J” wire is inserted through the needle. Resistance is checked during the wire insertion. If resistance is encountered, the needle is repositioned and wire insertion is repeated. If no resistance is encountered, wire position is maintained as the needle is removed over the wire. A vessel dilator and catheter introducer sheath is passed over the wire and optionally secured to the skin or the limb by a strap, suture or other anchoring mechanism known in the art. The bladder tube is reinflated to occlude the elution holes. The wire and vessel dilator are removed from the catheter introducer sheath and replaced with the infusion catheter. In one embodiment, a catheter lock on the introducer secures the position of the catheter relative to the introducer. The position of the catheter distal tip is verified and the distal balloon is inflated. A 5 mL syringe with isotonic saline is attached to the balloon inflation lumen of the catheter and the plunger is fully depressed. Balloon inflation and/or blood flow across the balloon is evaluated by radiographic or other means. In one embodiment, a bolus of heparin is injected into the catheter through the infusion lumen access port while the elution holes are open to verify and maintain patency of the elution holes. In one embodiment, radio-contrast agent is injected into the blood vessel under radiographic visualization to confirm the vessel anatomy. The bladder tube is deflated prior to injection of heparin and/or radio-contrast agent and reinflated after injection. Radio-opaque interval markers may be positioned about the leg to facilitate localization of any areas of interest visualized by the radio-contrast agent. In another embodiment, Doppler ultrasound is used to confirm vessel occlusion. In one embodiment, use of Doppler ultrasound is preferred because it reduces the need to deflate and reinflate the bladder tube. Reductions in the use of the bladder tube during the procedure may decrease the exposure of the elution holes to the vessel and decrease the risk of occlusion.

The sclerosing agent is prepared as needed and a 20 mL syringe filled with the agent is attached to the infusion lumen access port. In one embodiment, a pressure dressing is applied to the treatment area to enhance vessel wall contact during the infusion of treatment agent. The bladder tube is deflated and an amount of agent is dispensed from the syringe and into the vessel. The bladder tube is reinflated. In one embodiment, the operator reconfigures and/or repositions the catheter for another elution pattern or location, deflates the bladder tube, injects additional agent into the vessel, and reinflates the bladder tube. The cycle is repeated as needed to achieve the desired treatment parameters. In one embodiment, treatment effect is evaluated between injections and additional treatment sites may be identified. In one embodiment, heparin boluses or other anti-coagulation agent are infused through the infusion lumen and elution holes of the catheter after injections of the sclerosing agent or radio-contrast agent to maintain patency of the infusion catheter. The distal balloon of the catheter is deflated and the catheter is withdrawn from the patient. The introducer is removed from the insertion site and hemostasis is achieved by placing one or more non-absorbable sutures to close the insertion site. The insertion site is cleaned with alcohol and dressed. A pressure dressing or wrap is applied around treated limb as needed.

In one embodiment of the invention a kit or system for performing sclerotherapy is provided. In one embodiment, the kit comprises an infusion catheter with an elution zone along at least a 15 cm longitudinal length of the catheter, an infusion syringe and a distal balloon inflation syringe. In another embodiment, the kit comprises an infusion catheter with a plurality of longitudinally arranged elution lumena, 5 ml solution of 1% lidocaine with 1:100,000 epinephrine, an 18-gauge needle and 5 mL syringe, a J-wire, a catheter sheath introducer, a vessel dilator, a treatment agent foaming device, a foam sterilizing filter, a bladder tube syringe, a balloon inflation syringe and a treatment agent infusion syringe. In another embodiment of the invention, the kit or system comprises an infusion catheter capable of accepting a movable wire occluder and a plurality of insertable wire occluders of different configurations.

In one embodiment of the invention, the catheter with a side lumen may be fabricated as a single, integral structure, with the side lumen comprising a longitudinal hole within the sidewall of the catheter. Such a catheter may be manufactured as a dual-lumen catheter by processes including but not limited to extrusion with a dual-air mandrel extrusion tip and die, or extrusion with an air-mandrel tip for the main catheter lumen and a removable wire mandrel for the smaller side lumen. If a wire mandrel, typically made from copper or silver-plated copper, is used to form a lumen, the wire is typically removed from cut lengths of catheter tubing by stretching and breaking the wire to remove the wire from the lumen. One skilled in the art will understand that other such techniques may be used to form catheter tubing with one or more lumena.

The catheter tubing may be made from PTFE, FEP, PFA, Pebax®, polyurethane, nylon, PVC, TPE, polyester and any of a variety of other polymers known in the art. In one embodiment, a catheter material with hydrophobic properties may be preferred, because such materials tend to stabilize foam medicaments better than hydrophilic materials. A single material may be used to form the catheter tubing, or more than one material may be used. In another embodiment, multiple materials are used to form the catheter tubing. In one embodiment, the inner wall material is different from the outer wall material of the infusion catheter. In one embodiment, a tube of a second material may be disposed within the wall of the catheter. In one example, the side lumen of the catheter is first formed by extrusions, then the remaining portions of the catheter are then extrudes with the pre-formed side lumen. In one embodiment, the pre-formed side lumen preferably comprises a material that has a higher melting temperature than the material from which the other portion of the catheter tube is extruded, to reduce melting and/or distortion of the side lumen during the catheter tube extrusion. In one example of a dual-lumen catheter tube, a tubing of FEP or PTFE with an inside diameter of 0.025″ and an outside diameter of 0.031″ is used for the side lumen, which can be incorporated into the wall of an extruded catheter tubing of polyurethane.

In one embodiment of the invention, the elution holes may be formed through thermal punching, wherein a heated wire punch of the desired diameter is pushed through the sidewall of the catheter and withdrawn, leaving a hole. In one embodiment, the temperature of the wire punch is controlled so that when the catheter material is displaced, but adjacent regions of the catheter do not undergo significant melting. In one preferred embodiment, the wire punch is tapered to add stiffness and strength to the wire punch while having the capability of forming smaller holes. For example, a wire may be tapered from 0.008″ to 0.001″ and pushed through the sidewall of the catheter so that the wire penetrates slightly beyond the inner surface of the catheter, resulting in a hole of about 0.002″ at the smallest point. The wire punch can have any of a variety of cross-sectional shapes, including but not limited to circles, ovals, squares, rectangles, other polygons, or a combination thereof.

In one embodiment of the invention, a laser is used to drill from the exterior surface of the catheter, through the side lumen and to the infusion lumen to form the inner holes and outer holes. Small holes, of about 8 microns or less, may be drilled with lasers. Pulse lasers capable of delivering very high power levels for very short periods are preferably used, but such lasers are not required. High power levels and short pulse durations result in ablation, evaporation, and/or photodissociation of the catheter materials rather than melting. Such pulses can be provided with Q-switched YAG lasers at natural frequencies or a multiple thereof, or by excimer lasers, such as xenon fluoride lasers. With high-powered laser drilling, hole size may be controlled by using near-field focusing, beam apertures, and/or focal-length control. In one embodiment, holes may be of substantially constant diameter or may vary in diameter through the wall of the catheter. Larger holes may be formed by defocusing the beam, near-field focusing a larger aperture, and/or by moving either the catheter or the laser beam to remove material and form a larger hole.

In one embodiment, where infusion catheters comprise inner holes and outer holes, the inner and outer holes may be made with different sizes and different methods. In one embodiment, the outer holes may also be formed by catheter manufacturing techniques such as traditional punching, grinding or drilling. The wall thickness of the catheter in the selected location of the hole may also be reduced by skiving, where a portion of the catheter wall thickness is sliced off.

In one embodiment, if the infusion catheter is configured with inner holes that are generally aligned with the outer holes, the inner holes and outer holes may be drilled or punched at the same time as the outer holes.

In one embodiment, wherein the infusion catheter is configured so that the inner holes are not aligned with the outer holes, the inner holes can be formed by laser drilling or thermal punching through the outer catheter wall. The hole through the outer catheter wall may be closed off by thermal sealing or by the use of a sealant, such as a solvent, solvent cement, UV-cure adhesive, epoxy or any of a variety of adhesive materials. In one embodiment, non-aligned inner holes and outer holes may be formed by extruding the catheter tube over a preformed side lumen tube having pre-drilled or pre-punched inner hole lumena.

In one embodiment of the invention, the catheter is constructed with the use of rigid ferrules of metal or hard plastic at the distal end and proximal end of the inflatable occlusion balloon. To maintain a catheter of a small size with the desired flexibility and stiffness to be introduced to the desired location in the body, the catheter body tubing preferably has thickness of about 0.010″ or more to resist collapsing from the pressure of the fiber winding. In other embodiments of the invention, the catheter body tubing has a wall thickness of about 0.004″ to about 0.012″. In one embodiment, thin metal tubing, such as stainless steel extra-thin-wall hypodermic tubing, may be used as a ferrule onto which the balloon is tied and bonded. In one embodiment, silk thread or a plastic ferrule is used to bond the balloon. These ferrules may be bonded to the inflation tubing and sealed within the catheter outer tubing by a sealant, including but not limited to an acrylic adhesive or UV-curable urethane. Such a construction is preferable because it is conducive to good manufacturing practice (“GMP”), as it allows the balloon-ferrule subassembly to be fabricated separately and tested prior to incorporation into the catheter assembly.

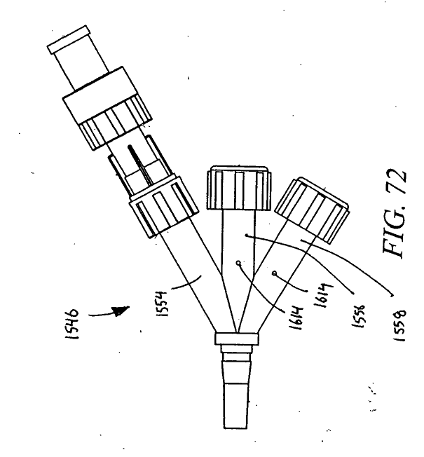

To bond the parts of the infusion catheter during the manufacturing process, any of a variety of sealants and adhesives may be used, in addition to welding or other techniques known in the art. In the preferred embodiment of the invention, a UV-cure adhesive is used to bond the subparts of the catheter. To access inner areas of the catheter for bonding, access holes may be provided in the catheter. FIGS. 69A and 70 depict embodiments of the invention with access conduits 1614 for injecting adhesive into the catheter. FIG. 72 shows access conduits 1614 placed in the access ports 1556, 1558 of the trifurcated fitting 1546 in FIGS. 68A and 68B. The access conduits 1614 allow insertion of the adhesive or sealant around the bladder tube and balloon inflation tube and prevent retrograde leakage of the infusion lumen contents from out of these access ports. After sealing is complete, these access conduits may be closed by thermal sealing or by the use of a sealant, such as a solvent, solvent cement, UV-cure adhesive, epoxy or any of a variety of adhesive materials.

To limit the flow of adhesive or sealant into unintended portions of the catheter during the manufacturing process, dams may be used in the catheter design to aid the manufacturing process without reducing the functionality of the catheter. In one example in FIG. 70, a distal dam 1616 surrounds the balloon inflation tube 1564 distal to the most distal elution hole 1566. The distal dam 1616 resists any retrograde flow of adhesive or sealant used to seal the balloon assembly that may affect the function of the catheter. The distal end of the side lumen terminates distal to the distal dam.

There have been described and illustrated herein several embodiments of methods and apparatus for treating the interior of a blood vessel. While particular embodiments of the invention have been described, it is not intended that the invention be limited thereto, as it is intended that the invention be as broad in scope as the art will allow and that the specification be read likewise. Thus, it will be appreciated that the methods and apparatus of the invention may be used in different combinations. It will therefore be appreciated by those skilled in the art that yet other modifications could be made to the provided invention without deviating from its spirit and scope as so claimed. For all of the embodiments described above, the steps of the methods need not be performed sequentially.

1. A device for treating blood vessels, comprising:
an elongate body having a proximal end, a distal end, and an infusion lumen extending there through;
a plurality of elution holes, in valved communication with the infusion lumen; and
a wall which is movable between a first position in which the wall blocks communication between the infusion lumen and the elution holes, and a second position in which the infusion lumen is in communication with the elution holes.

2. The device of claim 1, wherein the wall is movable in response to a change in pressure.

3. The device of claim 1, wherein the wall is movable in response to introduction of an inflation media.

4. The device of claim 1, wherein the wall is in the form of an inflatable tube.

5. The device of claim 4, further comprising a side lumen on the body, and wherein the inflatable tube is positioned within the side lumen.

6. The device of claim 4, wherein the tube is positioned within the infusion lumen.

7. The device of claim 4, wherein the inflatable tube has an axial length of at least about 0.5 cm.

8. The device of claim 1, wherein the total fluid resistance of the elution holes is about equal to or greater than the total fluid resistance of the infusion lumen.

9. The device of claim 1, wherein the total fluid resistance of the elution holes is at least about 125% of the fluid resistance of the infusion lumen.

10. The device of claim 1, wherein the average hydraulic diameter of the elution holes is less than about 0.010″.

11. The device of claim 1, wherein the average hydraulic diameter of the elution holes is less than about 0.004″.

12. The device of claim 1, wherein the average spacing between elution holes is within the range of from about 1 cm to about 2 cm.

13. The device of claim 1, further comprising an inflatable occlusion balloon carried by the distal end of the body.

14. The device of claim 1, further comprising a guidewire lumen extending axially through at least a portion of the length of the elongate body.

15. The device of claim 5, wherein the inflatable tube has a deflated diameter, the side lumen has an inside diameter, and the deflated diameter is no more than about 75% of the inside diameter.

16. A fluid delivery catheter, comprising:
an elongate, flexible tubular body, having a proximal end and a distal end;
an infusion lumen extending through the body from the proximal end in the direction of the distal end;
at least two infusion ports on the tubular body; and
an inflatable tube within the tubular body;
wherein at least one infusion port is in communication with the infusion lumen when the inflatable tube is in a first inflation state, and the infusion port is isolated from the infusion lumen when the inflatable tube is in a second inflation state.

17. A fluid delivery catheter as in claim 16, further comprising a vascular occlusion balloon on the distal end of the tubular body.

18. A fluid delivery catheter as in claim 17, further comprising a proximal manifold having an infusion port in communication with the infusion lumen, and an inflation port in communication with the occlusion balloon.

19. A method of treating a body lumen, comprising:
providing a catheter with an infusion lumen and a plurality of elution holes in selective communication with the infusion lumen, the catheter having a first configuration adapted to resist flow through at least one elution hole and a second configuration adapted to allow flow through the at least one elution hole;
inserting the catheter into a patient;
introducing a therapeutic fluid into the infusion lumen; and
changing the catheter from the first configuration to the second configuration to permit escape of therapeutic fluid through the at least one elution hole.

20. A method of treating a body lumen as in claim 19, wherein the changing the catheter step comprising moving a movable wall from a first position in which communication between the at least one elution hole and the infusion lumen is interrupted, to a second position in which the at least one elution hole is in communication with the infusion lumen.

21. A method of treating a body lumen as in claim 20, wherein the changing the catheter step comprises deflating a tubular flow regulator.

22. A method of introducing a therapeutic agent into a vein, comprising the steps of:
introducing a catheter into the vein, the catheter having a plurality of infusion ports and an infusion lumen;
activating an occlusion device on the catheter to occlude blood flow within the vein;
removing a barrier from at least one of the plurality of infusion ports; and
infusing therapeutic agent from the infusion lumen, through the ports and into the vein.

23. A method of introducing a therapeutic agent into a vein as in claim 22, wherein the introducing step comprises introducing the catheter into the saphenous vein.

24. A method of introducing a therapeutic agent into a vein as in claim 23, wherein the introducing step comprises introducing the catheter into the saphenous vein in the vicinity of the knee.

25. A method of introducing a therapeutic agent into a vein as in claim 23, wherein the introducing step comprises introducing the catheter into the saphenous vein in the vicinity of the ankle.

26. A method of introducing a therapeutic agent into a vein as in claim 22, wherein the activating an occlusion device step comprises inflating an occlusion balloon.

27. A method of introducing a therapeutic agent into a vein as in claim 22, wherein the activating an occlusion device step is accomplished to isolate the saphenofemoral junction from the infusion ports.

28. A method of introducing a therapeutic agent into a vein as in claim 22, wherein the removing a barrier step comprises deflating an elongate, tubular bladder.

29. A method of introducing a therapeutic agent into a vein as in claim 22, further comprising enhancing drainage of the vein by raising the position of the vein relative to the location of the occlusion device.

30. A method of introducing a therapeutic agent into a vein as in claim 22, further comprising lowering the position of the vein relative to the location of the occlusion device to facilitate migration of therapeutic agent along the vein; wherein the therapeutic agent is a foam.

31. A method of introducing a therapeutic agent into a vein as in claim 22, further comprising maintaining a raised position of the vein relative to the location of the occlusion device to facilitate migration of the therapeutic agent to the saphenofemoral junction.

32. A method of inhibiting retrograde flow of body fluid through the effluent ports and into the infusion lumen of a catheter, comprising the steps of:
providing a fluid delivery catheter, having an elongate body, at least one effluent port on the body and an infusion lumen extending within the body;
inflating a flow regulator within the tubular body to isolate the effluent port from the infusion lumen; and
introducing the catheter into a patient in a location that exposes the catheter to a body fluid;
wherein the flow regulator inhibits retrograde flow of body fluid through the effluent port and into the infusion lumen.

33. A method of inhibiting retrograde flow of body fluid as in claim 32, wherein the inflating a flow regulator step comprises inflating an elongate tubular balloon.

34. A method of inhibiting retrograde flow of body fluid as in claim 32, additionally comprising the step of deflating the flow regulator to place the effluent port in communication with the infusion lumen.
